(19) United States
(12) Patent Application Publication
(10) Pub. No.: US 2021/0205225 A1
(43) Pub. Date: Jul. 8, 2021

(54) BIOAVAILABLE ORAL DOSAGE FORMS

(71) Applicant: PTC Therapeutics, Inc., South Plainfield, NJ (US)

(72) Inventors: Mandar V. DALI, Bridgewater, NJ (US); Akm Nasir UDDIN, Somerset, NJ (US)

(21) Appl. No.: 17/264,940

(22) PCT Filed: Aug. 2, 2019
(86) PCT No.: PCT/US2019/044853

§ 371 Date: Feb. 1, 2021

Related U.S. Application Data
(60) Provisional application No. 62/714,182, filed on Aug. 3, 2018.

(51) Int. Cl. A61K 9/20; A61K 9/48; A61K 31/437; A61K 9/00

(52) U.S. Cl. CPC A61K 9/2027; A61K 9/4866; A61K 31/437; A61K 9/4883; A61K 9/0053; A61K 9/2095; A61K 9/2054

(57) ABSTRACT

The invention relates to bioavailable pharmaceutical compositions having increased dose loading and improved dissolution less subject to a food effect.

### FIELD OF THE INVENTION

A form of a lipophilic Compound useful in a pharmaceutical composition and a method of forming a solid dispersion, such as a spray dried intermediate, with the form of the Compound are described. Also described is the use of the solid dispersion to provide a bioavailable oral dosage form having increased dose loading and improved dissolution less subject to a food effect.

### BACKGROUND OF THE INVENTION

The bioavailability of an orally administered therapeutic agent is the degree to which the agent is absorbed in the human body and becomes available to an in vivo target (e.g., for interaction or complexation and the like) at a target site (e.g., in or on a cell and the like). To be made bioavailable, a therapeutic agent generally needs to have a certain aqueous solubility with respect to the dose being administered, thus, it would be desirable for the agent to be more soluble in water (hydrophilic) than in fat (lipophilic). Generally, lipophilic agents are poorly soluble in water. Therefore, amongst other factors, the degree of an agent's lipophilicity determines the agent's bioavailability.

As a result, there remains a continuing need in the art and a continuing demand in the market for pharmaceutical compositions having ease of dosing, increased dose loading and improved dissolution and bioavailability that are useful for a particular agent.

### SUMMARY OF THE INVENTION

Encompassed herein is a form of a lipophilic Compound having Formula (I) set forth herein, known as 4-chlorophenyl (S)-6-chloro-1-(4-methoxyphenyl)-1,3,4,9-tetrahydro-2H-pyrido[3,4-b]indole-2-carboxylate (“Compound 1”).

In one aspect, the form of Compound 1 is in an amorphous form.

In another aspect, the form of Compound 1 is a crystalline form.

The use of the form of Compound 1 in preparing a solid dispersion, such as a spray dried intermediate, comprising an amorphous form of Compound 1 and a polymer is described, wherein the polymer used is a hydrophilic polymer.

In one aspect, the polymer used is polyvinyl pyrrolidone (PVP) or hydroxypropyl methyl cellulose (HPMC).

In one aspect, the form of Compound 1 used in preparing the spray dried intermediate is an amorphous form. In another aspect, the form of Compound 1 used in preparing the intermediate is a crystalline form.

Also encompassed is a method for preparing a solid dispersion, such as a spray dried intermediate comprising an amorphous form of Compound 1 and a polymer.

In one aspect, the method includes co-dissolving Compound 1 and the polymer in a solvent system to form a liquid dispersion with subsequent solvent removal.

In one aspect, the intermediate formed is a solid dispersion.

In one aspect, the solvent is removed by spray drying. In another aspect, the amorphous form of Compound 1 is formed as the spray dried intermediate is obtained.

The use of the spray dried intermediate in a pharmaceutical composition comprising the spray dried intermediate in intimate admixture with one or more pharmaceutically acceptable excipients to provide a bioavailable oral dosage form is also described.

In another aspect, the intermediate is a spray dried intermediate comprising an amorphous form of Compound 1 and a hydrophylic polymer. In another aspect, hydrophylic polymer is PVP or HPMC. In another aspect, the PVP is polyvinylpyrrolidone K-30 (PVP K-30). In another aspect, the HPMC is HPMC E5.

In one aspect, the dosage form is an oral solid dosage form. In another aspect, the oral dosage form is a tablet. In another aspect, the oral dosage form is a capsule.

The use of the bioavailable oral dosage form in a weight based dosing regimen, wherein the dosing regimen maintains a target plasma concentration, is also described.

The use of the bioavailable oral dosage form in a fixed dose regimen, wherein the regimen maintains a target plasma concentration, is also described.

Administration of the oral dosage form with food to enhance bioavailability is also described.

Accordingly, the present application provides pharmaceutical compositions having increased dose loading and improved solubility.

### BRIEF DESCRIPTION OF THE DRAWINGS

FIG. 1 shows the dissolution rates of encapsulated dry blend formulations of spray dried intermediates (SDI) in 0.1 N HCl containing 1.5% sodium dodecyl sulfate (SDS) as a function of time, at various levels of dose loading with various polymer and excipient combinations.

FIG. 2 shows comparative dissolution rates of encapsulated dry blend formulations of SDIs in 0.1 N HCl containing 1.5% SDS in two different volumes of dissolution fluid as a function of time, at various levels of dose loading with various polymer and excipient combinations.

FIG. 3 shows the dose normalized plasma concentration as a function of time of encapsulated dry blend formulations of SDIs tested in a preclinical in vivo oral bioavailability pharmacokinetic animal study.

FIG. 4 shows the dose normalized plasma concentrations of SDIs used in tablet and capsule formulations as a function of time in a preclinical in vivo oral bioavailability pharmacokinetic animal study.

FIG. 5 shows the dose normalized plasma concentrations of SDIs used in tablet and capsule formulations as a function of time in fed animals in a preclinical in vivo pharmacokinetic food effect animal study.

FIG. 6 shows the dose normalized plasma concentrations of SDIs used in tablet and capsule formulations as a function of time in fasted animals in a preclinical in vivo pharmacokinetic food effect study.

FIG. 7 shows the average plasma concentrations of SDIs used in tablet and capsule formulations as a function of time in fed and fasted animals in a preclinical in vivo pharmacokinetic food effect study.

FIG. 8 shows the average plasma concentrations of a Lipid Capsule Formulation as a function of time at Stage 1 of an in vivo pharmacokinetic food effect clinical study.

FIG. 9 shows the average plasma concentrations of a Lipid Capsule Formulation as a function of time in an in vivo pharmacokinetic food effect clinical study in fed and fasted subjects.

FIG. 10 shows the average plasma concentrations of a Lipid Capsule Formulation and a PVP Tablet Formulation as a function of time in an in vivo pharmacokinetic food effect clinical study.

FIG. 11 shows the average Compound 1 (“Cpd 1”) plasma concentrations obtained after administration of dose levels of 400 mg, 800 mg and 1000 mg of the PVP Tablet Formulation as a function of time in an in vivo pharmacokinetic food effect clinical study.

FIG. 12 shows the average plasma concentrations at dose levels of 400 mg and 1000 mg of the PVP Tablet Formulation as a function of time in an in vivo pharmacokinetic food effect clinical study in fed and fasted subjects.

### DETAILED DESCRIPTION OF THE INVENTION

Encompassed herein is a form of a compound (Compound I) having Formula (I):

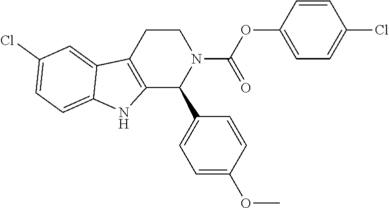 [SMILES: COc1ccc([C@H]2c3[nH]c4ccc(Cl)cc4c3CCN2C(=O)Oc2ccc(Cl)cc2)cc1]

In one aspect, Compound 1 is in an amorphous form.

In another aspect, the form of Compound 1 is a crystalline form.

The use Compound 1 in preparing a solid dispersion, such as a spray dried intermediate comprising an amorphous form of Compound 1 and a polymer is described, wherein the polymer used is a hydrophilic polymer.

In one aspect, the polymer used is PVP or HPMC. In another aspect, the PVP is PVP-K30. In another aspect, the HPMC is HPMC E5.

In one aspect, the form of Compound 1 used in preparing the intermediate is an amorphous form. In another aspect, the form of Compound 1 used in preparing the intermediate is a crystalline form.

Also encompassed is a method for preparing a solid dispersion, comprising an amorphous form of Compound 1 and the polymer.

In another aspect, the method includes co-dissolving Compound 1 and the polymer in a solvent system to form a liquid dispersion then removing the solvent.

In another aspect, the intermediate formed is a solid dispersion.

In another aspect, the solvent is removed by spray drying. In another aspect, the amorphous form of Compound 1 is formed as a spray dried intermediate is obtained.

In another aspect, the intermediate is a spray dried intermediate comprising an amorphous form of Compound 1 and a hydrophylic polymer. In another aspect, hydrophylic polymer is PVP or HPMC. In another aspect, the PVP is polyvinylpyrrolidone K-30 (PVP K-30). In another aspect, the HPMC is HPMC E5.

The use of the spray dried intermediate in a pharmaceutical composition comprising the spray dried intermediate in intimate admixture with one or more pharmaceutically acceptable excipients to provide a bioavailable oral dosage form is also described.

In another aspect, the oral dosage form is a tablet.

The use of the bioavailable oral dosage form in a weight based or fixed dose dosing regimen, wherein the dosing regimen maintains a target plasma concentration, is also described.

### Definitions

As used herein, the term “cocrystal(s)” refers to a crystal, often a large-molecule crystal, having two or more distinct molecular components within the crystal comprising a Compound provided herein and one or more suitable pharmaceutically acceptable non-toxic counterions.

As used herein, the terms “Compound 1” refers to a compound of Formula (I) described herein and pharmaceutically acceptable polymorphs or an amorphous form thereof. In certain aspects, the terms refer to a polymorph of Formula (I). In certain aspects, the terms refer to an amorphous form of Formula (I). A method of making Compound 1 is provided in International Application Publication No. WO 2005/089764.

Compound 1 provided herein is further described in U.S. Pat. No. 7,601,840 (having corresponding International Application Publication No. WO2005/089764), U.S. Pat. No. 7,767,689 (having corresponding International Application Publication No. WO2006/113703), International Application Publication No. WO2010/138758; U.S. Pat. No. 8,076,352 (having corresponding International Application Publication No. WO2008/127715); U.S. Pat. Nos. 8,076,353; 8,367,694; U.S. Publication No. 2010/0158858 (having corresponding International Application Publication No. WO2008/127714); each of which is incorporated by reference herein in its entirety. As used herein, the term “effective amount,” in the context of administering a Compound to a subject having a condition described herein, refers to the amount of a Compound that results in a beneficial or therapeutic effect. In specific aspects, an “effective amount” of a Compound refers to an amount of a Compound which is sufficient to achieve at least one, two, three, four or more of the following effects: (i) the reduction or amelioration of the severity of one or more symptoms associated with a condition described herein; (ii) the reduction in the duration of one or more symptoms associated with a condition described herein; (iii) the prevention in the recurrence of a tumor or one or more symptoms associated with a condition described herein; (iv) the regression of a condition described herein and/or one or more symptoms associated therewith; (v) the reduction in hospitalization of a subject; (vi) the reduction in hospitalization length; (vii) the increase in the survival of a subject; (viii) the inhibition of the progression of a condition described herein and/or one or more symptoms associated therewith; (ix) the enhancement or improvement of the therapeutic effect of another therapy; (x) a reduction in leukemic proliferation before surgery; (xiv) eradication, removal, or control of leukemic proliferation; (xv) a decrease in the rate of leukemic proliferation; (xvi) a reduction in mortality; (xvii) an increase in tumor-free survival rate of patients; (xviii) an increase in progression free survival; (xix) an increase in the number of patients in remission; (xx) a decrease in hospitalization rate; (xxi) the size of the tumor is maintained and does not increase or increases by less after administration of a standard therapy as measured by conventional methods available to one of skill in the art, such as magnetic resonance imaging (MRI), dynamic contrast-enhanced MRI (DCE-MRI), X-ray, computed tomography (CT) scan, a positron emission tomography scan or other imaging modalities; (xxii) the prevention of the development or onset of a condition described herein or one or more symptoms associated therewith; (xxiii) an increase in the length of remission in patients; (xxiv) the reduction in the number of one or more symptoms associated with a condition described herein; (xxv) an increase in symptom-free survival of patients having a condition described herein; (xxv.i) an increase in disease-free survival of patients having a condition described herein; (xxvi) a decrease in the concentration of circulating DHODH (dihydroorotate dehydrogenase) in the plasma, serum or other biofluids of a subject with a condition described herein; (xxvii) a decrease in circulating tumor cells (CTCs) in the blood of a subject with having a condition described herein; (xxvii.i) a decrease in circulating DNA or RNA associated with tumor cells in the blood of a subject having a condition described herein; (xxviii) a decrease in the concentration of DHODH in a biological specimen (e.g., the plasma, serum, urine, cerebrospinal fluid (CSF)) or other biofluids of a subject having a condition described herein; (xxviii) a preventing tumor vasculature following surgery; (xxix) improvement in neural function, e.g., hearing, balance, tinnitus, or vision; (xxx) inhibition or reduction in pathological production of DHODH; (xxxi) stabilization or reduction of peritumoral inflammation or edema in a subject; (xxxii) reduction of the concentration of DHODH or other angiogenic or inflammatory mediators (e.g., cytokines or interleukins) in biological specimens (e.g., plasma, serum, cerebral spinal fluid, urine, or any other biofluids); (xxxiii) inhibition or decrease in tumor metabolism or perfusion; (xxxiv) inhibition or decrease in angiogenesis or vascularization; (xxxv) improvement in quality of life as assessed by methods well known in the art (e.g., by symptom or quality of life questionnaires). In specific aspects, an “effective amount” of a Compound refers to an amount of a Compound specified below.

As used herein, the term “elderly human” refers to a human 65 years or older.

As used herein, the term “middle-aged human” refers to a human between the ages of 30 and 64.

As used herein, the term “human adult” refers to a human that is 18 years or older.

As used herein, the term “human child” refers to a human that is 1 year to 18 years old.

As used herein, the term “human toddler” refers to a human that is 1 year to 3 years old.

As used herein, the term “human infant” refers to a newborn to 1 year old year human.

As used herein, the term “hydrophilic polymer” refers to organic polymers of repeating monomers containing hydrophilic groups such as hydroxyl groups. In the context of the invention described herein, the length of the polymer and correlative viscosity is relevant, i.e., polymers with higher molecular weight tend to be more viscous. For use in preparing an intermediate comprising the form of the Compound and a polymer, the length of the useful polymer is limited by viscosity. The selected polymers are of low viscosity and may have some surfactant properties; that is, the polymer has the ability to lower either surface tension and interact with both hydrophobic and hydrophilic substances. In the present context, the ability of the polymer to have an enhanced amphiphilic character may be enhanced by the presence of one or more surfactants. Accordingly, the properties of the selected polymer, in the context of the Compound and the presence of optional excipients, are balanced to ensure that the water-insoluble, lipophilic nature of the Compound particles is overcome while aggregation and formation of fibers is avoided.

As used herein, the term “condition described herein” refers to an acute myeloid leukemia (AML), including acute myelocytic leukemia, acute myelogenous leukemia, acute granulocytic leukemia, and acute non-lymphocytic leukemia capable of being affected by DHODH inhibition. It also refers to inflammatory diseases, including, but not limited to, rheumatoid arthritis and multiple sclerosis. As used herein, the terms “subject” and “patient” are used interchangeably to refer to an individual being treated for a condition described herein. In a specific aspect, the individual is a human.

As used herein, the terms “therapies” and “therapy” can refer to any protocol(s), method(s), compositions, formulations, and/or agent(s) that can be used in the prevention, treatment, management, or amelioration of a condition or disorder or symptom thereof (e.g., a condition described herein; a condition or a symptom or condition associated therewith) described herein. In certain aspects, the terms “therapies” and “therapy” refer to biological therapy, supportive therapy, and/or other therapies useful in treatment, management, prevention, or amelioration of a condition or disorder or a symptom thereof described herein (e.g., a a symptom or condition described herein associated therewith; a condition or a symptom or condition described herein associated therewith). In certain aspects, the term “therapy” refers to a therapy other than a Compound or pharmaceutical composition thereof. In specific aspects, an “additional therapy” and “additional therapies” refer to a therapy other than a treatment using a Compound or pharmaceutical composition.

As used herein, the terms “pathologic,” “pathological” or “pathologically-induced,” in the context of the production of DHODH described herein, refer to the oncongenic transformation-induced expression of DHODH by tumor cells or other cells in the tumor environment is encompassed by the terms. In another aspect, expression of DHODH in a chronic or traumatic inflammatory condition is encompassed by the terms. In another aspect, in response to environmental stimuli, cells that disregulate or overproduce DHODH is also encompassed by the terms. As applicable, expression of DHODH supports inflammation, angiogenesis and tumor growth. The inhibition or reduction in pathological production of DHODH by a Compound can be assessed in cell culture and/or animal models, tumor tissue homogenates, blood samples, urine samples, CSF and the like, as described herein.

As used herein, the term “about” means a range around a given value wherein the resulting value is substantially the same as the expressly recited value. In one aspect, “about” means within 25% of a given value or range. For example, the phrase “about 70% by weight” comprises at least all values from 52% to 88% by weight. In another aspect, the term “about” means within 10% of a given value or range. For example, the phrase “about 70% by weight” comprises at least all values from 63% to 77% by weight. In another aspect, the term “about” means within 7% of a given value or range. For example, the phrase “about 70% by weight” comprises at least all values from 65% to 75% by weight.

Concentrations, amounts, percentages and other numerical values may be presented herein in a range format. It is to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range is explicitly recited.

### 1. Formulations

The bioavailability of orally administered therapeutic agents is classified according to the Biopharmaceutical Classification System (BCS), a guidance provided by the U.S. Food and Drug Administration (FDA) that classifies drug substances based on their aqueous solubility and intestinal permeability. This system allows an estimation of the effect that the factors of dissolution, solubility and permeability will have on oral drug absorption. The effect of these factors on oral drug absorption is highly important, since 85% of the highest selling drugs in the USA and Europe are orally administered. According to the BCS system, BCS Class I drugs are those agents that are highly permeable and soluble, being well absorbed with an absorption rate usually higher than the excretion rate. BCS Class II drugs are highly permeable but have low solubility, with bioavailability being limited by either or both aqueous solubility or dissolution rate. In some cases, a correlation can be made between the in vivo bioavailability and the in vitro dissolution rate for BCS II agents. BCS Class III drugs are highly soluble, but have low permeability. While a drug may be rapidly dissolved, absorption may be conversely limited by the permeation rate. If the formulation does not change the permeability or gastrointestinal (GI) transit time, then Class I criteria can be applied. BCS Class IV drugs have low permeability and solubility, with poor bioavailability, either not being well absorbed or having highly variable absorption over the intestinal mucosa. The BCS class boundary defines a drug as highly soluble when the highest dose strength is soluble in less than 250 mL of water over a pH range of 1 to 7.5 and to be highly permeable when the extent of absorption in humans is determined to be greater than 90% of an administered dose, based on mass-balance or in comparison to an intravenous reference dose. A drug product is considered to be rapidly dissolving when greater than 85% of the labeled amount of drug substance dissolves within 30 minutes using a USP apparatus I or II in a volume of less than 900 mL buffer solution.

The poor solubility and resulting poor bioavailability of a BCS II agent becomes a significant pharmaceutical development challenge since poorly water-soluble agents tend to be eliminated from the GI tract before being absorbed into circulation. Since BCS II agents dissolve poorly in the stomach and GI tract, they also tend to show a significant difference in their bioavailability and resulting plasma concentration depending on the presence or absence of food (generally referred to herein as the “food effect”), i.e., whether the subject is in a fed or fasted state when the agent is orally administered.

For example, the absorption of the agent may be significantly higher when the agent is administered after a meal than when the subject has not eaten prior to administration. Without being bound by theory, the different absorption pharmacokinetics of a fed or fasted state may be attributed to either or both the higher solubility of the lipophilic compound in fat or solubilization aided by bile salts that are secreted as a result of food intake. While this pharmacokinetic effect may be minimized in the absence of food, the resulting plasma concentration of the agent in the presence of food may lead to higher than expected plasma concentrations. For a drug with a narrow therapeutic and toxicity window this would not be desirable. Conversely, in the absence of food, the desired therapeutic plasma concentration may not be achieved. Accordingly, the food effect presents a significant regulatory hurdle to FDA approval for a BCS II agent and must be addressed in early drug development.

Ideally, to minimize the food effect, to maintain a consistent plasma level and to attain a desired therapeutic effect, the formulation of a BCS II agent must enhance the aqueous solubility of the lipophilic agent and must minimize the food effect. Formulation approaches designed to enhance the aqueous solubility of BCS II agents may involve a combination of pharmaceutically acceptable organic solvents or cosolvents, surfactants and modulation of pH conditions. While examples of such formulations exist, they often have some shortcomings with respect to gastric tolerances.

Also, depending on the ability of the formulation to balance the lipophilicity of the agent with the need for hydrophilicity, traditional formulations often cannot accommodate a sufficient quantity (dose load) of the agent in a dosage form that is convenient for oral administration. For example, a subject may be required to consume numerous units of the dosage form to obtain a plasma concentration of the lipophilic agent that provides the desired therapeutic effect. Formulations with this type of limitation thus discourage compliance with a required dosage regimen.

Several techniques for increasing the solubility of lipophilic agents, apart from enhancing the formulation, include identifying and selecting more soluble polymorphs, hydrates or salts of the agent.

Other techniques include using particle size reduction (i.e., micronization or nanoparticulate systems) to increase the molecular surface area of the dissolving solid in contact with the medium, thus accelerating dissolution and the potential for bioavailability. Micronization and other particle engineering approaches may include fine grinding of the crystalline form of the agent, precipitating a very fine form of the agent from solution, or forming a smaller particle or an amorphous form either by spray drying or freeze-drying the agent from a solution. Certain techniques for size reduction reduce the naturally-occurring or micronized particle size of the agent to a much greater extent, producing nanoparticles up to 1000 times smaller than the original. Certain other techniques coat the agent onto small particles to form a dispersion.

There also exist solubilization approaches based on the use of a solvent system, whereby solubilizing agents “drag” the BCS II agent into solution and increase the miscibility of the agent with aqueous media. These and other techniques known to those skilled in the art may be combined with different crystalline forms of the agent (e.g., an amorphous form) or eutectic mixtures to reduce the thermodynamic barriers to dissolution.

While these solubilization techniques focus on the agent's structure, crystalline form and particle size, their usefulness in producing a bioavailable oral dosage form depends on numerous factors related to the agent's molecular interactions with excipients in a pharmaceutical formulation. The effect of agent related factors on the usefulness of these solubilization techniques requires significant evaluation, often showing that the techniques alone may sometimes be inadequate to achieve the desired result of providing or improving satisfactory solubility and dose loading in the final formulation or that a compromised result for marketability is all that can be obtained.

For example, a common problem these techniques have in achieving the desired result of improved solubility is that, after the formation of an agent having a reduced particle size, a physical property of the very small particles is their tendency to agglomerate together and impede powder flow. Although solubility of the BCS II agent may be increased from the reduced particle size, the convenient and practical usability of the agent in a bulk form is reduced. One of the many techniques most often used in the field to reduce or prevent agglomeration though, coating the particles, adds another step to the formulation process.

An alternative method used to increase the surface area of a BCS II agent without the use of either micronization or nanoparticles, and thus provide or improve the solubility of the agent, is to make a solid dispersion of the agent in a suitable high molecular weight water-soluble polymeric matrix. A solid dispersion contains at least two components: a matrix and a molecular dispersion of an active agent within the matrix. The agent (as either crystalline or amorphous particles, optionally micronized or nanoparticles) is uniformly dispersed within the polymer matrix. Such a formulation provides a solubility bridge between the insoluble agent and an aqueous medium (e.g., GI fluid) and improves the dissolution properties of the agent when exposed to the medium.

Without being bound by theory, the molecular interactions between an aqueous solvent and the agent, when the agent is uniformly dispersed within the matrix, improve the solubility of the agent. Without limitation, solid dispersions may be physically classified as a eutectic mixture, a solid solution, a glass solution or suspension, an amorphous precipitate in a glassy or crystalline carrier, a complex, a complexed formation or a combination of the different systems. In addition, solid dispersion dosage forms may be formulated using various techniques well known to those skilled in the art, such as by co-dissolving the agent and polymer in a solvent then spray-drying, spray-congealing, evaporation, curing or microwaving, blending and direct compression, mechanical admixture at an elevated but non-melting temperature, wet granulation, extrusion-spheronization, melt fusion, hot melt extrusion and the like.

With the proper choice of one or more polymers, the solubility of both the BCS II agent and the resulting formulation may be significantly increased. Polymers such as, but not limited to, polyvinyl pyrrolidone (PVP) are commonly used to form a polymeric matrix with an agent

A typical method of preparing a solid dispersion includes co-dissolving the polymer and the agent in a solvent. The materials may form a suspended or an unsaturated mixture or a saturated or supersaturated matrix-solvent mixture. The solvent is then removed to leave a complexed mixture of agent and polymer as a matrix. Without limitation, solvent removal methods include precipitation, freeze-drying, vacuum drying or spray drying. However, identifying a common solvent or solvent system that effectively dissolves the agent and the matrix requires substantial evaluation.

For example, if the chemical requirements of the polymer and agent require the amount of solvent used to co-dissolve them to be large, the process of removing the solvent becomes expensive and impractical. Although suitable solvents may be found at a suitable volume, those considered suitable by a formulator may be regarded by the FDA as toxic, which renders them impractical for pharmaceutical use. Alternatively, using surfactants and solubilizing agents to reduce the amount of solvent used can lead to insufficient loading of the agent in the dosage form and high concentrations of surfactants. Such changed properties of the formulation may be commercially unviable at best or poorly-tolerated or even toxic at worst.

Although the solid dispersion technique has been used to improve the solubility of a number of marketed BCS II agents for pharmaceutical use, the ability to effectively implement the technique has been hampered by the need for solid dispersion formulations that form a physically and chemically stable mixture between the polymer matrix and agent when in solution and also in the solid state after formation of the matrix.

For example, a polar polymeric matrix may enhance dissolution but, when the polar polymer is co-dissolved with a lipophilic agent, the materials may be inherently prone to phase separation. This tendency can be magnified if the polar polymer is also hygroscopic. The result in both cases is reduced physical stability. Conversely, a stable matrix that prevents phase changes of the agent within the matrix requires low molecular mobility. The polymer that provides low molecular mobility is usually of a high molecular weight, which increases the difficulty of finding a common solvent for both agent and polymer. If the matrix is made using a less polar polymer in order to more easily find a common solvent, then the dissolution rate could be impaired. Moreover, it would be highly desirable to find an optimum solid dispersion formulation combined with a viable commercial production process.

Despite many years of research and development and despite its theoretical promise, the practical application of the solid dispersion approach has been limited by the need to use trial and error to develop a specific matrix for a specific agent because the interaction of the agent and polymer in forming the polymeric matrix are not scientifically understood. Additionally, many of the requirements built into such a formulation are mutually incompatible, including the low hygroscopicity of the BCS II agent combined with a polymer having high hygroscopicity, the need for fast dissolution while maintaining long-term physical and chemical stability of the agent-polymer matrix, the need for ease of commercial manufacture when scaling up the solid dispersion.

For example, International Patent Publication WO2005/084639 describes formulations that contain a Class II drug having low oral bioavailability, together with a hydrophobic polymer co-dissolved in a common solvent wherein the solution is formed into small solid particles and dispersed in a polymeric matrix. Enhancement of bioavailability occurs through increased dissolution kinetics due to stable micronization and rapid release from the polymer in the GI tract.

In another example, United States Patent Publication US2009/0098200 describes solid dispersions comprising a poorly soluble bioactive compound dispersed and characterized in a polymer matrix which may comprise more than one polymer.

Although numerous and different methods have been proposed for preparing formulations for poorly soluble agents, the feasibility for use of any of the proposed formulations requires substantial evaluation for each agent.

Accordingly, there remains a continuing need in the art and a continuing demand in the market for pharmaceutical compositions having ease of dosing with increased dose loading and improved dissolution less subject to the food effect that are useful for a particular agent.

Formulations may be prepared using a pharmaceutically acceptable carrier composed of materials that are considered safe and effective and may be administered to an individual without causing undesirable biological side effects or unwanted interactions.

As used herein, the term “carrier” refers to all components present in the pharmaceutical formulation other than the active ingredient and includes, but is not limited to, diluents, binders, lubricants, disintegrants, stabilizers, surfactants, colorants or fillers.

Solid dispersions, such as spray dried intermediates of Compound 1 provided herein can be administered to a patient orally or parenterally in the conventional form of preparations, such as capsules, microcapsules, tablets, granules, powder, troches, pills, suppositories, suspensions and syrups. Suitable formulations can be prepared by methods commonly employed using conventional, organic or inorganic additives, such as an excipient selected from fillers or diluents, binders, disintegrants, lubricants, flavoring agents, preservatives, stabilizers, suspending agents, dispersing agents, surfactants, antioxidants or solubilizers.

In one aspect, the spray dried intermediate of Compound 1 provided herein is administered orally using a capsule dosage form composition, wherein the capsule contains a spray dried intermediate of Compound 1 provided herein with or without an additional carrier, excipient or vehicle. Capsules can be prepared by mixing the spray dried intermediate of Compound 1 provided herein with a suitable carrier or diluent and filling the proper amount of spray dried intermediate of Compound 1 or mixture in the capsules.

In another aspect, provided herein are compositions comprising an effective amount of Compound 1 provided herein and a pharmaceutically acceptable carrier or vehicle, wherein a pharmaceutically acceptable carrier or vehicle can comprise an excipient, diluent, or a mixture thereof. Compositions can be formulated to contain a daily dose, or a convenient fraction of a daily dose, in a dosage unit. In general, the composition is prepared as a tablet according to known methods.

In one aspect, the composition is a pharmaceutical composition.

The pharmaceutical composition described herein comprises a spray dried intermediate in intimate admixture with one or more pharmaceutically acceptable excipients to provide a bioavailable oral dosage form.

The spray dried intermediate described herein comprises an amorphous form of Compound 1 and a polymer.

In one aspect, the form of Compound 1 used to prepare the intermediate is an amorphous polymorph form. The advantage of the amorphous form lies in certain properties that make the form amendable for use in a dry blend with additional excipients. The advantageous amorphous form properties include a reduced particle size, increased particle distribution and better flow characteristics, dispersion and content uniformity in the final dosage form.

An amorphous form described herein may be prepared using a variety of methods known to those skilled in the art. The techniques for preparing an amorphous form are well known in the art and are described herein. Spray drying was the technique selected to prepare the spay dried intermediate comprising the amorphous form of the Compound and a polymer.

In one aspect, the form of Compound 1 used to prepare the spray dried intermediate is a crystalline form. The advantage of the crystalline form lies in more efficient manufacture of the intermediate, wherein a liquid dispersion comprising the crystalline form, the polymer and optional excipients are co-dissolved in a solvent system to form a liquid dispersion.

The solvent system used may comprise one or more solvents in certain ratios, wherein the solvent ratio provides an optimum process for dissolving Compound 1 and polymer to prepare the liquid dispersion. The optimum mixture and ratio of solvents in a solvent system depend on a balance of dose loading and the amount and type of polymer used, in particular the molecular weight of the polymer.

Aspects described herein include one or more solvents selected from THF (tetrahydrofuran), MeOH (methanol), EtOH (ethanol), acetone, EtOH-95 (ethanol at 95% proof), absolute EtOH (ethanol at 99.99% proof), DCM (dichloromethane), IPA (isopropanol), DMSO (dimethylsulfoxide), DMF (dimethylformamide), water or mixtures thereof.

An aspect of a solvent system for use as described herein may comprise DCM in a mixture with at least one other solvent. One aspect of a solvent system comprising DCM in a mixture with at least one other solvent for use as described herein may comprise a solvent system selected from DCM:acetone, DCM:DMSO, DCM:EtOH-95, DCM:EtOH-absolute, DCM:IPA, DCM:MeOH or DCM:THF. Certain aspects may comprise a mixture of solvents selected from DCM:DMSO or DCM:MeOH.

The amount of DCM used in a solvent system mixture as described herein includes an amount of about 5%, 10%, 15%, 20%, 25%, 30%, 35%, 40%, 45%, 50%, 55%, 60%, 65%, 70%, 75%, 80%, 85%, 90%, 95% or 100%. In one aspect, the amount of DCM used in a solvent system mixture as described herein includes an amount in a range of from about 5% to about 10%, from about 10% to about 15%, from about 15% to about 20%, from about 20% to about 25%, from about 25% to about 30%, from about 30% to about 35%, from about 35% to about 40%, from about 40% to about 45%, from about 45% to about 50%, from about 50% to about 55%, from about 55% to about 60%, from about 60% to about 65%, from about 65% to about 70%, from about 70% to about 75%, from about 75% to about 80%, from about 80% to about 85%, from about 85% to about 90%, from about 90% to about 95% or from about 95% to about 100%.

The amount of the other solvent used in a solvent system mixture with DCM as described herein includes an amount of about 0%, 5%, 10%, 15%, 20%, 25%, 30%, 35%, 40%, 45%, 50%, 55%, 60%, 65%, 70%, 75%, 80%, 85%, 90% or 95%. In one aspect, the amount of the other solvent used in a solvent system mixture with DCM as described herein includes an amount in a range of from about 0% to about 5%, from about 5% to about 10%, from about 10% to about 15%, from about 15% to about 20%, from about 20% to about 25%, from about 25% to about 30%, from about 30% to about 35%, from about 35% to about 40%, from about 40% to about 45%, from about 45% to about 50%, from about 50% to about 55%, from about 55% to about 60%, from about 60% to about 65%, from about 65% to about 70%, from about 70% to about 75%, from about 75% to about 80%, from about 80% to about 85%, from about 85% to about 90% or from about 90% to about 95%.

In one aspect, the amount of DCM used in a solvent system as described herein includes an amount in a range of from about 10% to about 100%, an amount in a range of from about 30% to about 87% or an amount in a range of from about 50% to about 86%, an amount in a range of from about 33% to about 87%, an amount in a range of from about 65% to about 87%.

In one aspect, the amount of the other solvent used in a solvent system mixture with DCM as described herein includes an amount in a range of from about 0% to about 100%, an amount in a range of from about 5% to about 13% or an amount in a range of from about 50% to about 86%, an amount in a range of from about 33% to about 87.5%, an amount in a range of from about 65% to about 87.5%.

In one aspect, the ratio of the amount of DCM and the other solvent used in a solvent system mixture with DCM (wherein the ratio is expressed as DCM:solvent), as described herein include ratios for DCM:acetone, DCM:DMSO, DCM:EtOH-95, DCM:EtOH-absolute, DCM:IPA, DCM:MeOH or DCM:THF.

In one aspect, the ratio of DCM:acetone may be about 50:50. In one aspect, the ratio of DCM:DMSO may be about 50:50, about 65:35, about 77:23, about 80:20, or about 95:5. In one aspect, the ratio of DCM:EtOH may be about 80:20. In one aspect, the ratio of DCM:EtOH-95 may be about 50:50, about 80:20, about 86:14, 87:13 or about 87.5:12.5. In one aspect, the ratio of DCM:IPA may be about 50:50. In one aspect, the ratio of DCM:MeOH may be about 50:50, about 80:20, about 86:14, 87:13 or about 87.5:12.5. In one aspect, the ratio of DCM:THF may be about 33:67.

Another factor in the design of an optimum solvent system includes the amount and type of excipients used, in particular excipients that affect the amphiphilic character of the polymer and corresponding spray dried intermediate polymer matrix, wherein the hydrophobic and hydrophilic interaction of the polymer with Compound 1 is ultimately affected in the gastric environment.

In one aspect, the polymer used is a hydrophilic polymer.

One of the factors influencing the release of drugs from hydrophilic matrices include viscosity of the polymer, ratio of the polymer to drug, mixtures of polymers, compression pressure, thickness of the tablet, particle size of the drug, pH of the matrix, entrapped air in the tablets, molecular size of the drug, molecular geometry of the drug, solubility of the drug, the presence of excipients or additives, and the mode of incorporation of these substances (Patel V F, Patel N M. Statistical Evaluation of Influence of Viscosity and Content of Polymer on Dipyridamole Release From Floating Matrix Tablets: A Technical Note. AAPS PharmSciTech. 2007; 8(3): Article 69).

In one aspect, the polymer used is selected from PVP.

Also encompassed is a method for preparing a solid dispersion comprising an amorphous form of Compound 1 and the polymer.

The solid dispersion may be fabricated using any of the matrix formation methods known to those skilled in the art, including but not limited to: solvent evaporation, solvent removal, spray-drying, phase inversion encapsulation, spontaneous emulsification, coacervation, hot melt encapsulation, hot melt extrusion, spray-congealing, prilling and grinding. It is understood that the solid dispersion may be further processed into an oral dosage form using any of the standard pharmaceutical techniques including, but not limited to, tabletting, extrusion-spheronization and fluidized bed coating for multiparticulate dosage forms and capsule-filling.

Although the primary source of adhesiveness and of prevention of aggregation is the nature of the polymer(s) forming the matrix and methods of preparation are known to those skilled in the art, a significant amount of evaluation is required to prepare the polymeric matrix of a solid dispersion comprising the amorphous form of Compound 1 and a polymer.

In one aspect, the method includes co-dissolving Compound 1 and the polymer in a solvent system to form a liquid dispersion then removing the solvent.

In one aspect, the intermediate formed is a solid dispersion.

In one aspect, the method of forming the solid dispersion herein includes removing the solvent by a suitable means, including spray drying a solution containing a dissolved polymer and dispersed fine particles of Compound 1. Another method involves dissolving a polymer and dissolving or suspending a Compound and then diluting the solution with a large volume of an anti-solvent for the polymer and Compound 1, where the solvent is substantially miscible with the anti-solvent.

In one aspect, the solution comprises a Compound-polymer mixture co-dissolved in a mutual solvent and then spray-dried to form microparticles. The polymer system acts as a matrix for more rapid dissolution of Compound 1 due to increased surface area by maintaining the micronized Compound particle size.

The resulting spray dried intermediate (SDI) is then incorporated with suitable pharmaceutical excipients for use in preparing a tablet or capsule dosage form for oral administration.

Dose loading of Compound 1 in the spray-drying solution can range from about 1% to about 90% (w/w), from about 1% to about 50% w/w, from 20% to about 70% w/w, from 20% to about 60% w/w, from 30% to about 40% w/w or from about 20% to about 30% w/w.

In one aspect, the amorphous form of Compound 1 is formed as the spray dried intermediate is obtained.

The use of the spray dried intermediate in a pharmaceutical composition comprising the intermediate in intimate admixture with one or more pharmaceutically acceptable excipients to provide a bioavailable oral dosage form is also described.

### Excipients

The formulation may include one or more excipients. Suitable excipients include solvents, co-solvents, emulsifiers, plasticizers, surfactants, thickeners, pH modifiers, emollients, antioxidants, and chelating agents, wetting agents, and water absorbing agents. The formulation may also include one or more additives, for example, dyes, colored pigments, pearlescent agents, deodorizers, and odor maskers.

Other suitable excipients that may be selected are known to those skilled in the art and include, but are not limited to fillers or diluents (e.g., sucrose, starch, mannitol, sorbitol, lactose, glucose, cellulose, talc, calcium phosphate or calcium carbonate and the like), a binder (e.g., cellulose, carboxymethylcellulose, methylcellulose, hydroxymethylcellulose, hydroxypropylmethylcellulose, polypropylpyrrolidone, polyvinylpyrrolidone, gelatin, gum arabic, polyethyleneglycol or starch and the like), a disintegrant (e.g., sodium starch glycolate, croscarmellose sodium and the like), a lubricant (e.g., magnesium stearate, light anhydrous silicic acid, talc or sodium lauryl sulfate and the like), a flavoring agent (e.g., citric acid, or menthol and the like), a preservative (e.g., sodium benzoate, sodium bisulfite, methylparaben or propylparaben and the like), a stabilizer (e.g., citric acid, sodium citrate or acetic acid and the like), a suspending agent (e.g., methylcellulose, polyvinyl pyrrolidone or aluminum stearate and the like), a dispersing agent (e.g., hydroxypropylmethylcellulose and the like), surfactants (e.g., sodium lauryl sulfate, polaxamer, polysorbates and the like), antioxidants (e.g., ethylene diamine tetraacetic acid (EDTA), butylated hydroxyl toluene (BHT) and the like) and solubilizers (e.g., polyethylene glycols, SOLUTOL®, GELUCIRE® and the like). The effective amount of Compound 1 provided herein in the pharmaceutical composition may be at a level that will exercise the desired effect.

Diluents, also referred to herein as “fillers”, are typically necessary to increase the bulk of a solid dosage form so that a practical size is provided for compression of tablets or formation of beads and granules. Suitable diluents include, but are not limited to, dicalcium phosphate dehydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

Dispersants include, among others water, phosphate-buffered saline (PBS), saline, glucose, sodium lauryl sulfate (SLS), polyvinylpyrrolidone (PVP), polyethylene glycol (PEG), and hydroxypropylmethylcellulose (HPMC Binders are used to impart cohesive qualities to a solid dosage formulation, and thus ensure that a tablet, bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose (“HPMC”), micro crystalline cellulose (“MCC”) , hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone (PVP).

Lubricants used to facilitate tablet manufacture. Examples of suitable lubricants include, but are not limited to, magnesium stearate, calcium stearate, stearic acid, glycerol behenate, polyethylene glycol, talc, sodium stearyl fumarate, fumed silica and mineral oil.

Disintegrants are used to facilitate dosage form disintegration or; “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross linked PVP (Crospovidone, POLYPLASDONE XL), croscarmellose sodium.

Stabilizers are used to inhibit or retard active ingredient decomposition reactions which include, by way of example, oxidative reactions.

Surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2 ethylthioxyl)-sulfosuccinate, and alkyl sulfates such as sodium lauryl sulfate.

Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, I Poloxamer 401, stearoyl monoisopropanolamide, and polyoxyethylene hydrogenated tallow amide. Examples of amphoteric surfactants include sodium N-dodecyl-p-alanine, sodium N-lauryl-iminodipropionate, myristoamphoacetate, lauryl betaine and lauryl sulfobetaine.

If desired, the tablets, beads, granules, or particles may also contain minor amount of nontoxic auxiliary substances such as wetting or emulsifying agents, dyes, pH buffering agents, or preservatives.

The formulation may be in the form of a tablet, capsule, minitab, filled tablet, osmotic device, slurry, dispersion, or suspension. In a preferred aspect, the formulation is a solid oral dosage formulation, such as a tablet, multiparticulate composition, or capsule.

Compound 1 may be administered in a formulation wherein a spray dried intermediate comprising Compound 1 in amorphous form and a hydrophylic polymer, such as PVP, is in an admixture with one or more pharmaceutically acceptable carriers, excipients or diluents. The pharmaceutical formulations may be produced using standard procedures known to those skilled in the art.

### Immediate Release

In one aspect, the composition is included in an immediate release formulation. Preferably Compound 1 is in the form of microparticles of spray dried intermediates comprising an amorphous form of Compound 1 and a polymer. The microparticles are stabilized against aggregation by the polymer; therefore, any of the standard tablet, or capsule oral dosage forms may be used. The microparticles may be further formulated into tablets, slurries or dispersions for oral administration or placed in capsules, such as gelatin capsules.

The matrix of polymer of the spray dried intermediate is preferably porous, or otherwise allows ready dissolution of Compound 1 in the fluids of the gastrointestinal tract. This allows rapid dissolution of Compound 1 without reduction in effective particle area by agglomeration of undissolved particles. A matrix that is bioadhesive further enhances absorption by tending to retain the particles in the stomach or upper intestine while the Compound is absorbed.

### Controlled Release

The delayed release/extended release pharmaceutical compositions can be obtained by complexing the SDI with a pharmaceutically acceptable ion exchange resin and coating such complexes. The SDI is coated with a substance that will act as a barrier to control the diffusion of Compound 1 from its core complex into the gastrointestinal fluids. Optionally, the SDI is coated with a polymer film which is insoluble in the acid environment of the stomach, and soluble in the basic environment of lower GI tract in order to obtain a final dosage form that releases less than 10% of the dose load within the stomach.

Examples of suitable controlled release coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, polysaccharides, acrylic acid polymers and copolymers, or a methacrylic resin.

Additionally, the coating material may contain conventional carriers; such as plasticizers, pigments, colorants, glidants, stabilization agents, pore formers, and surfactants.

Accordingly, the release rate may be altered by coating a tablet with sugars, enteric polymers or gelatin to alter dissolution of the tablet. Premature dissolution of the tablet in the mouth may be prevented by coating with hydrophilic polymers, such as HPMC (of a grade having an increased polymer length and higher viscosity) or gelatin, resulting in dissolution in the stomach.

The composition can also be designed to extend the time period for release by increasing Compound 1 to carrier ratio, with release drawn out to about 80% in about 90 minutes (in vitro). Increased relative Compound concentration is believed to have the effect of increasing the effective drug domain size within a polymer matrix. The increased drug domain size results in slower drug dissolution. In the case of a polymer matrix containing certain types of hydrophilic polymers, the polymer will act as a mucoadhesive material and increase the retention time of the drug in the gastrointestinal tract. Increased drug dissolution rates combined with the mucoadhesive properties of the polymer matrix results in (1) increased uptake of the drug and (2) reduction in differences found in the fed and fasted states for BCS Class II drugs.

The oral dosage formulations described herein can be used to treat a variety of diseases and disorders. These formulations have improved bioavailability over formulations that do not contain the bioadhesive polymers.

The formulations are designed to facilitate diffusion of drug into intestinal tissue. the formulations can be designed to release drug slowly, quickly or in a step-wise (pulsatile) manner.

Accordingly, the present application provides pharmaceutical compositions having increased dose loading and improved solubility not subject to the effect of food.

### Methods of Use

Also encompassed herein are methods for treating conditions described herein. In one aspect, the methods for treating a condition described herein involve the administration of Compound 1, as a single agent therapy, to a patient in need thereof. In a specific aspect, presented herein is a method for treating a condition described herein, comprising administering to a patient in need thereof an effective amount of Compound 1, as a single agent. In another aspect, presented herein is a method for treating a condition described herein, comprising administering to a patient in need thereof a pharmaceutical composition comprising Compound 1, as the single active ingredient, and a pharmaceutically acceptable carrier, excipient or vehicle.

In another aspect, the methods for treating a condition described herein involve the administration of Compound 1 in combination with another therapy to a patient in need thereof. Such methods may involve administering Compound 1 prior to, concurrent with, or subsequent to administration of the additional therapy. In certain aspects, such methods have an additive or synergistic effect. In a specific aspect, presented herein is a method for treating a condition described herein, comprising administering to a patient in need thereof an effective amount of Compound 1 and an effective amount of another therapy.

In specific aspects, any condition that is amenable to inhibition of the production of DHODH can be treated in accordance with the methods provided herein. In another specific aspect, the condition treated in accordance with the methods provided herein is a leukemia selected from the group consisting of an acute or chronic leukemia, wherein the acute leukemia is selected from acute lymphocytic leukemia, acute myelocytic leukemias such as myeloblastic, promyelocytic, myelomonocytic, monocytic, erythroleukemia leukemias or myclodysplastic syndrome; and, wherein the chronic leukemia is selected from chronic myclocytic (granulocytic) leukemia, chronic lymphocytic leukemia, hairy cell leukemia; or polycythemia vera; and, the like

### Dosage and Administration

In accordance with the methods for treating a condition described herein, a Compound or a pharmaceutical composition thereof can be administered to a subject in need thereof by a variety of routes in amounts which result in a beneficial or therapeutic effect. Compound 1 or a pharmaceutical composition thereof may be orally administered to a subject in need thereof in accordance with the methods for treating a condition described herein. The oral administration of Compound 1 or a pharmaceutical composition thereof may facilitate subjects in need of such treatment complying with a regimen for taking the Compound or pharmaceutical composition. Thus, in a specific aspect, Compound 1 or pharmaceutical composition thereof is administered orally to a subject in need thereof.

Pharmaceutical compositions or forms of Compound 1 provided herein can be administered orally, with or without food or water.

Other routes of administration include, but are not limited to, intravenous, intradermal, intrathecal, intramuscular, subcutaneous, intranasal, inhalation, transdermal, topical, transmucosal, intracranial, intratumoral, epidural and intra-synovial. In one aspect, Compound 1 or a pharmaceutical composition thereof is administered systemically (e.g., parenterally) to a subject in need thereof. In another aspect, Compound 1 or a pharmaceutical composition thereof is administered locally (e.g., intratumorally) to a subject in need thereof. In one aspect, Compound 1 or a pharmaceutical composition thereof is administered via a route that permits the Compound to cross the blood-brain barrier (e.g., orally).

In accordance with the methods for treating a condition described herein that involve administration of Compound 1 in combination with one or more additional therapies, the Compound and one or more additional therapies may be administered by the same route or a different route of administration.

The dosage and frequency of administration of Compound 1 or a pharmaceutical composition thereof is administered to a subject in need thereof in accordance with the methods for treating a condition described herein will be efficacious while minimizing any side effects. The exact dosage and frequency of administration of Compound 1 or a pharmaceutical composition thereof can be determined by a practitioner, in light of factors related to the subject that requires treatment. Factors which may be taken into account include the severity of the disease state, general health of the subject, age, weight, and gender of the subject, diet, time and frequency of administration, drug combination(s), reaction sensitivities, and tolerance/response to therapy. The dosage and frequency of administration of Compound 1 or a pharmaceutical composition thereof may be adjusted over time to provide sufficient levels of the Compound or to maintain the desired effect.

In certain aspects, Compound 1 or a pharmaceutical composition thereof is administered to a subject in accordance with the methods for treating a condition described herein once a day, twice a day, three times a day, or four times a day. In some aspects, Compound 1 or a pharmaceutical composition thereof is administered to a subject in accordance with the methods for treating a condition described herein once, twice, three times, or four times every other day (i.e., on alternate days), once, twice, three times, or four times every two days, once every three days, once, twice, three times, or four times every four days, once, twice, three times, or four times every 5 days, once, twice, three times, or four times a week, once, twice, three times, or four times every two weeks, once, twice, three times, or four times every three weeks, once, twice, three times, or four times every four weeks, once, twice, three times, or four times every 5 weeks, once, twice, three times, or four times every 6 weeks, once, twice, three times, or four times every 7 weeks, or once, twice, three times, or four times every 8 weeks. In particular aspects, Compound 1 or a pharmaceutical composition thereof is administered to a subject in accordance with the methods for treating a condition described herein in cycles, wherein the Compound or pharmaceutical composition is administered for a period of time, followed by a period of rest (i.e., the Compound or pharmaceutical composition is not administered for a period of time).

In certain aspects, Compound 1 or a pharmaceutical composition thereof is administered to a subject in need thereof in accordance with the methods for treating a neoplasm provided herein at a dosage and a frequency of administration that achieves one or more of the following: (i) decreases the production and/or concentration of DHODH or other angiogenic or inflammatory mediators or a change in tumor blood flow or metabolism, or peritumoral inflammation or edema of a subject with a condition described herein or an animal model with a pre-established human tumor; (ii) decreases the concentration of one, two, three or more, or all of the following of a subject with a neoplasm or an animal model with a pre-established human tumor: DHODH; (iii) reduces or ameliorates the severity of the neoplasm and/or one or more symptoms associated therewith in a subject with the neoplasm; (iv) reduces the number symptoms and/or the duration of one or more symptoms associated with the neoplasm in a subject with the neoplasm; (v) prevents the onset, progression or recurrence of one or more symptoms associated with the neoplasm in a subject with the neoplasm or an animal model with a pre-established human tumor; (vi) reduces the size of the tumor in a subject with the neoplasm or in an animal model with a pre-established human tumor; (vii) reduces angiogenesis associated with a malignant neoplasm in a subject or an animal model with a pre-established human tumor; and/or (vii) enhances or improves the therapeutic effect of another therapy in a subject with the neoplasm or an animal model with a pre-established human tumor.

In certain aspects, Compound 1 or a pharmaceutical composition thereof is administered to a subject in need thereof in accordance with the methods for treating a neoplastic or non-neoplastic condition provided herein at a dosage and a frequency of administration that results in one or more of the following: (i) a decrease in the number of circulating tumor cells (CTCs) in the blood of a subject with a neoplastic or non-neoplastic condition or an animal model with a pre-established human tumor; (ii) a decrease in circulating DNA or RNA associated with tumor cells in the blood of a subject having a condition; (iii) survival of patients with a neoplastic or non-neoplastic condition for about 6 months or more, about 7 months or more, about 8 months or more, about 9 months or more, or about 12 months or more; (iv) regression of a tumor associated with a neoplastic condition and/or inhibition of the progression of a tumor associated with a neoplastic condition in a subject with a neoplastic condition or an animal model with a pre-established human tumor; (v) reduction in the growth of a neoplasm and/or decrease in the tumor size (e.g., volume, cross-sectional area or diameter) of tumors associated with the neoplasm in a subject with a neoplasm or an animal model with a pre-established human tumor; (vi) the size of a tumor associated with a neoplasm is maintained and/or the tumor does not increase or increases by less than the increase of a similar tumor in a subject with a neoplasm or an animal model with a pre-established human tumor after administration of a standard therapy as measured by conventional methods available to one of skill in the art, such as digital rectal exam, ultrasound (e.g., transrectal ultrasound), CT Scan, PET scan, DCE-MRI, and MRI; (vii) reduction in the formation of a tumor associated with a neoplasm in a subject with the neoplasm or an animal model with a pre-established human tumor; (viii) the eradication, removal, or control of primary, regional and/or metastatic tumors associated with a neoplasm in a subject with the neoplasm or an animal model with a pre-established human tumor; (ix) a decrease in the number or size of metastases associated with a malignant neoplasm in a subject with the neoplasm or an animal model with a pre-established human tumor; (x) a reduction or inhibition of the recurrence of a tumor; (xi) a reduction in edema or inflammation associated with a tumor; (xii) an inhibition or reduction in tumor vascularization; (xiii) a reduction of pathologic angiogenesis; and/or (x) reduction in the growth of a pre-established tumor or neoplasm and/or decrease in the tumor size (e.g., volume, cross-sectional area or diameter) of a pre-established tumor in a subject with a malignant neoplasm or an animal model with a pre-established human tumor.

In certain aspects, Compound 1 or a pharmaceutical composition thereof is administered to a subject in need thereof in accordance with the methods for treating a non-neoplastic condition provided herein at a dosage and a frequency of administration that achieves one or more of the following: (i) decreases the production or concentration of DHODH or other angiogenic or inflammatory mediators; (ii) decreases the concentration of one, two, three or more, or all of the following of a subject with a non-neoplastic condition or an animal model: DHODH; (iii) reduces or ameliorates the severity of the non-neoplastic condition and/or one or more symptoms associated therewith in a subject with the non-neoplastic condition; (iv) reduces the number symptoms and/or the duration of one or more symptoms associated with the non-neoplastic condition in a subject with the non-neoplastic condition; (v) prevents the onset, progression or recurrence of one or more symptoms associated with the non-neoplastic condition in a subject with the non-neoplastic condition; (vi) reduces inflammation associated with the non-neoplastic condition; (vii) reduces pathologic angiogenesis associated with the non-neoplastic condition in a subject or an animal model; and/or (viii) enhances or improves the therapeutic effect of another therapy in a subject with the non-neoplastic condition or an animal model.

In one aspect, a method for treating a condition described herein involves the administration of a unit dosage of Compound 1 or a pharmaceutical composition thereof. The dosage may be administered as often as determined effective (e.g., once, twice or three times per day, every other day, once or twice per week, biweekly or monthly). In certain aspects, a method for treating a condition described herein involves the administration to a subject in need thereof of a unit dose of Compound 1 or a pharmaceutical composition thereof that ranges from about 0.1 milligram (mg) to about 30000 mg, from about 1 mg to about 10000 mg, from about 5 mg to about 1000 mg, from about 10 mg to about 500 mg, from about 100 mg to about 500 mg, from about 150 mg to about 500 mg, from about 150 mg to about 8000 mg, from about 250 mg to about 8000 mg, from about 300 mg to about 8000 mg, or from about 500 mg to about 8000 mg, or any range in between. In some aspects, a method for treating a condition described herein involves the administration to a subject in need thereof of a unit dose of a Compound or a pharmaceutical composition thereof of about 15 mg, 16, mg, 17 mg, 18 mg, 19 mg, 20 mg, 21, mg, 22 mg, 23 mg, 24 mg, 25 mg, 26 mg, 27 mg, 28 mg, 29 mg, 30 mg or 40 mg. In certain aspects, a method for treating a condition described herein involves the administration to a subject in need thereof of a unit dose of Compound 1 or a pharmaceutical composition thereof of about 50 mg, 60 mg, 70 mg, 80 mg, 90 mg, 100 mg, 110 mg, 120 mg, 125 mg, 130 mg, 140 mg, 150 mg, 175 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 1000 mg, 1600 mg, 3200 mg, 4000 mg, 4500 mg, 5000 mg, 5500 mg, 6000 mg, 7000 mg, 7500 mg, 8000 mg and the like.

In some aspects, a method for treating a condition described herein involves the administration to a subject in need thereof of a unit dose of Compound 1 or a pharmaceutical composition thereof of at least about 0.1 mg, 1 mg, 5 mg, 10 mg, 20 mg, 30 mg, 40 mg, 50 mg, 60 mg, 70 mg, 80 mg, 90 mg, 100 mg, 110 mg, 120 mg, 125 mg, 130 mg, 140 mg, 150 mg, 175 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 1000 mg, 1600 mg, 3200 mg, 4000 mg, 4500 mg, 5000 mg, 5500 mg, 6000 mg, 7000 mg, 7500 mg, 8000 mg and the like. In certain aspects, a method for treating a condition described herein involves the administration to a subject in need thereof of a unit dose of Compound 1 or a pharmaceutical composition thereof of less than about 35 mg, less than about 40 mg, less than about 45 mg, less than about 50 mg, less than about 60 mg, less than about 70 mg, or less than about 80 mg.

In specific aspects, a method for treating a condition described herein involves the administration to a subject in need thereof of a unit dose of Compound 1 or a pharmaceutical composition thereof of about 20 mg to about 500 mg, about 40 mg to about 500 mg, about 40 mg to about 200 mg, about 40 mg to about 150 mg, about 75 mg to about 500 mg, about 75 mg to about 450 mg, about 75 mg to about 400 mg, about 75 mg to about 350 mg, about 75 mg to about 300 mg, about 75 mg to about 250 mg, about 75 mg to about 200 mg, about 100 mg to about 200 mg, or any range in between. In other specific aspects, a method for treating a condition described herein involves the administration to a subject in need thereof of a unit dose of Compound 1 or a pharmaceutical composition thereof of about 20 mg, 35 mg, 40 mg, 50 mg, 60 mg, 75 mg, 100 mg, 125 mg, 150 mg, 175 mg, 200 mg, 225 mg, 250 mg or 300 mg. In some aspects, a method for treating a condition described herein involves the administration to a subject in need thereof of a unit dose of a Compound or a pharmaceutical composition thereof of about 350 mg, 400 mg, 500 mg, 600 mg, 700 mg, 800 mg, 900 mg, 1000 mg, 1600 mg, 3200 mg, 4000 mg, 4500 mg, 5000 mg, 5500 mg, 6000 mg, 7000 mg, 7500 mg, 8000 mg and the like. In some aspects, a unit dose of a Compound or a pharmaceutical composition thereof is administered to a subject once per day, twice per day, three times per day; once, twice or three times every other day (i.e., on alternate days); once, twice or three times every two days; once, twice or three times every three days; once, twice or three times every four days; once, twice or three times every five days; once, twice, or three times once a week, biweekly or monthly, and the dosage may be administered orally.

In certain aspects, a method for treating a condition described herein involves the oral administration to a subject in need thereof of a unit dose of a Compound1 or a pharmaceutical composition thereof that ranges from about 20 mg to about 500 mg per day. In some aspects, a method for treating a condition described herein involves the oral administration to a subject in need thereof of a unit dose of Compound 1 or a pharmaceutical composition thereof that ranges from about 80 mg to about 500 mg per day, about 100 mg to about 500 mg per day, about 80 mg to about 400 mg per day, about 80 mg to about 300 mg per day, about 80 mg to about 200 mg per day, about 200 mg to about 300 mg per day, about 200 mg to about 400 mg per day, about 200 mg to about 500 mg per day, or any range in between.

In a specific aspect, a method for treating a condition described herein involves the oral administration of a unit dose of about 200 mg of Compound 1 or a pharmaceutical composition thereof once per day. In another specific aspect, a method for treating a condition described herein involves the oral administration to a subject in need thereof of a unit dose of about 100 mg of Compound 1 or a pharmaceutical composition thereof twice per day. In another specific aspect, a method for treating a condition described herein involves the oral administration of a unit dose of about 50 mg of Compound 1 or a pharmaceutical composition thereof four times per day. In specific aspects, a method for treating a condition described herein involves the oral administration to a subject in need thereof of a unit dose of about 100 mg to about 250 mg, about 150 mg to about 250 mg, about 175 mg to about 250 mg, about 200 mg to about 250 mg, or about 200 mg to about 225 mg of Compound 1 or a pharmaceutical composition thereof twice per day.

In some aspects, a method for treating a condition described herein involves the administration of a dosage of Compound 1 or a pharmaceutical composition thereof that is expressed as mg per meter squared (mg/m²). The mg/m² for a Compound may be determined, for example, by multiplying a conversion factor for an animal by an animal dose in mg per kilogram (mg/kg) to obtain the dose in mg/m² for human dose equivalent. For regulatory submissions the FDA may recommend the following conversion factors: Mouse=3, Hamster=4.1, Rat=6, Guinea Pig=7.7. (based on Freireich et al., Cancer Chemother. Rep. 50(4):219-244 (1966)). The height and weight of a human may be used to calculate a human body surface area applying Boyd's Formula of Body Surface Area. In specific aspects, a method for treating a condition described herein involves the administration to a subject in need thereof of an amount of a Compound or a pharmaceutical composition thereof in the range of from about 0.1 mg/m² to about 1000 mg/m², or any range in between.

Other non-limiting exemplary doses of Compound 1 or a pharmaceutical composition that may be used in the methods for treating a condition described herein include mg amounts per kg of subject or sample weight. In certain aspects, a method for treating a condition described herein involves the administration to a subject in need thereof of a dosage of Compound 1 or a pharmaceutical composition thereof that ranges from about 0.001 mg/kg to about 500 mg/kg, from about 0.01 mg/kg to about 500 mg/kg, from about 0.1 mg/kg to about 500 mg/kg, from about 1 mg/kg to about 500 mg/kg, from about 10 mg/kg to about 500 mg/kg, from about 100 mg to about 500 mg/kg, from about 150 mg/kg to about 500 mg/kg, from about 250 mg/kg to about 500 mg/kg, or from about 300 mg/kg to about 500 mg/kg. In some aspects, a method for treating a condition described herein involves the administration to a subject in need thereof of a dosage of Compound 1 or a pharmaceutical composition thereof that ranges from about 0.001 mg/kg to about 100 mg/kg, from about 0.001 mg/kg to about 50 mg/kg, from about 0.001 mg/kg to about 25 mg/kg, from about 0.001 mg/kg to about 10 mg/kg, from about 0.001 mg/kg to about 5 mg/kg; from about 0.001 mg/kg to about 1 mg/kg; or from about 0.001 mg/kg to about 0.01 mg/kg. In accordance with these aspects, the dosage may be administered once, twice or three times per day, every other day, or once or twice per week and the dosage may be administered orally.

In certain aspects, a method for treating a condition described herein involves the administration to a subject in need thereof of a dosage of Compound 1 or a pharmaceutical composition thereof that ranges from about 0.01 mg/kg to about 100 mg/kg, from about 0.01 mg/kg to about 50 mg/kg, from about 0.01 mg/kg to about 25 mg/kg, from about 0.01 mg/kg to about 10 mg/kg, from about 0.01 mg/kg to about 5 mg/kg, from about 0.01 mg to about 1 mg/kg, or from about 0.01 mg/kg to about 0.1 mg/kg. In some aspects, a method for treating a condition described herein involves the administration to a subject in need thereof of a dosage of Compound 1 or a pharmaceutical composition thereof that ranges from about 0.1 mg/kg to about 100 mg/kg, from about 0.1 mg/kg to about 50 mg/kg, from about 0.1 mg/kg to about 25 mg/kg, from about 0.1 mg/kg to about 10 mg/kg, from about 0.1 mg/kg to about 5 mg/kg, from about 0.1 mg/kg to about 4 mg/kg; from about 0.1 mg/kg to about 3 mg/kg; from about 0.1 mg/kg to about 2 mg/kg; from about 0.1 mg to about 1.5 mg/kg, from about 0.1 mg to about 1.2 mg/kg, from about 0.1 mg to about 1 mg/kg, or from about 0.5 mg/kg to about 1.5 mg/kg. In accordance with these aspects, the dosage may be administered once, twice or three times per day, every other day, or once or twice per week and the dosage may be administered orally.

In specific aspects, a method for treating a condition described herein involves the oral administration to a subject in need thereof of a dosage of Compound 1 or a pharmaceutical composition thereof of about 0.1 mg/kg to about 5 mg/kg, about 0.1 mg/kg to about 4 mg/kg, about 0.1 mg/kg to about 3 mg/kg, about 0.1 mg/kg to about 2 mg/kg, about 0.5 mg/kg to about 2 mg/kg, or about 1 mg/kg to about 1.5 mg/kg is administered twice per day. In certain aspects, a method for treating a condition described herein involves the oral administration to a subject in need thereof of a dosage of Compound 1 or a pharmaceutical composition thereof of about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg or about 1 mg/kg twice per day. In certain specific aspects, a method for treating a condition described herein involves the oral administration to a subject in need thereof of a dosage of Compound 1 or a pharmaceutical composition thereof of about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, 1.9 mg/kg or about 2 mg/kg twice per day.

In specific aspects, a method for treating a condition described herein involves the administration to a subject in need thereof of Compound 1 or a pharmaceutical composition thereof at a dosage that achieves a target plasma concentration of the Compound in a subject with the neoplastic or the non-neoplastic condition or an animal model (e.g., an animal model with a pre-established human tumor). In a particular aspect, a method for treating a condition described herein involves the administration to a subject in need thereof of Compound 1 or a pharmaceutical composition thereof at a dosage that achieves a plasma concentration of the Compound ranging from approximately 0.001 μg/mL to approximately 100 mg/mL, approximately 0.01 μg/mL to approximately 100 mg/mL, approximately 0.01 μg/mL to approximately 10 mg/mL, approximately 0.1 μg/mL to approximately 10 mg/mL, approximately 0.1 μg/mL to approximately 500 μg/mL, approximately 0.1 μg/mL to approximately 200 μg/mL, approximately 0.1 μg/mL to approximately 100 μg/mL, or approximately 0.1 μg/mL to approximately 75 μg/mL in a subject with the neoplastic or the non-neoplastic condition or an animal model (e.g., an animal model with a pre-established human tumor). In specific aspects, a method for treating a condition described herein involves the administration to a subject in need thereof of Compound 1 or a pharmaceutical composition thereof at a dosage that achieves a plasma concentration of the Compound ranging from approximately 0.1 to approximately 50 μg/mL, approximately 0.1 μg/mL to approximately 25 μg/mL, approximately 0.1 μg/mL to approximately 20 μg/mL or approximately 5 μg/mL to approximately 10 μg/mL in a subject with the neoplastic or the non-neoplastic condition or an animal model (e.g., an animal model with a pre-established human tumor). To achieve such plasma concentrations, Compound 1 or a pharmaceutical composition thereof may be administered at doses that vary from 0.001 μg to 100,000 mg, depending upon the route of administration. In certain aspects, subsequent doses of Compound 1 may be adjusted accordingly based on the plasma concentrations of the Compound achieved with initial doses of the Compound or pharmaceutical composition thereof administered to the subject.

In specific aspects, a method for treating a condition described herein involves the administration to a subject in need thereof of Compound 1 or a pharmaceutical composition thereof at a dosage that achieves a target plasma concentration of DHODH in a subject with the neoplastic or the non-neoplastic condition or an animal model (e.g., an animal model with a pre-established human tumor). In a particular aspect, a method for treating a condition described herein involves the administration to a subject in need thereof of Compound 1 or a pharmaceutical composition thereof at a dosage that achieves a plasma concentration of DHODH ranging from approximately 0.1 pg/mL to approximately 100 mg/mL, approximately 0.1 pg/mL to approximately 1 mg/mL, approximately 0.1 pg/mL to approximately 500 μg/mL, approximately 0.1 pg/mL to approximately 500 μg/mL, approximately 0.1 pg/mL to approximately 100 μg/mL, or approximately 4 pg/mL to approximately 10 μg/mL in a subject with a condition described or an animal model (e.g., an animal model with a pre-established human tumor). To achieve such plasma concentrations, Compound 1 or a pharmaceutical composition thereof may be administered at doses that vary from 0.1 pg to 100,000 mg, depending upon the route of administration. In certain aspects, subsequent doses of Compound 1 or a pharmaceutical composition thereof may be adjusted accordingly based on the plasma concentrations of DHODH achieved with initial doses of the Compound or pharmaceutical composition thereof administered to the subject.

In particular aspects, a method for treating a condition described herein involves the administration to a subject in need thereof of Compound 1 or a pharmaceutical composition thereof at a dosage that achieves the desired tissue to plasma concentration ratios of the Compound as determined, e.g., by any imaging techniques known in the art such as whole-body autoradiography, in a subject with the neoplastic or the non-neoplastic condition or an animal model (such as an animal model with a pre-established human tumor).

In some aspects, a method for treating a condition described herein involves the administration to a subject in need thereof of one or more doses of an effective amount of Compound 1 or a pharmaceutical composition, wherein the effective amount may or may not be the same for each dose. In particular aspects, a first dose of Compound 1 or a pharmaceutical composition thereof is administered to a subject in need thereof for a first period of time, and subsequently, a second dose of Compound 1 is administered to the subject for a second period of time. The first dose may be more than the second dose, or the first dose may be less than the second dose. A third dose of Compound 1 also may be administered to a subject in need thereof for a third period of time.

In some aspects, the dosage amounts described herein refer to total amounts administered; that is, if more than one Compound is administered, then, in some aspects, the dosages correspond to the total amount administered. In a specific aspect, oral compositions contain about 5% to about 95% of a Compound by weight.

The length of time that a subject in need thereof is administered Compound 1 or a pharmaceutical composition in accordance with the methods for treating a condition described herein will be the time period that is determined to be efficacious. In certain aspects, a method for treating a condition described herein involves the administration of Compound 1 or a pharmaceutical composition thereof for a period of time until the severity and/or number of one or more symptoms associated with the neoplastic or the non-neoplastic condition decrease.

In some aspects, a method for treating a condition described herein involves the administration of Compound 1 or a pharmaceutical composition thereof for up to 48 weeks. In other aspects, a method for treating a condition described herein involves the administration of Compound 1 or a pharmaceutical composition thereof for up to 4 weeks, 8 weeks, 12 weeks, 16 weeks, 20 weeks, 24 weeks, 26 weeks (0.5 year), 52 weeks (1 year), 78 weeks (1.5 years), 104 weeks (2 years), or 130 weeks (2.5 years) or more. In certain aspects, a method for treating a condition described herein involves the administration of Compound 1 or a pharmaceutical composition thereof for an indefinite period of time. In some aspects, a method for treating a condition described herein involves the administration of Compound 1 or a pharmaceutical composition thereof for a period of time followed by a period of rest (i.e., a period wherein the Compound is not administered) before the administration of the Compound or pharmaceutical composition thereof is resumed. In specific aspects, a method for treating a condition described herein involves the administration of a Compound or pharmaceutical composition thereof in cycles, e.g., 1 week cycles, 2 week cycles, 3 week cycles, 4 week cycles, 5 week cycles, 6 week cycles, 8 week cycles, 9 week cycles, 10 week cycles, 11 week cycles, or 12 week cycles. In such cycles, Compound 1 or a pharmaceutical composition thereof may be administered once, twice, three times, or four times daily. In particular aspects, a method for treating a prostate condition presented herein involves the administration of Compound 1 or a pharmaceutical composition thereof twice daily in 4 week cycles.

In specific aspects, the period of time of administration of Compound 1 or pharmaceutical composition thereof may be dictated by one or more biomarker monitoring parameters, e.g., concentration of DHODH or other angiogenic or inflammatory mediators (e.g., cytokines or interleukins such as IL-6 or IL-8); tumor size, blood flow, or metabolism; peritumoral inflammation or edema. In particular aspects, the period of time of administration of Compound 1 or pharmaceutical composition thereof may be adjusted based on one or more monitoring parameters, e.g., concentration of DHODH or other angiogenic or inflammatory mediators (e.g., cytokines or interleukins such as IL-6 or IL-8); tumor size, blood flow, or metabolism; and/or peritumoral inflammation or edema.

In certain aspects, in accordance with the methods for treating a condition described herein, Compound 1 or a pharmaceutical composition thereof is administered to a subject in need thereof prior to, concurrently with, or after a meal (e.g., breakfast, lunch, or dinner). In specific aspects, in accordance with the methods for treating a condition described herein, Compound 1 or a pharmaceutical composition thereof is administered to a subject in need thereof in the morning (e.g., between 5 am and 12 pm). In certain aspects, in accordance with the methods for treating a condition described herein, Compound 1 or a pharmaceutical composition thereof is administered to a subject in need thereof at noon (i.e., 12 pm). In particular aspects, in accordance with the methods for treating a condition described herein, Compound 1 or a pharmaceutical composition thereof is administered to a subject in need thereof in the afternoon (e.g., between 12 pm and 5 pm), evening (e.g., between 5 pm and bedtime), and/or before bedtime.

In specific aspects, a dose of Compound 1 or a pharmaceutical composition thereof is administered to a subject once per day, twice per day, three times per day; once, twice or three times every other day (i.e., on alternate days); once, twice or three times every two days; once, twice or three times every three days; once, twice or three times every four days; once, twice or three times every five days; once, twice, or three times once a week, biweekly or monthly.

### Combination Therapy

Presented herein are combination therapies for the treatment of a condition described herein which involve the administration of Compound 1 in combination with one or more additional therapies to a subject in need thereof. In a specific aspect, presented herein are combination therapies for the treatment of a condition described herein which involve the administration of an effective amount of the Compound in combination with an effective amount of another therapy to a subject in need thereof.

As used herein, the term “in combination,” refers, in the context of the administration of a Compound, to the administration of a Compound prior to, concurrently with, or subsequent to the administration of one or more additional therapies (e.g., agents, surgery, or radiation) for use in treating a condition described herein. The use of the term “in combination” does not restrict the order in which one or more Compounds and one or more additional therapies are administered to a subject. In specific aspects, the interval of time between the administration of a Compound and the administration of one or more additional therapies may be about 1-5 minutes, 1-30 minutes, 30 minutes to 60 minutes, 1 hour, 1-2 hours, 2-6 hours, 2-12 hours, 12-24 hours, 1-2 days, 2 days, 3 days, 4 days, 5 days, 6 days, 7 days, 1 week, 2 weeks, 3 weeks, 4 weeks, 5 weeks, 6 weeks, 7 weeks, 8 weeks, 9 weeks, 10 weeks, 15 weeks, 20 weeks, 26 weeks, 52 weeks, 11-15 weeks, 15-20 weeks, 20-30 weeks, 30-40 weeks, 40-50 weeks, 1 month, 2 months, 3 months, 4 months 5 months, 6 months, 7 months, 8 months, 9 months, 10 months, 11 months, 12 months, 1 year, 2 years, or any period of time in between. In certain embodiments, a Compound and one or more additional therapies are administered less than 1 day, 1 week, 2 weeks, 3 weeks, 4 weeks, one month, 2 months, 3 months, 6 months, 1 year, 2 years, or 5 years apart.

In some aspects, the combination therapies provided herein involve administering Compound 1 daily, and administering one or more additional therapies once a week, once every 2 weeks, once every 3 weeks, once every 4 weeks, once every month, once every 2 months (e.g., approximately 8 weeks), once every 3 months (e.g., approximately 12 weeks), or once every 4 months (e.g., approximately 16 weeks). In certain aspects, Compound 1 and one or more additional therapies are cyclically administered to a subject. Cycling therapy involves the administration of the Compound for a period of time, followed by the administration of one or more additional therapies for a period of time, and repeating this sequential administration. In certain aspects, cycling therapy may also include a period of rest where the Compound or the additional therapy is not administered for a period of time (e.g., 2 days, 3 days, 4 days, 5 days, 6 days, 7 days, 1 week, 2 weeks, 3 weeks, 4 weeks, 5 weeks, 10 weeks, 20 weeks, 1 month, 2 months, 3 months, 4 months, 5 months, 6 months, 7 months, 8 months, 9 months, 10 months, 11 months, 12 months, 2 years, or 3 years). In one aspect, the number of cycles administered is from 1 to 12 cycles, from 2 to 10 cycles, or from 2 to 8 cycles.

In some aspects, the methods for treating a condition described herein comprise administering Compound 1 as a single agent for a period of time prior to administering the Compound in combination with an additional therapy. In certain aspects, the methods for treating a condition described herein comprise administering an additional therapy alone for a period of time prior to administering Compound 1 in combination with the additional therapy.

In some aspects, the administration of Compound 1 and one or more additional therapies in accordance with the methods presented herein have an additive effect relative the administration of the Compound or said one or more additional therapies alone. In some aspects, the administration of a Compound and one or more additional therapies in accordance with the methods presented herein have a synergistic effect relative to the administration of the Compound or said one or more additional therapies alone.

As used herein, the term “synergistic,” refers to the effect of the administration of a Compound in combination with one or more additional therapies (e.g., agents), which combination is more effective than the additive effects of any two or more single therapies (e.g., agents). In a specific aspect, a synergistic effect of a combination therapy permits the use of lower dosages (e.g., sub-optimal doses) of a Compound or an additional therapy and/or less frequent administration of a Compound or an additional therapy to a subject. In certain aspects, the ability to utilize lower dosages of a Compound or of an additional therapy and/or to administer a Compound or said additional therapy less frequently reduces the toxicity associated with the administration of a Compound or of said additional therapy, respectively, to a subject without reducing the efficacy of a Compound or of said additional therapy, respectively, in the treatment of a condition described herein. In some aspects, a synergistic effect results in improved efficacy of a Compound and each of said additional therapies in treating a condition described herein. In some aspects, a synergistic effect of a combination of a Compound and one or more additional therapies avoids or reduces adverse or unwanted side effects associated with the use of any single therapy.

The combination of Compound 1 and one or more additional therapies can be administered to a subject in the same pharmaceutical composition. Alternatively, the Compound and one or more additional therapies can be administered concurrently to a subject in separate pharmaceutical compositions. Compound 1 and one or more additional therapies can be administered sequentially to a subject in separate pharmaceutical compositions. Compound 1 and one or more additional therapies may also be administered to a subject by the same or different routes of administration.

The combination therapies provided herein involve administering to a subject to in need thereof Compound 1 in combination with conventional, or known, therapies for treating a condition described herein. Other therapies for a condition described herein or a condition associated therewith are aimed at controlling or relieving one or more symptoms. Accordingly, in some aspects, the combination therapies provided herein involve administering to a subject to in need thereof a pain reliever, or other therapies aimed at alleviating or controlling one or more symptoms associated with a condition described herein or a condition associated therewith.

Specific examples of anti-neoplastic agents that may be used in combination with Compound 1 include: a hormonal agent (e.g., aromatase inhibitor, selective estrogen receptor modulator (SERM), estrogen receptor antagonist or androgen antagonist), chemotherapeutic agent (e.g., microtubule dissembly blocker, antimetabolite, topisomerase inhibitor, and DNA crosslinker or damaging agent), anti-angiogenic agent (e.g., VEGF antagonist, receptor antagonist, integrin antagonist, vascular targeting agent (VTA)/vascular disrupting agent (VDA)), radiation therapy, and conventional surgery.

Non-limiting examples of hormonal agents that may be used in combination with Compound 1 include aromatase inhibitors, SERMs, and estrogen receptor antagonists. Hormonal agents that are aromatase inhibitors may be steroidal or nonsteroidal. Non-limiting examples of nonsteroidal hormonal agents include letrozole, anastrozole, aminoglutethimide, fadrozole, and vorozole. Non-limiting examples of steroidal hormonal agents include aromasin (exemestane), formestane, and testolactone. Non-limiting examples of hormonal agents that are SERMs include tamoxifen (branded/marketed as Nolvadex®), afimoxifene, arzoxifene, bazedoxifene, clomifene, femarelle, lasofoxifene, ormeloxifene, raloxifene, and toremifene. Non-limiting examples of hormonal agents that are estrogen receptor antagonists include fulvestrant. Other hormonal agents include but are not limited to abiraterone and lonaprisan.

Non-limiting examples of chemotherapeutic agents that may be used in combination with Compound 1 include microtubule disasssembly blocker, antimetabolite, topisomerase inhibitor, and DNA crosslinker or damaging agent. Chemotherapeutic agents that are microtubule dissemby blockers include, but are not limited to, taxenes (e.g., paclitaxel), docetaxel, abraxane, larotaxel, ortataxel, and tesetaxel); epothilones (e.g., ixabepilone); and vinca alkaloids (e.g., vinorelbine, vinblastine, vindesine, and vincristine).

Chemotherapeutic agents that are antimetabolites include, but are not limited to, folate anitmetabolites (e.g., methotrexate, aminopterin, pemetrexed, raltitrexed); purine antimetabolites (e.g., cladribine, clofarabine, fludarabine, mercaptopurine, pentostatin, thioguanine); pyrimidine antimetabolites (e.g., 5-fluorouracil, capcitabine, gemcitabine, cytarabine, decitabine, floxuridine, tegafur); and deoxyribonucleotide antimetabolites (e.g., hydroxyurea).

Chemotherapeutic agents that are topoisomerase inhibitors include, but are not limited to, class I (camptotheca) topoisomerase inhibitors (e.g., topotecan, irinotecan, rubitecan, and besampleecan); class II (podophyllum) topoisomerase inhibitors (e.g., etoposide or VP-16, and teniposide); anthracyclines (e.g., doxorubicin, epirubicin, Doxil, aclarubicin, amrubicin, daunorubicin, idarubicin, pirarubicin, valrubicin, and zorubicin); and anthracenediones (e.g., mitoxantrone and pixantrone).

Chemotherapeutic agents that are DNA crosslinkers (or DNA damaging agents) include, but are not limited to, alkylating agents (e.g., cyclophosphamide, mechlorethamine, ifosfamide, trofosfamide, chlorambucil, melphalan, prednimustine, bendamustine, uramustine, estramustine, carmustine, lomustine, semustine, fotemustine, nimustine, ranimustine, streptozocin, busulfan, mannosulfan, treosulfan, carboquone, N,N′N′-triethylenethiophosphoramide, triaziquone, triethylenemelamine); alkylating-like agents (e.g., carboplatin, cisplatin, oxaliplatin, nedaplatin, triplatin tetranitrate, satraplatin, picoplatin); nonclassical DNA crosslinkers (e.g., procarbazine, dacarbazine, temozolomide), altretamine, mitobronitol); and intercalating agents (e.g., actinomycin, bleomycin, mitomycin, and plicamycin).

Non-limiting examples of anti-angiogenic agents that may be used in combination with Compound 1 include VEGF antagonists, receptor antagonists, integrin antagonists (e.g., vitaxin, cilengitide, and S247), and VTAs/VDAs (e.g., fosbretabulin). VEGF antagonists include, but are not limited to, anti-VEGF antibodies (e.g., bevacizumab and ranibizumab), VEGF traps (e.g., aflibercept), VEGF antisense or siRNA or miRNA, and aptamers (e.g., pegaptanib). Anti-angiogenic agents that are receptor antagonists include, but are not limited to, antibodies (e.g., ramucirumab) and kinase inhibitors (e.g., sunitinib), sorafenib), cediranib, panzopanib, vandetanib, axitinib, and AG-013958) such as tyrosine kinase inhibitors. Other non-limiting examples of anti-angiogenic agents include ATN-224, anecortave acetate, microtubule depolymerization inhibitor such as combretastatin A4 prodrug, and protein or protein fragment such as collagen 18 (endostatin).

Non-limiting examples of other therapies that may be administered to a subject in combination with Compound 1 include:
(1) a statin such as lovostatin;
(2) an mTOR inhibitor such as sirolimus which is also known as Rapamycin, evorolimus, and deforolimus;
(3) a farnesyltransferase inhibitor agent such as tipifarnib;
(4) an antifibrotic agent such as pirfenidone;
(5) a pegylated interferon such as PEG-interferon alfa-2b;
(6) a CNS stimulant such as methylphenidate;
(7) a HER-2 antagonist such as anti-HER-2 antibody (e.g., trastuzumab) and kinase inhibitor (e.g., lapatinib);
(8) an IGF-1 antagonist such as an anti-IGF-1 antibody (e.g., AVE1642 and IMC-A11) or an IGF-1 kinase inhibitor;
(9) EGFR/HER-1 antagonist such as an anti-EGFR antibody (e.g., cetuximab, panitumamab) or EGFR kinase inhibitor (e.g., ersampleinib; gefitinib);
(10) SRC antagonist such as bosutinib or dasatinib;
(11) cyclin dependent kinase (CDK) inhibitor such as seliciclib;
(12) Janus kinase 2 inhibitor such as lestaurtinib;
(13) proteasome inhibitor such as bortezomib;
(14) phosphodiesterase inhibitor such as anagrelide;
(15) inosine monophosphate dehydrogenase inhibitor such as tiazofurine;
(16) lipoxygenase inhibitor such as masoprocol;
(17) endothelin antagonist;
(18) retinoid receptor antagonist such as tretinoin or alitretinoin;
(19) immune modulator such as lenalidomide, pomalidomide, or thalidomide;
(20) kinase (e.g., tyrosine kinase) inhibitor such as imatinib, dasatinib, ersampleinib, nisampleinib, gefitinib, sorafenib, sunitinib, lapatinib, or TG100801;
(21) non-steroidal anti-inflammatory agent such as celecoxib\;
(22) human granulocyte colony-stimulating factor (G-CSF) such as filgrastim;
(23) folinic acid or leucovorin calcium;
(24) integrin antagonist such as an integrin α5β-antagonist;
(25) nuclear factor kappa beta (NF-κβ) antagonist such as OT-551, which is also an anti-oxidant.
(26) hedgehog inhibitor such as CUR61414, cyclopamine, GDC-0449, and anti-hedgehog antibody;
(27) histone deacetylase (HDAC) inhibitor such as SAHA (also known as vorinostat), PCI-24781, SB939, CHR-3996, CRA-024781, ITF2357, JNJ-26481585, or PCI-24781;
(28) retinoid such as isotretinoin;
(29) hepatocyte growth factor/scatter factor (HGF/SF) antagonist such as HGF/SF monoclonal antibody;
(30) synthetic chemical such as antineoplaston;
(31) anti-diabetic such as rosaiglitazone;
(32) antimalarial and amebicidal drug such as chloroquine;
(33) synthetic bradykinin such as RMP-7;
(34) platelet-derived growth factor receptor inhibitor such as SU-101;
(35) receptor tyrosine kinase inhibitors of Flk-1/KDR/VEGFR2, FGFR1 and PDGFR beta such as SU5416 and SU6668;
(36) anti-inflammatory agent such as sulfasalazine;
(37) IL-6 pathway inhibitors such as tocilizumab; and
(38) TGF-beta antisense therapy.

Non-limiting examples of other therapies that may be administered to a subject in combination with Compound 1 include: a synthetic nonapeptide analog of naturally occurring gonadotropin releasing hormone such as leuprolide acetate; a nonsteroidal, anti-androgen such as flutamide or nilutamide; a non-steroidal androgen receptor inhibitor such as; steroid hormone such as progesterone; anti-fungal agent such as glucocorticoid such as prednisone; estramustine phosphate sodium; and bisphosphonate such as pamidronate, alendronate, and risedronate.

Other specific examples of therapies that may be used in combination with Compound 1 include, but are not limited to, antibodies that specifically bind to a tumor specific antigen or tumor associated antigen, e.g., anti-EGFR/HER-1 antibodies.

Additional specific examples of therapies that may be used in combination with Compound 1 include, but are not limited to, agents associated with immunotherapy, e.g., cytokines, interleukins, and vaccines.

Specific examples of agents alleviating side-effects associated with a condition described herein that can be used as therapies in combination with Compound 1, include, but are not limited to: antiemetics, e.g., Ondansetron hydrochloride, Granisetron hydrochloride, Lorazepam and Dexamethasone.

In certain aspects, combination therapies provided herein for treating a condition described herein comprise administering Compound 1 in combination with one or more agents used to treat and/or manage a side effect, such as, bleeding (usually transient, low-grade epistaxis), arterial and venous thrombosis, hypertension, delayed wound healing, asymptomatic proteinuria, nasal septal perforation, reversible posterior leukoencephalopathy syndrome in association with hypertension, light-headedness, ataxia, headache, hoarseness, nausea, vomiting, diarrhea, rash, subungual hemorrhage, myelosuppression, fatigue, hypothyroidism, QT interval prolongation, or heart failure.

In certain embodiments, Compound 1 is not used in combination with a drug that is primarily metabolized by CYP2D6 (such as an antidepressant (e.g, a atricyclic antidepressant, a selective serotonin reuptake inhibitor, and the like), an antipsychotic, a beta-adrenergic receptor blocker, or certain types of anti-arrhythmics) to treat a condition described herein.

### Kits

Provided herein is a pharmaceutical pack or kit comprising one or more containers filled with Compound 1 or a pharmaceutical composition thereof. Additionally, one or more other therapies useful for the treatment of a condition, or other relevant agents can also be included in the pharmaceutical pack or kit. Also provided herein is a pharmaceutical pack or kit comprising one or more containers filled with one or more of the ingredients of the pharmaceutical compositions described herein. Optionally associated with such kits can be a notice in the form prescribed by a governmental agency regulating the manufacture, use or sale of pharmaceuticals or biological products, which notice reflects approval by the agency of manufacture, use or sale for human administration.

### Patient Population

In some embodiments, a subject treated for a condition described herein in accordance with the methods provided herein is a human who has or is diagnosed with a condition described herein. In other aspects, a subject treated for a condition described herein in accordance with the methods provided herein is a human predisposed or susceptible to a condition described herein. In some aspects, a subject treated for a condition described herein in accordance with the methods provided herein is a human at risk of developing a condition described herein.

In one aspect, a subject treated for a condition described herein in accordance with the methods provided herein is a human infant. In another aspect, a subject treated for a condition described herein in accordance with the methods provided herein is a human toddler. In another aspect, a subject treated for a condition described herein in accordance with the methods provided herein is a human child. In another aspect, a subject treated for a condition described herein in accordance with the methods provided herein is a human adult. In another aspect, a subject treated for a condition described herein in accordance with the methods provided herein is a middle-aged human. In another aspect, a subject treated for a condition described herein in accordance with the methods provided herein is an elderly human.

In certain aspects, a subject treated for a neoplasm in accordance with the methods provided herein has a malignant neoplasm that metastasized to other areas of the body, such as the bones, lung and liver. In certain aspects, a subject treated for a neoplasm in accordance with the methods provided herein has a neoplasm that is in remission. In some aspects, a subject treated for a neoplasm in accordance with the methods provided herein that has a recurrence of the neoplastic condition. In certain aspects, a subject treated in accordance with the methods provided herein is experiencing recurrence of one or more tumors associated with a neoplasm.

In certain aspects, a subject treated for a neoplasm or a non-neoplastic condition in accordance with the methods provided herein is a human that is about 1 to about 5 years old, about 5 to 10 years old, about 10 to about 18 years old, about 18 to about 30 years old, about 25 to about 35 years old, about 35 to about 45 years old, about 40 to about 55 years old, about 50 to about 65 years old, about 60 to about 75 years old, about 70 to about 85 years old, about 80 to about 90 years old, about 90 to about 95 years old or about 95 to about 100 years old, or any age in between. In a specific aspect, a subject treated for a neoplasm or a non-neoplastic condition in accordance with the methods provided herein is a human that is 18 years old or older. In a particular aspect, a subject treated for a neoplasm or a non-neoplastic condition in accordance with the methods provided herein is a human child that is between the age of 1 year old to 18 years old. In a certain aspect, a subject treated for a neoplasm or a non-neoplastic condition in accordance with the methods provided herein is a human that is between the age of 12 years old and 18 years old. In a certain aspect, the subject is a male human. In another aspect, the subject is a female human. In one aspect, the subject is a female human that is not pregnant or is not breastfeeding. In one aspect, the subject is a female that is pregnant or will/might become pregnant, or is breast feeding.

In particular aspects, a subject treated for a neoplasm or a non-neoplastic condition in accordance with the methods provided herein is a human that is in an immunocompromised state or immunosuppressed state. In certain aspects, a subject treated for a neoplasm or a non-neoplastic condition in accordance with the methods provided herein is a human receiving or recovering from immunosuppressive therapy. In certain aspects, a subject treated for a neoplasm or a non-neoplastic condition in accordance with the methods provided herein is a human that has or is at risk of getting a malignant neoplasm (e.g., metastatic cancer), AIDS, or a bacterial infection. In certain aspects, a subject treated for a neoplasm or a non-neoplastic condition in accordance with the methods provided herein is a human who is, will or has undergone surgery, drug therapy, such as chemotherapy, hormonal therapy and/or radiation therapy.

In specific aspects, a subject treated for a neoplasm or a non-neoplastic condition in accordance with the methods provided herein is suffering from a condition, e.g., stroke or cardiovascular conditions that may require VEGF therapy, wherein the administration of anti-angiogenic therapies other than a Compound may be contraindicated. For example, in certain aspects, a subject treated for a neoplasm or a non-neoplastic condition in accordance with the methods provided herein has suffered from a stroke or is suffering from a cardiovascular condition. In some aspects, a subject treated for a neoplasm or a non-neoplastic condition in accordance with the methods provided herein is a human experiencing circulatory problems. In certain aspects, a subject treated for a neoplasm or a non-neoplastic condition in accordance with the methods provided herein is a human with diabetic polyneuropathy or diabetic neuropathy. In some aspects, a subject treated for a neoplasm or a non-neoplastic condition in accordance with the methods provided herein is a human receiving VEGF protein therapy. In other aspects, a subject treated for a neoplasm or a non-neoplastic condition in accordance with the methods provided herein is not a human receiving VEGF protein therapy.

In some aspects, a subject treated for a neoplasm or a non-neoplastic condition in accordance with the methods provided herein is administered a Compound or a pharmaceutical composition thereof, or a combination therapy before any adverse effects or intolerance to therapies other than the Compound develops. In some aspects, a subject treated for a neoplasm or a non-neoplastic condition in accordance with the methods provided herein is a refractory patient. In a certain aspects, a refractory patient is a patient refractory to a standard therapy (e.g., surgery, radiation, anti-androgen therapy and/or drug therapy such as chemotherapy). In certain aspects, a patient with a neoplasm or a non-neoplastic condition is refractory to a therapy when the neoplasm or the non-neoplastic condition has not significantly been eradicated and/or the one or more symptoms have not been significantly alleviated. The determination of whether a patient is refractory can be made either in vivo or in vitro by any method known in the art for assaying the effectiveness of a treatment of a neoplasm or a non-neoplastic condition, using art-accepted meanings of “refractory” in such a context. In various aspects, a patient with a neoplasm is refractory when one or more tumors associated with the neoplasm, have not decreased or have increased. In various aspects, a patient with a neoplasm is refractory when one or more tumors metastasize and/or spread to another organ.

In some aspects, a subject treated for a neoplasm or a non-neoplastic condition in accordance with the methods provided herein is a human that has proven refractory to therapies other than treatment with a Compound, but is no longer on these therapies. In certain aspects, a subject treated for a neoplasm or a non-neoplastic condition in accordance with the methods provided herein is a human already receiving one or more conventional anti-neoplastic therapies, such as surgery, drug therapy such as chemotherapy, anti-androgen therapy or radiation. Among these patients are refractory patients, patients who are too young for conventional therapies, and patients with recurring tumors despite treatment with existing therapies.

In some aspects, a subject treated for a neoplasm or a non-neoplastic condition in accordance with the methods provided herein is a human susceptible to adverse reactions to conventional therapies. In some aspects, a subject treated for a neoplasm or a non-neoplastic condition in accordance with the methods provided herein is a human that has not received a therapy, e.g., drug therapy such as chemotherapy, surgery, anti-androgen therapy or radiation therapy, prior to the administration of Compound 1 or a pharmaceutical composition thereof. In other aspects, a subject treated for a neoplasm or a non-neoplastic condition in accordance with the methods provided herein is a human that has received a therapy prior to administration of Compound 1. In some aspects, a subject treated for a neoplasm or a non-neoplastic condition in accordance with the methods provided herein is a human that has experienced adverse side effects to the prior therapy or the prior therapy was discontinued due to unacceptable levels of toxicity to the human.

In some aspects, a subject treated for a neoplasm or a non-neoplastic condition in accordance with the methods provided herein has had no prior exposure to another anti-angiogenic therapy (e.g., an anti-VEGF monoclonal antibody, an anti-VEGFR monoclonal antibody, a tyrosine kinase inhibitor, or other angiogenesis pathway modulator). In particular aspects, a subject treated for a neoplasm or a non-neoplastic condition in accordance with the methods provided herein does not have uncontrolled hypertension, major bleeding, HIV infection or recent acute cardiovascular event. In some aspects, a subject treated for a neoplasm or a non-neoplastic condition in accordance with the methods provided herein has myocardial infarction, unstable angina, coronary/peripheral artery bypass graft, congestive heart failure, cerebrovascular accident, transient ischemic attack, an arterial thromboembolic event, or pulmonary embolism.

In some aspects, a subject treated for a neoplasm or a non-neoplastic condition in accordance with the methods provided herein is not, has not and/or will not receive a drug that is primarily metabolized by CYP2D6. In particular aspects, a subject treated for a neoplasm or a non-neoplastic condition in accordance with the methods provided herein has not and will not received a drug that is primarily metabolized by CYP2D6 1, 2, 3 or 4 weeks before receiving a Compound or a pharmaceutical composition thereof and 1, 2, 3 or 4 weeks after receiving the Compound or pharmaceutical composition. Examples of such drugs include, without limitation, some antidepressants (e.g., tricyclic antidepressants and selective serotonin uptake inhibitors), some antipsychotics, some beta-adrenergic receptor blockers, and certain anti-arrhythmics. In specific aspects, a subject treated for a neoplasm or a non-neoplastic condition in accordance with the methods provided herein is not, has not and/or will not receive tamoxifen. In particular aspects, a subject treated for a neoplasm or a non-neoplastic condition in accordance with the methods provided herein has not and will not received tamoxifen 1, 2, 3 or 4 weeks before receiving a Compound or a pharmaceutical composition thereof and 1, 2, 3 or 4 weeks after receiving the Compound or pharmaceutical composition. In specific aspects, a subject treated for a neoplasm or a non-neoplastic condition in accordance with the methods provided herein has received tamoxifen, e.g., for 1, 2, 3 or 4 weeks before receiving a Compound or a pharmaceutical composition thereof.

### SPECIFIC EXAMPLES

The present invention will be further understood by reference to the following non-limiting, specific examples. In particular, the examples demonstrate the use of capsule and tablet dosage forms and the effect of excipient selection on Compound (Cpd) 1 loading, solubility and bioavailability in a fed or fasted state and the stability of SDIs comprising Compound 1 and a polymer in various compositions and formulations.

### Example 1

### Spray Dried Intermediate Solubility Studies

### Materials & Methods

The following excipients and other materials were used (Table 2) in the preparation of formulations described herein.

TABLE 2
Active and Inactive Materials
Material
Compound 1a crystalline
Compound 1b crystalline
Polyvinylpyrrolidone K-30 polymer (PVP K30)
Polyvinylpyrrolidone K-90 polymer (PVP K90)
Methocel E5 (HPMC low viscosity grade)
Sodium dodecyl sulfate (SDS or SLS)
Poloxamer 188 (Pol 188)
Poloxamer 407 (Pol 407)
Gelucire 44/14 (Gel 44/14)
Gelucire 50/13 (Gel 50/13)
Microcrystalline cellulose Avicel-102 (MCC)
Croscarmellose Sodium Type A (CCS)

### Process for Preparing a Spray Dried Intermediate

For use in the described compositions and formulations, a solution of the crystalline form of Compound 1a or Compound 1b and optional excipients were co-precipitated by spray-drying using a Mini Büchi B-290 laboratory scale spray-dryer. The solutions were sprayed through a nozzle by a peristaltic pump to provide a SDI comprising Compound 1 as an amorphous form and optional co-precipitated excipients. The obtained SDI samples were kept under vacuum for a period of 24 hrs at RT in order to remove residual solvent.

The term “composition” refers to a product comprising an SDI as described herein and optional excipients prepared in solution using techniques known to those skilled in the art.

The term “formulation” refers to a product comprising a composition as described herein and additional excipients prepared using dry blending techniques known to those skilled in the art.

X-Ray Powder Diffraction (XRPD): The crystalline or polymorph character of the SDI was determined by XRPD using a Siemens D-5000 X-ray diffractometer with Co αK radiation (λ=1.7890 Å) at a scanning speed of 0.017°2θs⁻¹ over a range of 3-40°2θ.

Differential Scanning Calorimetry (DSC): DSC measurements were obtained using a Mettler Toledo Differential Scanning Calorimeter model DSC1 at a temperature range from 25 to 240° C. and heating rate of 10 and 25° C. min⁻¹ under a nitrogen purge of 50 mL min⁻¹. Indium was used as calibration standard. The sample was analyzed using a sealed aluminum pan (40 μL).

Thermogravimetric analysis (TGA): The TGA analysis was performed by an automated modular Mettler Toledo TGA/DSC1. The analysis was done in a controlled atmosphere of nitrogen purged at 50 mL min⁻¹. The temperature range was from 25 to 120° C. at a heating rate of 10° C. min⁻¹. For TGA, the samples were placed in an aluminum-oxide crucible (70 μL).

Water determination: Water determination was performed by Karl Fisher Titration using a Metrohm Titrino Model 795. A commercially prepared reagent titer containing imidazole, iodine, sulfur dioxide, and ether in proportions such that 1 mL of reagent would react to approximately 2 mg of H₂O. The system was equipped with suitable desiccants and the solution was calibrated before each series of sample analysis performing 3 measurements using exactly 10 μL of purified water. The relative standard deviation of these measurements was limited to less than or equal to 2.0%.

### Polymer Selection and Compound Loading

SDI batches (Table 3) were prepared using the process described above with Compound 1 loading in a range of from 40% up to 70%. For SDI batches made with PVP K30 the highest loading of Compound 1 was 80%. A loading in a range of from 50% up to 70% was selected to evaluate PVP K90 and HPMC E5 as polymers in the SDIs. Each SDI batch was obtained by dissolving Compound 1 and the polymer in a DCM:EtOH 80:20 solvent system (200 mL) under stirring at room temperature (RT).

TABLE 3
Polymer Selection and Loading

| Sample | | | | | |
|---|---|---|---|---|---|
| No. | Load (%) | (gm) | Polymer | (%) | (gm) |
| 1 | 40 | 2.0 | PVP K30 | 60 | 3.0 |
| 2 | 100 | 2.5 | — | 0 | 0 |
| 3 | 50 | 2.5 | PVP K30 | 50 | 2.5 |
| 4 | 60 | 3.0 | PVP K30 | 40 | 2.0 |
| 5 | 70 | 3.5 | PVP K30 | 30 | 1.5 |
| 6 | 80 | 4.0 | PVP K30 | 20 | 1.0 |
| 7 | 50 | 2.5 | PVP K90 | 50 | 2.5 |
| 8 | 70 | 3.5 | PVP K90 | 30 | 1.5 |
| 9 | 50 | 2.5 | HPMC E5 | 50 | 2.5 |
| 10 | 70 | 3.5 | HPMC E5 | 30 | 1.5 |

As described herein, SDI samples were incubated under accelerated conditions 40° C./75% relative humidity in open and polypropylene (PP) capped high density polyethylene (HDPE) bottles to determine amorphous form stability.

The samples were evaluated by:
1—XRPD for amorphous state at time zero and at subsequent time points on stability station
2—Kinetic solubility for up to 24 hours in aqueous solutions (5% w/w) of SDS, Poloxamer 188, Poloxamer 407, Gelucire 44/14 and Gelucire 50/13 at time zero
3—DSC and TGA thermograms at time zero and DSC at subsequent time points on stability station
4—Solubility at an in vivo representative concentration of 0.4 mg/mL of Compound 1 in water and in aqueous solutions (5% w/w) of SDS, Poloxamer 188, Poloxamer 407, Gelucire 44/14 and Gelucire 50/13 at time zero.

### Spray-Drying Process and Results

To obtain a stable SDI with improved solubility and to avoid in vivo precipitation or recrystallization of Compound 1, the spray-drying technique described above was used to obtain a SDI comprising a precipitated amorphous Compound 1 complexed with a polymer according to Table 3.

In order to produce the SDIs, the crystalline Compound 1a or Compound 1b was complexed with either PVP-K30 and HPMC using the spray-drying process parameters shown in Table 4. The composition of each SDI, as well as the obtained yield, are shown in Table 5.

As shown in Table 4, at greater concentrations of Compound 1a or Compound 1 b, and/or when PVP90 was used, the process parameters were modified in order to maximize the yield. In general, the selected polymers were easily dissolved into the solvent system, resulting in non-viscous clear/transparent liquids, with the exception of SDI Sample 9 (HPMC 50%) wherein some very fine particles remained in suspension.

In Table 4, the inlet temperature (In) and outlet temperature (Out) are shown in ° C., the atomization air pressure (Air) is shown in mm, the spray gas flow (Gas) is shown in L/h, the aspirator rate (Asp) and pump rate (Pmp) are shown in %, the volume flow (Vol) is shown in m³/h and the feed flow (Feed) is shown in mL/min. The spray gas flow was determined according to manufacturer's specifications and the actual feed flow was calculated by the volume of solution used according to pump run time.

The term “Yieldₜₒₜ” refers to the percentage (%) of the total yield calculated by combining the material recuperated from the spraying cylinder, the cyclone and the collection vessel. The term “Yield_{ccv}” refers to the percentage (%) of the combined amounts recovered from the cyclone/collection vessel.

TABLE 4
Spray-Drying Process Parameters

| Sample | | | | | | | | |
|---|---|---|---|---|---|---|---|---|
| No. | In | Out | Air | Gas | Asp | Vol | Pmp | Feed |
| 2 | 65-67 | 36-42 | 40 | 473 | 90 | 35 | 25 | 7.5 |
| 3 | 64-67 | 41-45 | 40 | 473 | 90 | 35 | 20 | 6.0 |
| 4 | 63-66 | 41-46 | 40 | 473 | 90 | 35 | 20 | 6.0 |
| 5 | 63-66 | 42-45 | 40 | 473 | 90 | 35 | 20 | 6.0 |
| 6 | 63-67 | 43-49 | 40 | 473 | 90-95 | 35-37 | 20-25 | 6.0-7.5 |
| 7 | 74-77 | 43-46 | 25-40 | 301-473 | 90-95 | 35-37 | 10-20 | 3.5-6.0 |
| 8 | 63-71 | 47-38 | 30-50 | 357-536 | 90 | 35 | 10-20 | 3.5-6.0 |
| 9 | 62-69 | 41-45 | 40 | 473 | 90 | 35 | 20 | 6.0 |
| 10 | 64-67 | 40-43 | 40 | 473 | 90 | 35 | 20 | 6.0 |

TABLE 5
SDI Yield

| Sample No. | Yieldₜₒₜ(%) | Yield_{ccv} (%) |
|---|---|---|
| 2 | 67.1 | ND |
| 3 | 44.5 | 44.5 |
| 4 | 50.8 | 50.8 |
| 5 | 42.8 | 30.7 |
| 6 | 49.8 | 26.8 |
| 7 | 35.1 | 0.0 |
| 8 | 70.3 | 30.8 |
| 9 | 76.0 | 76.0 |
| 10 | 76.1 | 76.1 |

In general, precipitated SDI Samples had the desired particle size, but the particles had a sticky nature.

The SDI Samples from Samples 9 and 10 were retrieved solely from the collection vessel.

The SDI Samples from Samples 7 and 8 provided fine sticky cohesive agglomerates with relatively poor flowability. In addition, these Samples formed fibers during spray-drying. Concentrations of PVP K90 (Sample 7) at 50% resulted in complete filament formation in the drying chamber. Varying the process parameters within the ranges shown in Table 5 did not avoid formation of the fibers. Fiber formation appeared to be a function of the PVP K90 concentration. A reduction to 30% PVP K90 (Sample h) provided a relatively low yield (30.8%) in the collection vessel.

### SDI Analytical Results

### XRPD

The amorphous content of the co-precipitated SDI batches was evaluated by XRPD. The XRPD pattern of each SDI sample was typical of an amorphous material.

### TGA

TGA measurements showed the rate of mass loss as a function of sample temperature and time, with no significant mass loss between 25 and 120° C. Residual solvents were not detected in the tested product. The volatilization of residual solvent is typically associated with the initial weight loss of the sample during TGA.

### DSC

DSC thermograms were obtained at a heating rate of 10° C./min. Each SDI showed endothermic peak (Endo Peak) inflections between 85° C. and 105° C. at the glass transition temperature (GTT) (in ° C.) and heat capacity (HC) (in J g⁻¹ k⁻¹) are shown in Table 6. In general, the results show that the glass transition temperature is a function of the amount and type of polymer used in the composition.

Endothermic peaks (Endo) (in ° C.) may be associated to the occurrence of various crystal modifications with different melting points. Exothermic peaks (Exo) (in ° C.) could be explained by crystallization, solid-solid transitions, decomposition or chemical reactions. The enthalpy for each peak (Ey) (in J g⁻¹) is also shown in Table 6.

For Sample 3, no detectable exothermic peak transition was seen. For Samples 5 and 6, exothermic transitions usually associated with material crystallization were seen. For Sample 6, an endothermic transition representing the melting process occurred between 158° C. and 224° C. The areas of the exothermic transition peaks were directly proportional to the SDI Compound 1 concentration.

For Samples 8, 9, and 10 no detectable exothermic peak was seen.

For Samples 4 and 5, a second small endothermic peak having a melting temperature of 207 and 221° C., respectively, was observed.

For Samples 3 and 6, comparative DSC thermograms obtained at a heating rate of 25° C./min generally showed an increase in the area of the peaks observed. For Sample 6, the 25° C./min thermogram was similar to the 10° C./min thermogram, with a slightly higher shift in the exothermic peak temperature at 25° C./min.

The term “NO” represents “Not Observed” and the term “ND” represents “Not Determined.”

TABLE 6
DSC Results

| Sample No. | GTT | HC | Exo | Ey | Endo | Ey |
|---|---|---|---|---|---|---|
| 1 | 102.2 | 0.57 | NO | NO | 187.3 | −40.65 |
| 2 | 86.7 | ND | 133.75 | 65.35 | 224.0 | −105.10 |
| 3 | 96.4 | 1.06 | NO | ND | NO | ND |
| 4 | 95.4 | 0.66 | NO | ND | 188.8/206.5 | −16.17/−1.75 |
| 5 | 95.9 | 0.52 | 176.0 | 14.39 | 210.9 | −33.23 |
| 6 | 91.5 | 0.50 | 165.9 | 48.77 | 217.4 | −55.30 |
| 7 | 92.9 | 0.55 | NO | ND | 173.7 | −25.57 |
| 8 | 102.5 | 0.39 | 185.3 | 9.79 | 212.4 | −22.03 |
| 9 | 93.5 | 0.62 | NO | ND | 189.5 | −2.91 |
| 10 | 89.1 | 0.47 | NO | ND | 158.7/221.4 | −2.63/−0.50 |

Solubility of the SDI and a Surfactant (5% w/w) in Aqueous Solution

The solubility of the crystalline Compound 1b and the amorphous SDI was measured in different aqueous media at RT with sampling times at 0 and 24 hrs (Tables 7 and 8). Aqueous suspensions (5% w/w) of the crystalline Compound 1b and the amorphous SDI were prepared in saturated solutions with surfactants selected from SDS, Poloxamer 188, Poloxamer 407, Gelucire 44/14 and Gelucire 50/13.

The Gelucire 50/13 solution was opaque, indicating that not all the Gelucire was solubilized. The solution was not sufficiently translucent to determine when sufficient material was added to provide the saturated solution. As a result, the solution was allowed to stand so that the excess Gelucire settled, and the supernatant was recovered and used for the solubility study. The exact concentration of Gelucire 50/13 in the solution was unknown. Solutions of the SDI in Gelucire 50/13 showed considerable precipitation after 24 hrs.

The term “NR” represents “Not Reproducible,” and the term “SP” represents “Significant Precipitation.”

TABLE 7
Solubility (μg/mL)

| | | Compound | | |
|---|---|---|---|---|
| Solution | Time | 1b | Sample 2 | Sample 1 |
| SDS | 0 hrs | 2.4 | 224.7 | 1756 |
| | 24 hrs | 6.2 | 32.4 | 195.4 |
| Pol 188 | 0 hrs | 0.2 | 0.7 | 2.8 |
| | 24 hrs | 0.0 | 4.3 | 2.2 |
| Gel 44/14 | 0 hrs | 14.7 | 142.5 | 124.7 |
| | 24 hrs | 23.6 | 58.4 | 57.6 |
| Gel 50/13 | 0 hrs | 7.8 | 677.4 | 2939 |
| | 24 hrs | 23.0 | 371.5 | 128.1 |

TABLE 8
Solubility (μg/mL)

| Solu- | | | | | | | | | |
|---|---|---|---|---|---|---|---|---|---|
| tions | Time | 3 | 4 | 5 | 6 | 7 | 8 | 9 | 10 |
| SDS | 0 hrs | 0.0 | 152.9 | 0.0 | 201.6 | 0.0 | 94.9 | 134.6 | 180.8 |
| | 24 hrs | SP | SP | SP | 154.6 | SP | SP | SP | SP |
| Pol | 0 hrs | 3.1 | 0.5 | 164.2 | 2.9 | 6.2 | 0.0 | 0.9 | 716.9 |
| 188 | 24 hrs | SP | SP | SP | SP | SP | SP | SP | SP |
| Pol | 0 hrs | 0.0 | 0.0 | 7.7 | 101.0 | 739. | 4 0.0 | NR | 0.0 |
| 407 | 24 hrs | SP | SP | SP | SP | SP | SP | SP | SP |
| Gel | 0 hrs | 52.5 | 73.0 | 105.2 | 66.9 | 58.1 | 69.1 | 130.3 | 0.0 |
| 44/14 | 24 hrs | SP | SP | SP | SP | SP | SP | SP | SP |
| Gel | 0 hrs | 133.2 | 560.0 | 0.0 | 280.8 | 156.8 | 0.0 | 0.0 | 1579 |
| 50/13 | 24 hrs | 102.3 | 310.2 | SP | 132.1 | SP | SP | SP | 1421 |

The solubility of Samples c and e in SDS was verified (Table 9). The SDS solution was clear and allowed the SDI to be added to the vessel in excess. The solution was sonicated for 5 minutes, shaken for 2 minutes, and left to rest for 5 minutes. The supernatant was transferred to an HPLC vial through a syringe with a 0.45 um nylon filter. The samples were injected immediately (the system had been pre-conditioned before starting the solubility test).

TABLE 9
Solubility (μg/mL)

| | | Sample | Sample |
|---|---|---|---|
| Solution | Time | c | e |
| SDS | 0 hrs | 12116* | 1809 |
| | 2 hrs | 248.9 | 507.9 |
| | 24 hrs | 0.0 | 163.8 |

*This value could not be reproduced.

The results shown in Tables 8 and 9 indicate that the choice of PVP and HPMC polymers in the SDI influenced solubility in the presence of a surfactant.

Solubility of SDI (300-400 μg/mL) and a Surfactant (5% w/w) in Aqueous Solution

Depending on the desired dose loading, various amounts of SDI were dissolved in saturated solutions with surfactants selected from SDS, Poloxamer 188, Poloxamer 407, Gelucire 44/14 and Gelucire 50/13 at 37° C. (as shown in Table 10).

For example, an amount of SDI between 75% (0.3 mg/mL) and 95% (0.4 mg/mL) was dissolved in a solution with Poloxamer 407 or Gelucire 50/13. A relatively lesser amount of SDI was capable of being dissolved into the SDS, Poloxamer 188 and Gelucire 44/14 solutions. At the nominal SDI concentration of 0.4 mg/mL, the SDS, Poloxamer 407 and Gelucire 50/13 solutions kept the SDI in solution for up to 2 hours.

For PVP K30 Sample 5 and PVP K90 Sample 8, greater solubility was achieved at a 70% Compound 1 loading. Samples 5 and 8 were able to maintain the SDI concentrations in Gelucire 50/13 and Poloxamer 407 and to a certain extent in SDS.

For HPMC E5 Samples 9 and 10, an SDI concentration of about 0.2 to about 0.3 mg/mL was maintained in all solutions except Poloxamer 188. For Samples 9 and 10, there was no correlation between Compound 1 loading and solubility.

TABLE 10
Solubility (μg/mL) at 400 μg/mL

| | Time | 1 | 2 | 3 | 4 | 5 | 6 | 8 | 9 | 103 |
|---|---|---|---|---|---|---|---|---|---|---|
| Water | 0 hrs | 0.0 | 0.0 | 0.0 | 0.0 | 0.0 | 0.0 | 0.0 | 0.0 | 0.0 |
| | 2 hrs | 0.0 | 0.0 | 0.0 | 0.0 | 0.0 | 0.8 | 0.0 | 0.0 | 0.0 |
| SDS | 0 hrs | 69.0 | 387.3 | 213.5 | 295.6 | 197.4 | 93.2 | 74.9 | 302.3 | 247.7 |
| | 2 hrs | 54.3 | 97.0 | 92.6 | 235.6 | 87.6 | 38.8 | 54.5 | 274.3 | 269.0 |
| Pol 188 | 0 hrs | 80.9 | 56.3 | 92.8 | 18.0 | 105.1 | 19.7 | 175.2 | 276.0 | 129.6 |
| | 2 hrs | 0.1 | 0.1 | 0.6 | 0.6 | 0.0 | 0.5 | 0.1 | 24.3 | 0.9 |
| Pol 407 | 0 hrs | 133.3 | 300.2 | 287.4 | 360.0 | 326.6 | 162.2 | 346.0 | 319.5 | 263.4 |
| | 2 hrs | 138.8 | 310.1 | 313.2 | 357.3 | 364.3 | 151.0 | 369.7 | 371.2 | 363.2 |
| Gel 44/14 | 0 hrs | 85.0 | 291.8 | 380.2 | 336.1 | 98.9 | 222.5 | 380.6 | 331.7 | 333.2 |
| | 2 hrs | 87.4 | 43.9 | 0.0 | 153.1 | 101.7 | 67.5 | 169.1 | 116.5 | 318.2 |
| Gel 50/13 | 0 hrs | 156.9 | 332.3 | 264.3 | 331.8 | 282.8 | 312.7 | 349.7 | 261.7 | 184.5 |
| | 2 hrs | 155.5 | 239.6 | 299.7 | 336.6 | 323.1 | 264.6 | 362.0 | 327.8 | 352.4 |

### Example 2

### Solubility of Selected SDI-Surfactant Compositions

Compositions having a maximum Compound 1 loading of 50-60% showed limited crystallization of the amorphous form at high temperature according to DSC and hot stage microscopy data.

Short term stability studies indicated no significant differences between use of either PVP K30 or HPMC E5 with regard to Compound 1 crystallization/recrystallization. Both showed favorable solubility characteristics for the SDI. However, dissolution comparisons between the PVP SDI and HPMC E5 SDI showed a much improved dissolution profile for the HPMC E5 SDI.

### HPMC E5 Solubility

HPMC E5, HPMC E5/Cpd 1 and HPMC E5/Cpd 1/Poloxamer 407 solubility was tested in various organic solvents (see results shown in Tables 11 and 12). The compositions were dispersed individually into the solvent under stirring at RT. HPMC E5 and Poloxamer 407 (when used) were dissolved first to provide a 50:50 solution, then Compound 1 was added and completely dissolved, followed by the addition of DCM to provide solutions in ratios of 87.5:12.5 and 86:14.

In Table 12, the DCM:DMSO (77:23) solvent system was prepared using the DCM:DMSO (65:35) HPMC E5 solution by adding additional DCM.

TABLE 11
HPMC E5 (mg/mL) Solubility

| Solvent System | HPMC E5 | Comments |
|---|---|---|
| THF 100% | 30 | Opaque |
| MeOH 100% | 30 | Opaque |
| EtOH-95 100% | 30 | Opaque |
| IPA 100% | 60 | Insoluble |
| DCM 100% | 60 | Opaque |
| Acetone | 30 | Opaque |
| Acetone:DCM (50:50) | 15 | Opaque |
| DCM:THF (33:67) | 20 | Opaque |
| DCM:MeOH (50:50) | 40 | Completely dissolved |
| DCM:MeOH (50:50) | 50 | Completely dissolved |
| DCM:MeOH (50:50) | 60 | Completely dissolved |
| DCM:MeOH (80:20) | 40 | Clear with some suspended |
| | | small/tiny particles |
| DCM:MeOH (87.5:12.5) | 15 | Dissolved very clear |
| DCM:MeOH (86:14) | 26 | Dissolved very clear |
| DCM:EtOH-95 (80:20) | 40 | Clear with some suspended |
| | | small/tiny particles |
| DCM:EtOH-95 (50:50) | 40 | Clear |
| DCM:IPA (50:50) | 30 | Clear with some suspended |
| | | small/tiny particles |
| DCM:EtOH-95 (50:50) | 30 | Dissolved but not as fast as in |
| | | DCM:MeOH (50:50) |
| DCM:EtOH-95 (86:14) | 20 | Clear with some suspended |
| | | small/tiny particles |
| DCM:DMSO (50:50) | 109 | Completely dissolved |
| DCM:DMSO (65:35) | 50 | Completely dissolved |
| DCM:DMSO (80:20) | 50 | Clear with some suspended |
| | | small/tiny particles |
| DCM:DMSO (95:5) | 50 | Opaque |

TABLE 12
SDI (Compound 1/HPMC E5/Poloxamer 407) Solubility

| | HPMC E5 | Pol 407 | | |
|---|---|---|---|---|
| Solvent System | (mg/mL) | (mg/mL) | (mg/mL) | Comments |
| DCM:MeOH (50:50) | 60 | 20 | 0 | Completely |
| | | | | dissolved |
| DCM:MeOH (50:50) | 60 | 20 | 120 | Insoluble |
| DCM:MeOH | 15 | 5 | 30 | Completely |
| (87.5:12.5) | | | | dissolved |
| DCM:MeOH (50:50) | 50 | 0 | 70 | Insoluble |
| DCM:MeOH (86:14) | 26 | 0 | 35 | Completely |
| | | | | dissolved |
| DCM:EtOH-95 | 50 | 0 | 63 | Insoluble |
| (50:50) | | | | |
| DCM:EtOH-95 | 20 | 0 | 29 | Clear with some |
| (86:14) | | | | suspended small/ |
| | | | | tiny particles |
| DCM:DMSO (65:35) | 50 | 0 | 115 | Completely |
| | | | | dissolved |
| DCM:DMSO (77:23) | 33 | 0 | 77 | Completely |
| | | | | dissolved |

### Solids Content in the Spray Drying Solution

The maximum amount of acceptable solids (i.e., the solubility of the combined starting materials) in the spray drying solution for large scale manufacture was evaluated in different solvent systems. The HPMC E5 polymer was added to the solvent system followed by the addition of Compound 1b with stirring at RT. The solubility was evaluated visually after 30 minutes and after leaving the sample at rest for periods of up to 72 hours.

As shown in Tables 13 to 16, the amount of acceptable solids in solution was dependent on time, the amount of each starting material and the solvent system.

As shown in Table 13, the SDI used in the solvent system was prepared in two steps using the procedure for SDI Sample 28, described in Example 6, below.

As shown in Table 14, the SDI used in the solvent system was prepared in two steps using the procedure for SDI Sample 28. In the DCM:MeOH system, a combination of Compound 1b (3 gms) and HPMC E5 (2 gms) was dissolved (5% w/v) in a 100 mL volume of the system. The term “NT” represents “Not Tested,” the term “VSS” represents “Very Slight Sedimentation” and the term “SS” represents “Slight Sedimentation.”

As shown in Table 16, a combination of Compound 1 b (6 gms) and HPMC E5 (4 gms) was dissolved (10% w/v) in DCM:DMSO (50:50) (100 mL). The term “VSS” represents “Very Slight Sedimentation” and the term “SS” represents “Slight Sedimentation.”

As shown in Table 15, a combination of Compound 1 b (4.5 gms) and HPMC E5 (3 gms) was dissolved (7.5% w/v) in DCM:DMSO (65:35) (100 mL). The term “SS” represents “Slight Sedimentation.”

TABLE 13
Solids Content (2.5% w/v)

| Time | DCM:EtOH-95 (80:20) | DCM:MeOH (87.5:12.5) |
|---|---|---|
| 0 min | Practically dissolved | Completely dissolved |

TABLE 14
Solids Content (5% w/v)

| | | DCM:EtOH-95 | DCM:MeOH | DCM:DMSO | DCM:DMSO |
|---|---|---|---|---|---|
| Time | | (87.5:12.5)* | (87.5:12.5)* | (50:50) | (65:35) |
| 0 | min | Partially dissolved | Practically | Completely | Completely |
| | | (cloudy solution) | dissolved | dissolved | dissolved |
| 15 | min | SS | VSS clear | NT | NT |
| | | | solution | | |
| 3 | h | SS | VSS clear | NT | NT |
| | | | solution | | |
| 24 | h | SS (cloudy | VSS clear | NT | NT |
| | | under stirring) | solution | | |

TABLE 15
Solids Content (7.5% w/v)

| Time | | DCM:DMSO (50:50) | DCM:DMSO (65:35) |
|---|---|---|---|
| 0 | min | Practically dissolved | Practically dissolved |
| 15 | min | VSS clear solution | SS clear solution |
| 3 | hrs | VSS clear solution | SS clear solution |
| 24 | hrs | Practically, Completely | VSS clear solution |
| | | dissolved | |
| 24-72 | hrs | Completely dissolved | Completely dissolved |

TABLE 16
Solids Content (10.0% w/v)

| Time | | DCM:DMSO (50:50) |
|---|---|---|
| 0 | min | Practically dissolved |
| 15 | min | SS clear solution |
| 3 | hrs | SS clear solution |
| 24 | hrs | Practically, |
| | | Completely dissolved |
| 24-72 | hrs | Completely dissolved |

### SDI-Surfactant Spray-Drying Process and Results

The Compound 1/polymer/surfactant co-precipitated SDI compositions were obtained by solid dispersion using the previously described spray-drying technique. The process parameters were set to conditions listed in Table 17. The composition of the SDI and yields are shown in Table 18.

In Table 17, the inlet temperature (In) and outlet temperature (Out) are shown in ° C., the atomization air pressure (Air) is shown in mm, the spray gas flow (Gas) is shown in L/h, the aspirator rate (Asp) and pump rate (Pmp) are shown in %, the volume flow (Vol) is shown in m³/h and the feed flow (Feed) is shown in mL/min. The spray gas flow was determined according to manufacturer's specifications and the actual feed flow was calculated by the volume of solution used according to pump run time.

TABLE 17
Spray-Drying Process Parameters

| Sample | | | | | | | | |
|---|---|---|---|---|---|---|---|---|
| No. | In | Out | Air | Gas | Asp | Vol | Pmp | Feed |
| 11 | 63-68 | 41-42 | 40 | 473 | 90 | 35 | 20-22 | 5-6 |
| 12 | 63-67 | 39-42 | 40 | 473 | 90 | 35 | 22 | 5-6 |
| 23 | 63-66 | 39-42 | 40 | 473 | 90 | 35 | 22 | 6-7 |
| 14 | 66-68 | 42-45 | 35 | 414 | 90 | 35 | 22 | 5-6 |
| 15 | 64-67 | 39-43 | 35 | 414 | 90 | 35 | 22 | 6-7 |
| 16 | 62-67 | 37-41 | 35 | 414 | 90 | 35 | 22 | 6-7 |
| 17 | 63-68 | 39-46 | 40 | 473 | 90 | 35 | 22 | 6-7 |
| 18 | 64-67 | 37-40 | 40 | 473 | 90 | 35 | 22 | 6-7 |
| 19 | 64-68 | 35-37 | 40 | 473 | 90 | 35 | 22 | 6-7 |
| 20 | 63-70 | 35-40 | 35 | 414 | 90 | 35 | 22 | 6-7 |

As shown in Table 1, the materials were dissolved into the solvent system, resulting in non-viscous completely transparent liquids. Samples 11, 12, 13, 14, 15, 16, 17 and 20 were prepared by dissolving the materials into the solvent mixture (200 mL).

For Samples 18 and129, the HPMC E5 was dissolved in 50:50 DCM:MeOH (25 mL:25 mL) mixture, then the surfactant was added, followed by the addition of neat DCM (150 mL) with gradual dispersion until the dissolution was complete.

SDI Samples from Samples 12 and 19 provided yields in a range of from about 78% to about 80%. SDI Samples from 14 and 16 with atomization air pressures of 35 mm (414 Normlitre/hour) provided yields in a range of from about 76% to about 77%.

The term “Yield_{ccv}” refers to the percentage (%) of the combined amounts recovered from the cyclone/collection vessel.

TABLE 18
SDI (Compound 1/Polymer/Surfactant) Composition

| Sample | | Cpd 1 | | Surfactant | | Yield_{ccv} |
|---|---|---|---|---|---|---|
| No. | Solvent System | (%) | Polymer (%) | (%) | | (%) |
| 11 | DCM:EtOH (80:20) | 60 | HPMC E5 (40) | None | | 74.2 |
| 12 | DCM:EtOH (80:20) | 60 | PVP K30 (30) | Pol 407 | (10) | 67.9 |
| 13 | DCM:EtOH (80:20) | 60 | PVP K30 (30) | Gel 50/13 | (10) | 67.6 |
| 14 | DCM:EtOH (80:20) | 60 | PVP K30 (38) | Pol 407 | (2) | 75.9 |
| 15 | DCM:EtOH (80:20) | 60 | PVP K30 (38) | Gel 50/13 | (2) | 76.9 |
| 16 | DCM:EtOH (80:20) | 60 | PVP K30 (38) | SLS | (2) | 70.5 |
| 17 | DCM:MeOH | 60 | HPMC E5 (30) | Pol 407 | (10) | 78.3 |
| | (87.5:12.5) | | | | | |
| 18 | DCM:MeOH | 60 | HPMC E5 (30) | Gel 50/13 | (10) | 80.1 |
| | (87.5:12.5) | | | | | |
| 19 | DCM:MeOH | 60 | HPMC E5 (38) | SLS | (2) | 78.3 |
| | (87.5:12.5) | | | | | |
| 20 | DCM:MeOH | 50 | PVP K30 (40) | Pol 407 | (10) | 72.2 |
| | (87.5:12.5) | | | | | |

### XRPD

The amorphous structure of the co-precipitated Surfactant-SDI from Samples 11, 12, 13, 14, 15, 16, 17, and 20 was evaluated by XRPD (results not shown). The XRPD pattern of each Sample was characteristic of an amorphous material.

### DSC

As shown in Table 19, DSC thermograms were obtained at a heating rate of 25° C./min. The endothermic inflection representing the glass transition temperature (GTT) was clearly observed only for the Samples 11, 14, 15, 16, and 19 between 85° C. and 105° C.

Exothermic transitions (Exo) were seen for Sample 11 and to a lesser degree for Samples 13, 14, 19, and 20. Endothermic transitions (Endo) were seen for Samples 12, 13, 15 and 17, with one melting peak observed for each Sample around 210° C., 207° C., 205° C., and 218° C. respectively.

The term “NO” represents “Not Observed” and the term “ND” represents “Not Determined.”

TABLE 19
DSC Results

| Sample | | | | | | |
|---|---|---|---|---|---|---|
| No. | GTT | HC | Exo | Ey | Endo | Ey |
| 11 | 100.6 | 0.605 | NO | ND | 199.3 | −0.72 |
| 12 | NO | ND | 136.3 | 8.12 | 207.9 | −22.08 |
| 13 | 84.8 | 0.188 | 115.7/ | 1.42/ | 205.7 | −23.32 |
| | | | 171.9 | 1.74 | | |
| 14 | 103.3 | 0.580 | 142.0 | 2.48 | 193.6/NO | −16.78/ND |
| 15 | 97.6 | 0.500 | NO | ND | 204.4 | −11.23 |
| 16 | 104.1 | 0.557 | NO | ND | NO | ND |
| 17 | NO | ND | NO | ND | 218.1 | −10.05 |
| 18 | NO | ND | NO | ND | 217.5 | −2.15 |
| 19 | 94.59 | 0.476 | NO | ND | 195.1 | −6.44 |
| 20 | NO | ND | 138.9 | 1.83 | NO/NO/200.9 | ND/ND/−25.84 |

Solubility of SDI-Surfactant Compositions in (5% w/w) Aqueous Solution

The solubility of the Compound 1/polymer/surfactant SDI compositions was determined in different aqueous media at 37° C. after 2 and 6 hours (see Tables 20 to 23). In each Table, T.C. represents the theoretical concentration of the solution based on sample weight assuming all materials are solubilized. The 72 hour results were visual observations only.

SDI compositions containing HPMC E5 showed higher and more stable solubility between 2 and 6 hours, both without surfactant and with Poloxamer 407 or Gelucire 50/13, providing concentrations between 387-436 μg/mL, representing 93.5-100% of the Theoretical Concentration (TC). The term “Theoretical Concentration” refers to the concentration of a solution in which all material is solubilized, as determined from the material weight.

For SDI compositions containing PVP K30 and Poloxamer 407 or Gelucire 50/13, the solubilized concentrations were between 225-446 μg/mL (representing 55.8-99.8% of the T.C.).

TABLE 20
Solubility (μg/mL) in
Water (400 μg/mL)

| Sample | | | | |
|---|---|---|---|---|
| No. | TC | 2 hrs | 6 hrs | 72 hrs |
| 11 | 412.8 | 0.0 | 0.0 | Cloudy |
| 12 | 415.2 | 0.0 | 0.0 | Cloudy |
| 13 | 420.0 | 0.0 | 3.1 | Cloudy |
| 14 | 412.8 | 0.0 | 0.0 | Cloudy |
| 15 | 410.4 | 0.0 | 0.0 | Cloudy |
| 16 | 432.0 | 0.0 | 0.0 | Cloudy |
| 17 | 417.6 | 0.0 | 0.0 | Cloudy |
| 18 | 415.2 | 0.0 | 0.2 | Cloudy |
| 19 | 405.6 | 0.0 | 0.0 | Cloudy |
| 20 | 421.4 | 0.8 | 1.0 | Cloudy |

TABLE 21
Solubility (μg/mL) in 1%
SLS in Water (400 μg/mL)

| Sample | | | | |
|---|---|---|---|---|
| No. | TC | 2 hrs | 6 hrs | 72 hrs |
| 11 | 408.0 | 106.7 | 41.0 | Cloudy |
| 12 | 410.2 | 5.7 | 5.4 | Cloudy |
| 13 | 420.0 | 6.2 | 5.6 | Cloudy |
| 14 | 403.2 | 6.0 | 5.1 | Cloudy |
| 15 | 405.6 | 7.4 | 6.2 | Cloudy |
| 16 | 400.8 | 7.4 | 6.4 | Cloudy |
| 17 | 417.6 | 12.1 | 7.5 | Cloudy |
| 18 | 422.4 | 18.6 | 8.9 | Cloudy |
| 19 | 427.2 | 37.7 | 12.2 | Cloudy |
| 20 | 458.0 | 7.5 | 6.3 | Cloudy |

TABLE 22
Solubility(μg/mL) in 5% Poloxamer
407 in Water (400 μg/mL)

| Sample | | | | |
|---|---|---|---|---|
| No. | TC | 2 hrs | 6 hrs | 72 hrs |
| 11 | 400.8 | 403.5 | 413.8 | Clear |
| 12 | 400.8 | 333.2 | 332.2 | Cloudy |
| 13 | 403.2 | 233.1 | 224.9 | Cloudy |
| 14 | 432.0 | 384.5 | 313.7 | Cloudy |
| 15 | 410.4 | 385.1 | 369.6 | Cloudy |
| 16 | 393.6 | 394.1 | 382.5 | Cloudy |
| 17 | 420.0 | 424.8 | 425.3 | Clear |
| 18 | 412.8 | 425.2 | 425.5 | Clear |
| 19 | 428.4 | 434.6 | 436.5 | Clear |
| 20 | 420.5 | 308.7 | 370.7 | Cloudy |

TABLE 23
Solubility (μg/mL) in 5% Gelucire
50/13 in Water (400 μg/mL)

| Sample | | | | |
|---|---|---|---|---|
| No. | TC | 2 hrs | 6 hrs | 72 hrs |
| 11 | 405.6 | 410.0 | 419.0 | Clear |
| 12 | 420.0 | 375.2 | 349.3 | Cloudy |
| 13 | 398.4 | 377.9 | 342.8 | Cloudy |
| 14 | 415.2 | 325.8 | 346.0 | Cloudy |
| 15 | 400.8 | 395.5 | 306.1 | Cloudy |
| 16 | 388.8 | 397.4 | 395.7 | Cloudy |
| 17 | 398.4 | 407.7 | 405.6 | Clear |
| 18 | 414.6 | 388.7 | 387.3 | Clear |
| 19 | 399.6 | 402.7 | 405.0 | Clear |
| 20 | 447.0 | 446.2 | 439.9 | Cloudy |

### Example 3

### Solubility of Selected Dry Blend Formulations

SDI Formulation Samples 4, 11, 12 and 17 were formulated with surfactants Microcrystalline Cellulose (MCC-102) or Poloxamer 407 (Pol 407) and a disintegrant Croscarmellose Sodium Type-A (CCS) (as shown in Table 24).

The term “internal phase” (IP) refers to excipient(s) incorporated into the SDI; e.g. the SDI is formed by spray drying Compound 1a or Compound 1b, a polymer and a surfactant in combination. The SDI containing the IP surfactant is subsequently mixed with the other ingredients shown in Table 24 to provide the dry blend formulation. The term “external phase” (EP) refers to a surfactant included in the dry blend formulation as with other optional excipients(s).

Each of the formulations were prepared by gentle dry blending using a mortar and pestle, then manually filling the powder into size 00 (0.91 mL) gelatin capsules for a total of 105 mg/cap.

The effect of the Poloxamer 407 surfactant in IP SDI Experiments 5 and e or in EP SDI Samples 1 and 3 used in formulations was compared with formulations where the surfactant was not present (Exp. 2 and 4).

TABLE 24
Dry Blend Formulations

| | | | | | Pol | | MCC- | |
|---|---|---|---|---|---|---|---|---|
| Exp. | SDI Sample | | Polymer | | 407 | | 102 | CCS |
| No. | No. (%) | | (%) | | (%) | | (%) | (%) |
| 1 | 4 | (52.2) | PVP K30 | (34.8) | EP | (10.0) | N/A | 3.0 |
| 2 | 4 | (52.2) | PVP K30 | (34.8) | N/A | | 10.0 | 3.0 |
| 3 | 11 | (52.2) | HPMC E5 | (34.8) | EP | (10.0) | N/A | 3.0 |
| 4 | 11 | (52.2) | HPMC E5 | (34.8) | N/A | | 10.0 | 3.0 |
| 5 | 12 | (52.2) | PVP K30 | (26.1) | IP | (8.7) | 10.0 | 3.0 |
| 6 | 17 | (52.2) | HPMC E5 | (26.1) | IP | (8.7) | 10.0 | 3.0 |

### Example 4

### Dissolution of Dry Blend Formulations

In vitro dissolution studies on dry blend formulations were carried out to determine whether a fed or fasted state had an effect on formulation solubility and dissolution rate.

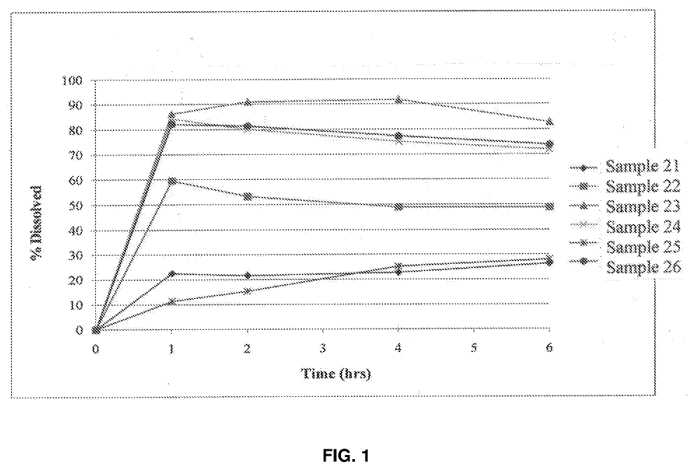

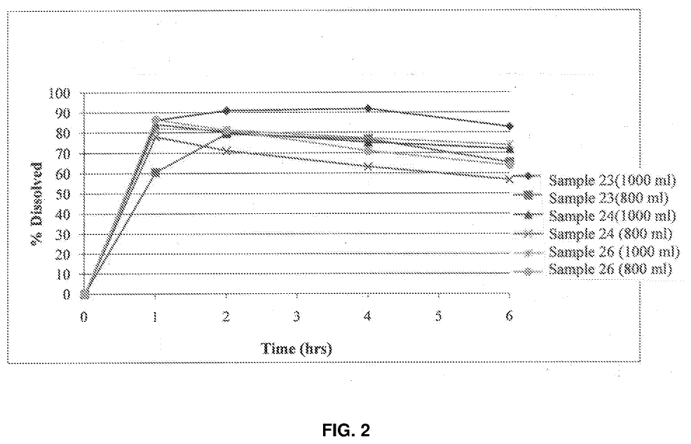

Capsules containing the formulations prepared according to the examples provided herein were dissolved in Fast State Simulated Gastric Fluid (FastSSGF) (FIGS. 1 and 2) using a USP Dissolution Apparatus II.

As shown in FIG. 1 capsules of dy blend formulation Samples 21, 22, 23, 24, 25 and 26 from Experiments 1-6, in Table 24, respectively, were dissolved in FastSSGF (1000 mL) at 0.1 N HCl in an aqueous solution of 1.5% SDS at a paddle speed of 100 revolutions per minute (RPM). By visual observation, the formulation Sample 21 was slightly turbid; Sample 22 was turbid; Sample 23 formed a slightly turbid suspension; Sample 24 had a very slightly turbid suspension; Sample 25 was turbid and Sample 26 formed a turbid suspension.

Formulation Samples 23, 24 and 26, demonstrated higher dissolution rates than Samples 21, 22, and 25. The highest dissolution rate, between 83 and 92% over one to six hours, was for Sample 23. The use of an IP surfactant showed no impact on Sample 26 dissolution but led to slower dissolution when is used in combination (IP or EP) in Samples 21, 22, and 25.

As shown in FIG. 2, Samples 23, 24, and 26 were dissolved in 800 mL FastSSGF at a paddle speed of 100 RPM and compared with the same Samples dissolved in 1000 mL FastSSGF.

Comparison of Samples 23, 24, and 26 in 800 and 1000 mL FastSSGF showed for all formulations that the dissolution rate decreased as the dissolution media volume decreased.

Overall, differences in release profiles were observed for certain formulations.

### Additional Results

After dissolution testing, precipitation of Samples 21, 22, and 25 was observed. Sample 22 and 25 precipitates were assessed by XRPD (data not shown). For Sample 25, the XRPD showed a partial recrystallization of Compound 1. For Sample 22, a formulation without Poloxamer surfactant, the XRPD showed that the amorphous form remained in the precipitate.

The XRPD patterns for each of the crystalline Compound 1a, crystalline Compound 1b and the surfactants and disintegrants used in the dry blend formulations were compared with the XRPD pattern of the partially recrystallized Sample 25. The comparison indicated that the crystalline peaks observed for Sample 25 did not correspond to the XRPD crystalline peaks for any of the materials used in the formulations. Without being bound by theory, the Sample 25 peaks may be due to interactions with the IP SDI surfactant or SLS from the in vitro dissolution media.

### Example 5

### SDI Formulation for Pharmacokinetic Studies

Formulations were prepared using SDI compositions with 52% loading in combination with MCC-102 or Pol 407 and CCS for rat PK studies. The SDI and excipients were sieved on a 30-mesh sieve prior to blending. Each of the formulations (1 gram) were prepared by gentle dry blending using a mortar and pestle. Formulation Samples 29 to 33 (see Table 25) and Samples 34 to 39 (see Table 26) were prepared for the PK studies.

TABLE 25
Formulation SDI Blends

| Sample | % | Polymer | | MCC-102^{c} | CCS^{c} | Pol 407 | |
|---|---|---|---|---|---|---|---|
| No. | w/w | (% w/w) | | (% w/w) | (% w/w) | (% w/w) | |
| 29 | 52.2 | PVP K30 | (34.8) | 10.0 | 3.0 | N/A | |
| 30 | 52.2 | HPMC E5 | (34.8) | 10.0 | 3.0 | N/A | |
| 31 | 52.2 | PVP K30 | (34.8) | N/A | 3.0 | EP | (10.0) |
| 32 | 52.2 | HPMC E5 | (34.8) | N/A | 3.0 | EP | (10.0) |
| 33 | 52.2 | HPMC E5 | (26.1) | 10.0 | 3.0 | IP | (8.7) |

TABLE 26
Formulation SDI Blends

| | SDI | | | MCC- | | |
|---|---|---|---|---|---|---|
| Sam- | Sample | | | 102 | | |
| ple | No. | Cpd 1 | Polymer | (% | CCS | Pol 407 |
| No. | (% w/w) | (% w/w) | (% w/w) | w/w) | (% w/w) | (% w/w) |
| 34 | 66.6 | 60% (40) | PVP K30 | 21.3 | 2.1 | 10.0 |
| | | | (27) | | | |
| 35 | 66.7 | 60% (40) | HPMC E5 | 30.3 | 3.0 | 0 |
| | | | (27) | | | |
| 36 | 33.3 | 60% (20) | PVP K30 | 54.2 | 2.5 | 10.0 |
| | | | (13) | | | |
| 37 | 33.3 | 60% (20) | HPMC E5 | 63.7 | 3.0 | 0 |
| | | | (13) | | | |
| 38 | 50 | 40% (20) | PVP K30 | 37.0 | 3.0 | 10.0 |
| | | | (30) | | | |
| 39 | 87.0 | 60% (52) | HPMC E5 | 10.0 | 3.0 | 0 |
| | | | (35) | | | |

In Table 27, the formulations at 52% loading were assayed by HPLC and found to have values between 95% and 100.6%. For Samples 34 and 36, due to agglomeration of the PVP K30, additional poloxamer and SDI was added to maintain Compound 1 loading for the 1 gram batch size. Sample 39 was more affected than 34. The same blending process as previously described was used.

TABLE 27
Assay Determination

| Sample | Avg | Individual |
|---|---|---|
| No. | (%) | (%) |
| 29 | 96.7 | 98.4, 95.0 |
| 30 | 99.3 | 99.3, 99.3 |
| 31 | 98.5 | 98.5, 98.6 |
| 32 | 100.3 | 100.6, 100.1 |
| 33 | 98.3 | 98.6, 98.0 |
| 34 | 97.5 | 97.8/97.1 |
| 35 | 99.4 | 98.9/100.0 |
| 36 | 87.1 | 87.3/86.9 |
| 37 | 95.6 | 95.8/95.3 |
| 38 | 94.7 | 94.6/95.9 |
| 39 | 99.0 | 99.0/99.0 |

### Example 6

### SDI Stability Studies

SDI Samples 27 and 28 were produced from crystalline Compound 1a using the same spray drying process steps and parameters that were used for Samples 11 to 20 for long term stability study (see Table 28).

Immediately after preparation (Time 0 hrs), SDI Samples 27 and 28 were assayed by XRPD and DSC. According to XRPD, both SDI samples had an amorphous nature. The DSC results shown in Table 29 were obtained at a heating rate of 10° C./min.

For the stability study, the bulk powder for each SDI Sample was packaged in double lined LDPE bags containing a desiccant (MiniPax MultiSorb™ desiccant packets 1 gram 50/50 AC/SG). The LDPE bags were then placed into closed HDPE bottles.

The samples were incubated under long term (25° C./60% RH) and accelerated (40° C./75% RH) stability conditions.

The term “NO” represents “Not Observed” and the term “ND” represents “Not Determined.”

TABLE 28
SDI Composition

| | | | Solvent |
|---|---|---|---|
| Sample | Cpd 1 | Polymer | System |
| No. | (% w/w) | (% w/w) | (% v/v) |
| 1 | 60 | PVP K30 | DCM:EtOH-100 |
| | | (40) | (80:20) |
| 27 | 60 | PVP K30 | DCM:EtOH-95 |
| | | (40) | (80:20) |
| 28 | 60 | HPMC E5 | DCM:MeOH |
| | | (40) | (87.5:12.5) |

TABLE 29
DSC Results

| Sam- | | | | | | |
|---|---|---|---|---|---|---|
| ple | | | | | | |
| No. | GTT | HC | Exo | Ey | Endo | Ey |
| 1 | 149 | 0.4 | NO | ND | — | — |
| 27 | 110 | 0.43 | NO | ND | 205 | −1.0 |
| 28 | 96/145 | 0.33/0.12 | NO | ND | 180/204/221 | −0.37/−0.35/ |
| | | | | | | −0.37 |

### Accelerated and Long Term Stability

### Non-Surfactant SDI XRPD

The non-surfactant SDI Samples 3 to 10 were exposed to 40° C./75% RH in open HPDE containers. At the one week and three week timepoint, XRPD showed that Samples 3 to 10 had no detectable signs of crystallization at an intensity scale of 100 counts at the 0 hours timepoint (data not shown). At the 6 week timepoint, SDI Samples 3 to 6 (containing PVP K30) showed signs of crystallization. The SDI Samples 7 to 10 (containing PVP K90 and HPMC E5) remained amorphous and had no detectable signs of crystallization at an XRPD intensity scale of both 1000 and 100 counts (data not shown). Similar results were obtained at the 12 week timepoint for SDI Samples 3 to 6 and 7 to 10 at 100 counts.

The non-surfactant SDI Samples 27 and 28 were exposed to 25° C./60% RH and 40° C./75% RH in closed bags/HPDE containers. At the four week and eight week timepoints under both conditions, no change in XRPD pattern was observed for SDI Samples 27 and 28 at an intensity scale of both 1000 and 100 counts (data not shown). At the 12 week timepoint for SDI Sample 28 at both 25° C./60% RH and 40° C./75% RH, no change in XRPD pattern was observed. At the 12 week timepoint for SDI Sample 27 at 25° C./60% RH, no change in the XRPD pattern was observed. At the 12 week timepoint for SDI Sample 27 at 40° C./75% RH, the XRPD pattern indicated partial recrystallization of the amorphous material (data not shown).

The SDI-Surfactant Composition Samples 12 to 20 were exposed to 40° C./75% RH in open HPDE containers.

At the 3 week 40° C./75% RH timepoint for Samples 12, 23, 14, 15, 18, and 20, the XRPD patterns indicated significant recrystallization of the amorphous material (data not shown). For Sample 17 minor crystallization had taken place. For Samples 16 and 19, the XRPD patterns showed that the Compositions remained amorphous.

At the 6 week 40° C./75% RH timepoint for Samples 16 and 19, the XRPD patterns showed that the Compositions remained amorphous at an intensity scale of both 1000 and 100 counts (data not shown). .

### Non-Surfactant SDI DSC

The DSC data shown in Tables 30 to 32 for samples of SDI Samples 3 to 10 placed in open HPDE containers and exposed to 40° C./75% RH was obtained at a heating rate of 10° C./min.

All SDI Samples showed several peaks over time, with new endothermic peaks for Samples using PVP K30 and HPMC E5 at 50% loading. Peak intensities (representing a general increase in enthalpy) increased depending on length of time on stability, packaging (open/closed containers) and stability conditions. The peak intensities were lower for HPMC E5 versus PVP K30, suggesting improved amorphous form stabilization.

The term “NO” represents “Not Observed,” the term “ND” represents “Not Determined,” the term “NS” represents “No Sample Available” and the term “D1” represents “Degradation above 170-180° C.”

TABLE 30
DSC Results 3 Week Open Cap

| Sam− | | | | | | |
|---|---|---|---|---|---|---|
| ple | | | | | | |
| No. | GTT | HC | Exo | Ey | Endo | Ey |
| 3 | NO | ND | NO | ND | 86/144/196 | −4.2/−77.2/−4.8 |
| 4 | 87 | 0.68 | NO | ND | 204/213 | −6.4/−15.5 |
| 5 | 90 | 0.64 | 161 | 4.46 | 213 | −33.1 |
| 6 | 130 | 0.35 | 167 | 58.8 | 95/220 | −4.3/−45.8 |
| 7 | 83 | 0.54 | NO | ND | 151 | −79.5 |
| 8 | 90 | 0.47 | NO | ND | 149/215 | −16.0/−23.0 |
| 9 | NO | ND | NO | ND | 87/127/219 | −3.2/−18.1/−1.3 |
| 10 | NO | ND | NO | ND | 90/106/222 | −4.2/−1.4/−1.0 |

TABLE 31
DSC Results 6 Week Open Cap

| Sam- | | | | | | |
|---|---|---|---|---|---|---|
| ple | | | | | | |
| No. | GTT | HC | Exo | Ey | Endo | Ey |
| 3 | 91 | 0.29 | NO | ND | 150 | −116.6 |
| 4 | NS | NS | NS | NS | NS | NS |
| 5 | NO | ND | NO | ND | 105/168/216 | −14.8/−10.5/−29.1 |
| 6 | NO | ND | 183 | 71.8 | 100/120/222 | −3.5/−2.8/−32.3 |
| 7 | 112 | 0.72 | NO | ND | 142 | −62.5 |
| 8 | 95 | 0.42 | NO | ND | 128/217 | −9.0/−25.7 |
| 9 | NO | ND | NO | ND | 93/136/157/219 | −2.7/−5.3/−1.5/−1.3 |
| 10 | 149 | 0.07 | NO | ND | 94/118/221 | −5.4/−1.8/−0.9 |

TABLE 32
DSC Results 12 Week Open Cap

| Sam- | | | | | | |
|---|---|---|---|---|---|---|
| ple | | | | | | |
| No. | GTT | HC | Exo | Ey | Endo | Ey |
| 3 | NO | ND | NO | ND | 87/101/128/ | −1.7/−2.3/−135.0/ |
| | | | | | 196/235 | −15.6/−1.0 |
| 5 | NO | ND | NO | ND | 95/138/215 | −3.6/−65.5/−31.5 |
| 9 | NO | ND | NO | ND | 87/157/218 | −7.5/−27.6/−2.5 |
| 10 | NO | ND | NO | ND | 91/144/170 | −6.8/−6.0/D1 |

TABLE 33
DSC Results 12 Week Closed Cap

| Sam- | | | | | | |
|---|---|---|---|---|---|---|
| ple | | | | | | |
| No. | GTT | HC | Exo | Ey | Endo | Ey |
| 3 | NO | ND | NO | ND | 85/96/120/198 | −2.5/−1.2/−118.7/−9.9 |
| 5 | NO | ND | NO | ND | 95/148/215 | −3.0/−37.6/−28.7 |
| 9 | NO | ND | NO | ND | 87/169/204/219/ | −6.0/−16.0/−0.3/−1.7/ |
| | | | | | 226 | −0.5 |
| 10 | NO | ND | NO | ND | 92/139/180 | −6.7/−11.7/D1 |

### Non-Surfactant SDI DSC

The stability results shown in Tables 34 to 36 were obtained under conditions at 40° C./75% RH in open containers. The inclusion of surfactants in the internal phase affected SDI stability as shown by the number and intensity of the DSC peaks. For SDI Sample 19, many thermal events above 200° C. indicated an increased degradation at the 3 week timepoint. For DSC thermograms obtained at either heating rates of 25° C./min (Table 34) or 10° C./min (Table 35 and 36), exothermic and endothermic enthalpies were significantly increased. In addition, SDI Samples 13 and 15 showed thermal events at temperatures below 40° C.

The term “NO” represents “Not Observed,” the term “ND” represents “Not Determined” and the term “D2” represents “Degradation above 190° C.”

TABLE 34
DSC Results 3 Week Open Cap

| Sam- | | | | | | |
|---|---|---|---|---|---|---|
| ple | | | | | | |
| No. | GTT | HC | Exo | Ey | Endo | Ey |
| 16 | NO | ND | 123 | 21.8 | 224 | −24.1 |
| 17 | 60 | 0.14 | NO | ND | 142/219 | −17.4/−44.6 |
| 81 | 110 | 0.24 | NO | ND | 92/142/D2 | −7.9/−5.9/D2 |

TABLE 35
DSC Results 3 Week Open Cap

| Sam- | | | | | | |
|---|---|---|---|---|---|---|
| ple | | | | | | |
| No. | GTT | HC | Exo | Ey | Endo | Ey |
| 11 | NS | NS | NS | NS | NS | NS |
| 12 | NO | ND | NO | ND | 48/124/208 | −10.9/−100.7/−26.4 |
| 13 | NO | ND | NO | ND | 38/60/133 | −3.6/−1.4/−125.2 |
| 14 | NO | ND | NO | ND | 43/149/205 | −1.6/−138.0/−13.9 |
| 15 | 36 | 0.14 | NO | ND | 155 | −148.0 |
| 16 | 130 | 0.14 | NO | ND | 86/160/200 | −2.9/−56.5/−16.9 |
| 17 | NO | ND | 108 | 12.9 | 123/219 | −17.3/−48.5 |
| 18 | NO | ND | NO | ND | 60/216 | −1.4/−46.9 |
| 19 | 120 | 0.4 | NO | ND | 86/185/D2 | −5.2/−12.1/D2 |
| 20 | NO | ND | NO | ND | 47/141 | −9.1/−110.1 |

TABLE 36
DSC Results 6 Week Open Cap

| Sam- | | | | | | |
|---|---|---|---|---|---|---|
| ple | | | | | | |
| No. | GTT | HC | Exo | Ey | Endo | Ey |
| 78 | NO | ND | 179 | 1.5 | 90/149/199 | −6.0/−46/−6.6 |
| 18 | NO | ND | NO | ND | 89/148/187 | −6.9/−15.0/−16.3 |

### SDI Composition DSC

SDI stability samples designated s and t, packaged in double lined LDPE bags containing a desiccant in closed HDPE bottles, were assayed at the 2, 4, 8 and 12 week timepoints after storage at 40° C./75% RH (see Tables 37 to 41) and at the 12 week timepoint after storage at 25° C./60% RH (see Table 42).

For SDI Sample 28, the DSC for the 2 week timepoint at 40° C./75% RH was comparable to the DSC for the 0 week timepoint. The DSC thermograms for the SDI Sample 28 samples stored at both 25° C./60% RH and 40° C./75% RH at the 12 week timepoint were also comparable to the DSC for the 0 week timepoint, but enthalpy values for those stored at 40° C./75% RH increased slightly on further storage.

For SDI samples, endothermic transitions increased with increased storage time, with a shift in the endothermic peaks to lower temperatures. The transitions were slightly higher in the samples stored at 40° C./75% RH.

SDI sample t was generally more stable than SDI Sample 28. The term “NO” represents “Not Observed”; the term “ND” represents “Not Determined.”

TABLE 37
DSC Results 0 Week

| Sam- | | | | | | |
|---|---|---|---|---|---|---|
| ple | | | | | | |
| No. | GTT | HC | Exo | Ey | Endo | Ey |
| 27 | 110 | 0.43 | NO | ND | 205 | −1.0 |
| 28 | 96/145 | 0.33/0.12 | NO | ND | 180/204/221 | −0.37/−0.35/−0.37 |

TABLE 38
DSC Results 2 Weeks

| Sam- | | | | | | |
|---|---|---|---|---|---|---|
| ple | | | | | | |
| No. | GTT | HC | Exo | Ey | Endo | Ey |
| 27 | 106 | 0.32 | NO | ND | 190/207 | −16.2/−4.8 |
| 28 | 87/185 | 0.4/0.16 | NO | ND | 219 | −0.41 |

TABLE 39
DSC Results 4 Weeks

| Sam- | | | | | | |
|---|---|---|---|---|---|---|
| ple | | | | | | |
| No. | GTT | HC | Exo | Ey | Endo | Ey |
| 27 | 106 | 0.23 | NO | ND | 165/175/206 | −8.7/−16.3/−6.4 |
| 281 | 156 | 0.14 | NO | ND | 92/180/220 | −2.4/−1.2/−1.1 |

TABLE 40
DSC Results 8 Weeks

| Sam- | | | | | | |
|---|---|---|---|---|---|---|
| ple | | | | | | |
| No. | GTT | HC | Exo | Ey | Endo | Ey |
| 27 | 85/99 | 0.38/0.16 | NO | ND | 122/207 | −31.4/−6.2 |
| 28 | 115 | 0.17 | NO | ND | 92/144/ | −5.2/−5.4/ |
| | | | | | 205/220 | −0.7/−2.3 |

TABLE 41
DSC Results 12 Weeks

| Sam- | | | | | | |
|---|---|---|---|---|---|---|
| ple | | | | | | |
| No. | GTT | HC | Exo | Ey | Endo | Ey |
| 27 | NO | ND | NO | ND | 82/102/138/ | −0.6/−3.1/−82.9/ |
| | | | | | 194/195 | −0.8/−4.4 |
| 28 | NO | ND | NO | ND | 93/151/220 | −4.6/−5.1/−2.4 |

TABLE 42
DSC Results 12 Weeks

| Sam- | | | | | | |
|---|---|---|---|---|---|---|
| ple | | | | | | |
| No. | GTT | HC | Exo | Ey | Endo | Ey |
| 27 | 87 | 0.37 | NO | ND | 141/206/231 | −75.2/−9.7/−0.5 |
| 281 | 81/115 | 0.35/0.22 | NO | ND | 146/220/235 | −6.5/−1.0/−0.5 |

### Appearance

The appearance of the SDI open cap samples stored at 40° C./75% RH was also examined at the 3 and 6 week timepoints (see Table 43). The color of the SDI was observed to change from a white powder to an off-white to yellow powder with attendant agglomeration for SDI-Surfactant Samples 13, 14, 12, 20 and 18. The degree of color change and agglomeration was in decreasing order from Samples 12, 15, 12, 20 and 21, with Sample 13 having the most and Sample 18 having the least. The Samples 13, 15 and 20 had exothermic peaks at the 0 week timepoint (see Table 19). In general, no visual color change has been observed in non-surfactant SDI Samples after 6 weeks at 40° C./75% RH.

TABLE 43
SDI Appearance

| Sample | | | 6 |
|---|---|---|---|
| No. | Excipients (%) | 3 Weeks | Weeks |
| 75 | PVP K30 (30)/Pol 407 (10) | Yellowish to off-white, | Same |
| | | agglomerates | |
| 76 | PVP K30 (30)/Gel 50/13 (10) | Yellowish, agglomerates | Same |
| 78 | PVP K30 (38)/Gel 50/13 (2) | Yellowish, agglomerates | Same |
| 81 | HPMC E5 (30)/Gel 50/13 (10) | Off-white, powder | Same |
| 83 | PVP K30 (40)/Pol 407 (10) | Off-white, powder | Same |

### Solubility and Water Content

At the 3 week 40° C./75% RH stability timepoint in open HPDE containers, the solubility of Samples 9 and 10 was evaluated (see Table 44). The evaluation in solution at 400 μg/mL and 37° C. was conducted at 2, 6 and 24 hours. Compared to solubility results at the 0 week stability timepoint (see Table 10), the solubility was not affected by the storage conditions.

At the 4 week 40° C./75% RH stability timepoint in open HPDE containers, the water content of the SDI Samples was determined by Karl Fischer titration (see Table 45). Samples 9 and 10 had a lower moisture content.

TABLE 44
SDI Solubility (μg/mL)

| Medium | | | |
|---|---|---|---|
| (5% in Water) | Time | 72 | 73 |
| SLS | TC | 402.2 | 415.2 |
| | 2 hrs | 320.7 | 328.0 |
| | 6 hrs | 297.6 | 302.9 |
| | 24 hrs | 235.1 | 224.1 |
| Pol 407 | TC | 398.6 | 399.0 |
| | 2 hrs | 362.3 | 321.5 |
| | 6 hrs | 387.6 | 386.1 |
| | 24 hrs | 398.3 | 394.9 |
| Gel 50/13 | TC | 423.4 | 399.6 |
| | 2 hrs | 406.4 | 374.1 |
| | 6 hrs | 412.6 | 396.8 |
| | 24 hrs | 412.6 | 397.6 |

TABLE 45
Water Content

| Sample | | | |
|---|---|---|---|
| No. | Cpd 1 (%) | Polymer | WC (%) |
| 3 | 50 | PVP K30 | 6.5 |
| 9 | 50 | HPMC E5 | 2.5 |
| 10 | 70 | HPMC E5 | 1.6 |

### Values for Assay, Water Content and Unknown Products

In general, the assay values (90-110%), the water content and the unknown product (i.e., degradation products or related substances) values (total unknown: ≤2.0%; single unknown: ≤0.2%) were each found to be within expected ranges. For SDI-Surfactant Samples, the amount of unknown products was generally found to be increased.

More specifically, the values under 40° C./75% RH stability conditions for SDI-Surfactant Samples 17, 18, and 19 in open containers at the 3 week timepoint (Table 46), the values for SDI-Surfactant Samples 16 and 19 in open containers at the 6 week timepoint (Table 46), the values for SDI Samples 27 and 28 in closed containers at the 8 week timepoint (Table 47) and the values for non-surfactant SDI Samples 4, 5, 9, and 10 in open and closed containers at the 12 week timepoint (Table 48) were each comparable to values at the 0 week timepoint and within expected ranges.

As previously indicated, the term “open containers” refers to placing the SDI in uncapped high density polyethylene (HDPE) bottles. Also, as previously indicated, the term “closed containers” refers to placing the SDI as a white powder in polyethylene (PE) bags with desiccant between the bags placed in capped HDPE bottles.

As shown in Table 47, no relative changes in assay or unknown product values under 40° C./75% RH stability conditions were observed for SDI Samples 27 and 28 in closed containers at stability timepoints from 0 to 12 weeks. The water content increased by about 2.0% for Sample 27 and by about 1.0% for Sample 28 from the 2 week to 12 week timeperiod.

As shown in Table 48, no changes in unknown product values under 40° C./75% RH stability conditions at the 12 month timepoint were observed for non-surfactant SDI Samples 3, 5, 9, and 10 in open and closed containers.

The relative assay and water content values for Samples 3 and 5 in open containers changed compared to the values in closed containers. These values for Samples 3 and 5 also changed compared to the values for Samples 9 and 10 under the same conditions at the 12 month timepoint.

The term “NT” represents “Not Tested”; the term “RRT” represents “Relative Retention Time.”

TABLE 46
Assay, Water Content and Unknown Values (%)

| Test | Week | 79 | 80 | 81 | 82 |
|---|---|---|---|---|---|
| Assay (%) | 0 | NT | NT | NT | NT |
| | 3 | NT | 99.1 | 99.6 | 99.9 |
| | 6 | 97.9 | NT | NT | 100.1 |
| Water (%) | 0 | NT | NT | NT | NT |
| | 3 | NT | 1.9 | 1.8 | 2.1 |
| | 6 | 1.9 | NT | NT | 1.9 |
| Total | 0 | NT | NT | NT | NT |
| Unknown | 3 | NT | 0.39 | 0.37 | 0.41 |
| (%) | 6 | 0.46 | NT | NT | 0.48 |
| Single | 0 | NT | NT | NT | NT |
| Unknown | 3 | NT | 0.14 | 0.13 | 0.14 |
| (%) | | | (RRT: 0.33) | (RRT: 0.33) | (RRT: 0.33) |
| | 6 | 0.19 | NT | NT | 0.20 |
| | | (RRT: 0.34) | | | (RRT: 0.34) |

TABLE 47
Assay, Water Content and Unknown Values (%)

| Test | Week | 90 | 91 |
|---|---|---|---|
| Assay (%) | 0 | 99.8 | 100.2 |
| | 2 @ 40/75 | 99.8 | 100.0 |
| | 4 @ 40/75 | 99.7 | 100.3 |
| | 8 @ 40/75 | 99.8 | 101.2 |
| | 12 @ 40/75 | 99.1 | 100.3 |
| | 12 @ 25/60 | 99.7 | 101.0 |
| Water (%) | 0 | 1.6 | 1.0 |
| | 2 @ 40/75 | 1.6 | 1.0 |
| | 4 @ 40/75 | 1.8 | 1.1 |
| | 8 @ 40/75 | 3.4 | 2.0 |
| | 12 @ 40/75 | 3.6 | 2.0 |
| | 12 @ 25/60 | 2.8 | 1.6 |
| Total Unknown (%) | 0 | 0.11 | 0.11 |
| | 2 @ 40/75 | 0.11 | 0.10 |
| | 4 @ 40/75 | 0.07 | 0.07 |
| | 8 @ 40/75 | 0.08 | 0.08 |
| | 12 @ 40/75 | 0.06 | 0.05 |
| | 12 @ 25/60 | 0.05 | 0.05 |
| Single Unknown (%) | 0 | 0.03 | 0.03 |
| | | (RRT: 0.86) | (RRT: 0.86) |
| | 2 @ 40/75 | 0.03 | 0.02 |
| | | (RRT: 0.86) | (RRT: 0.86) |
| | 4 @ 40/75 | 0.02 | 0.02 |
| | | (RRT: 1.16) | (RRT: 1.16) |
| | 8 @ 40/75 | 0.03 | 0.03 |
| | | (RRT: 0.86) | (RRT: 0.86) |
| | 12 @ 40/75 | 0.01 | 0.01 |
| | | (RRT: 1.16) | (RRT: 1.16) |
| | 12 @ 25/60 | 0.01 | 0.01 |
| | | (RRT: 1.16) | (RRT: 1.16) |

TABLE 48
Assay, Water Content and Unknown Values (%)

| Test | Week | 3 | 5 | 9 | 10 |
|---|---|---|---|---|---|
| Assay | 0 | NT | NT | NT | NT |
| (%) | 12 | 97.4 | 97.4 | 98.7 | 99.7 |
| | (open) | | | | |
| | 12 | 95.6 | 95.6 | 98.9 | 99.3 |
| | (closed) | | | | |
| Water | 0 | NT | NT | NT | NT |
| (%) | 12 | 4.3 | 4.3 | 2.8 | 1.9 |
| | (open) | | | | |
| | 12 | 5.7 | 5.7 | 2.8 | 1.8 |
| | (closed) | | | | |
| Total | 0 | NT | NT | NT | NT |
| Unknown | 12 | 0.11 | 0.11 | 0.11 | 0.11 |
| (%) | (open) | | | | |
| | 12 | 0.10 | 0.10 | 0.11 | 0.11 |
| | (closed) | | | | |
| Single | 0 | NT | NT | NT | NT |
| Unknown | 12 | 0.03 | 0.03 | 0.03 | 0.03 |
| (%) | (open) | (RRT: | (RRT: 0.86) | (RRT: 0.86) | (RRT: 0.86) |
| | | 0.86) | | | |
| | 12 | 0.03 | 0.03 | 0.03 | 0.03 |
| | (closed) | (RRT: | (RRT: 0.86) | (RRT: 0.86) | (RRT: 0.86) |
| | | 0.86) | | | |

### Results and Discussion

Spray drying is a convenient technique to prepare a coprecipitated SDI containing amorphous Compound 1 and PVP K30 or HPMC E5 polymers. The SDI containing HPMC E5 showed very good yields while those with PVP K30 and PVP K90 were lower due to agglomeration and fiber formation, respectively.

The XRPD patterns of all precipitated SDI samples were typical of amorphous material. No significant SDI weight loss was encountered at up to 125° C. in TGA.

DSC thermograms showed a glass transition peak between 85 and 105° C. and a melting peak between 158 and 224° C. depending on the amount and type of SDI formulation materials used.

For a PVP K30 SDI with Compound 1 loading from 70 to 80%, DSC exothermic transitions indicative of crystallization were seen. For a PVP K30 SDI with Compound 1 loading from 50% and 60%, no detectable exothermic transition was seen.

For a PVP K90 and HPMC E5 SDI, no detectable exothermic transitions were seen with the Compound 1 loading studied.

For a HPMC E5 SDI, DSC thermograms showed a glass transition peak between 85 and 105° C. and melting peaks between 194 and 218° C.

The use of either a PVP or HPMC polymer and optional surfactant in the SDI and the type of solvent system used were each shown to have an influence on solubility and Compound 1 loading.

In 5% aqueous solutions with a surfactant such as Gelucire 50/13, Poloxamer 407 (Lutrol F127) or SLS, the system maintained the SDI in solution for up to 2 hours. In particular, for a PVP K30 and PVP K90 SDI, the presence of a surfactant maintained SDI concentration at about 200 to 300 μg/mL in Gelucire 50/13 and Poloxamer 407 and to a certain extent in SDS. For the HPMC E5 SDI, the 5% aqueous surfactant solutions tested (except Poloxamer 188) were able to maintain SDI concentration at about 200 to 300 μg/mL.

The solvent systems that appeared to provide favorable solubility for the 10% w/v, 7.5% w/v and 5% w/v spray drying solutions include DCM:DMSO (50:50), DCM:DMSO (65:35) and DCM:MeOH (87.5/12.5) respectively. The DCM:MeOH solvent system appeared to provide a favorable dissolution profile for the HPMC E5 SDI with or without surfactant. Among the surfactants used with the HPMC E5 SDI, the Poloxamer 407 surfactant appeared to provide a favorable dissolution profile.

The SDI formed with polymers and optional surfactants using the described spray drying technique generally provided yields greater than 67%. The use of HPMC E5 (30-40% w/w) with or without surfactants provided yields from 78 to 80% and solubility concentrations in aqueous surfactants of between 387 to 436 μg/mL in a period of time between 2 and 6 hours. For a PVP K30 SDI with a surfactant such as Poloxamer 407 or Gelucire 50/13, the concentrations were between 225 to 446 pg/mL.

The in vitro dissolution in a fast state simulated gastric fluid medium for the HPMC E5 SDI with Compound 1 loading at 100 mg showed that between 80 to 90% of the SDI was dissolved in the first hour and remained stable for six hours.

For a non-surfactant SDI at the 3 week timepoint under 40° C./75% RH, XRPD showed no detectable Compound 1 crystallization peaks. For a surfactant SDI at the same timepoint, recrystallization peaks were seen in all the PVP K30/Gelucire and PVP K30/Poloxamer SDI Samples (12 to 15) and in the HPMC E5/Poloxamer SDI Sample 17. For the HPMC E5/Gelucire SDI Sample 18, minor crystallization peaks were observed. For the SLS SDI Samples 16 and 19, Compound 1 remained amorphous after the 3 and 6 week timepoints.

For PVP K30 and PVP K90 SDI Samples 4, 5, 6 and 8, with Compound 1 loading >60%, as a result of the DSC process, DSC thermograms showed heat-induced crystallization at the 0 week timepoint under 40° C./75% RH.

For the HPMC E5 SDI Sample 10 (70% loading) at the 3 week timepoint under 40° C./75% RH, DSC showed minor endothermic peaks corresponding to Compound 1 melting compared to the 0 week timepoint for the HPMC E5 SDI Sample 9 (50% loading).

For non-surfactant SDI Samples, DSC showed no significant changes after 6 weeks at 40° C./75% RH. For surfactant SDI Samples, an increased number and intensity of thermal events were observed. The endothermic peak enthalpies for surfactant SDI Samples containing either Gelucire and Poloxamer increased at the 3 week 40° C./75% RH stability timepoint. For SLS SDI Samples after 3 weeks at 40° C./75% RH, DSC showed thermal events above 200° C. at heating rates of both 25° C./min and 10° C./min, indicating degradation of the amorphous form, although equivalent peaks were not seen at the 6 week timepoint, most probably due to residual solvent evaporation. For Gelucire SDI Samples 13 and 15, DSC showed thermal events at stability temperatures below 40° C., along with observed SDI color changes.

The solubility of SDI Samples in various aqueous solutions did not appear to be affected by 40° C./75% RH stability conditions, remaining almost constant. At the 4 week timepoint at 40° C./75% RH, the PVP K30 SDI Sample 3 had become more hydroscopic compared to the HPMC E5 SDI Samples 9 and 10 (see Table 45).

For SDI Samples 17, 18, and 19 in open containers at the 0 week timepoint and at the 3 week timepoint at 40° C./75% RH, the assay values, water content and unknown product values were found to be within specification.

For SDI Samples 16 and 19 in open containers at the 0 week timepoint and at the 6 week timepoint at 40° C./75% RH, the assay values, water content and unknown product values were found to be within specification.

For non-surfactant SDI Samples 27 and 28 in closed containers at the 0 week timepoint and at the 2 and 4 week stability timepoints at 40° C./75% RH, the assay values, water content and unknown product values were found to be within specification with no signs of crystallization.

At the 6 week timepoint, DSC showed a general increase in exothermic and endothermic enthalpies. The PVP K30 SDI Sample 27 showed signs of crystallization in the XRPD patterns while the PVP K90 Sample 7 and HPMC E5 SDI Sample 28 remained amorphous. Similar results were obtained at the 12 week timepoint.

SDI Sample 27 (PVP K30) and Sample 28 (HPMC E5) at 60% Compound 1 loading remained physically and chemically stable at 40° C./75% RH (closed in PE bags with desiccant) for up 3 months at both 25° C./60% RH and 40° C./75% RH. After the 3 month timepoint at 40° C./75% RH, SDI Sample 27 had slight changes in the crystal structure as shown by XRPD with a total moisture increase of 2% compared to the HPMC E5 SDI Sample 28 moisture uptake of 1%.

### Example 7

### Lipid Capsule Preparation

The materials shown in Table 49 were used to prepare Formulation Sample 1 capsules containing 50 mg of Compound 1 (20.00% w/w dose loading). The term “TCQ” represents theoretical capsule quanitity (mg), the amount of each material per capsule. The term “TBQ” represents theoretical batch quanitity (kg), the amount of each material in the bulk product.

TABLE 49
Lipid Formulation

| Material | % w/w | TCQ | TBQ |
|---|---|---|---|
| Sieved Compound 1 (Passed through | 2.667 | 20.003 | 2.0003 |
| 10 mesh screen) | | | |
| Lauryl Macrogol-32 Glycerides | 49.869 | 374.018 | 37.4018 |
| (Gelucire 44/14), USP | | | |
| Macrogol 15 Hydrostearate, EP (Solutol | 47.458 | 355.935 | 35.5935 |
| HS-15) | | | |
| Butylated Hydroxytoluene NF, Tested | 0.006 | 0.045 | 0.0045 |
| NF/EP | | | |
| Total | 100.000 | 750.001 | 75.0001 |

To enable melting and elimination of all solid masses prior to dispensing, Gelucire 44/14 and Solutol HS-15, each in a closed container, were placed in a calibrated oven set at 70±5° C. for a minimum of 8 hours. An Olsa 150L Jacketed Mixing/Homogenizing Kettle was preheated to 70±5° C. for a minimum time period of about 15 minutes and the Gelucire 44/14 and Solutol HS-15 were each added to the Kettle via Vardex 1″ Tubing connected to a 1″ bottom powder inlet diaphragm valve under vacuum (between −0.10 and −0.51 bar).

The mixture was stirred for a time period of about 15 minutes using an Anchor Mixer (Operational range: 12-36 RPM) set at 24±12 RPM and Blade Mixer (Operational range: 22-69 RPM) set at 50±17 RPM to achieve a solution temperature of 70±5° C. The Kettle vacuum was released, the mixers were stopped and Butylated Hydroxytoluene was added to the solution. The mixers were started at the previous respective settings and the mixture was stirred at a temperature of 70±5° C. for a minimum time period of about 15 minutes or until the Butylated Hydroxytoluene was dissolved. A rinse volume (3 to 8 kg) of the solution was obtained. p Compound 1 was slowly added to the Kettle via the Vardex 1″ Tubing connected to a 1″ bottom powder inlet diaphragm valve under vacuum (between −0.10 and −0.51 bar). The rinse solution was used to flush the tubing and valve and the mixture was stirred using a Homogenizer Mixer (Operational range: 400-3000 RPM) set at 1700±1300 RPM and the Anchor Mixer set at 24±12 RPM. The vacuum was increased to between −0.51 and −1.02 bar and the mixture was stirred at the indicated respective settings for a minimum of 8 hours while maintaining a temperature of 70±5° C.

A recirculating pump and heat-traced transfer hoses were connected to a 3-way valve. The transfer hoses were heated and a temperature of 70±5° C. was maintained for a minimum time period of 15±0.5 minutes prior to recirculating the solution. The Kettle vacuum was released and, while maintaining a Kettle pressure of 0.45±0.25 bar and temperature of 70±5° C., the solution was recirculated and mixed using the Anchor Mixer set at 24±12 RPM and the Blade Mixer set at 50±17 RPM for a maximum time period of 60±0.5 minutes. Mixing and recirculation was stopped when a first solution sample analysis using an Olympus BX40 microscope configured for polarized light microscopy at magnification level 100× confirmed in at least 3 fields that Compound 1 was completely solubilized by the absence of crystals. In the event crystals are present in the first sampling, the solution is mixed and recirculated for a second maximum time period of 60±0.5 minutes. In the event crystals are present in a subsequent sampling, the solution is mixed and recirculated each time for a maximum time period of 60±0.5 minutes until at least 2 consecutive samplings confirm the absence of crystals. After sampling confirmed that Compound 1 was completely solubilized, while mixing and recirculating at the previous respective settings and pressure, the Kettle temperature was reduced to a temperature of 50±5° C. The Kettle temperature was maintained at a temperature of 50±5° C. and pressure of 0.45±0.25 bar while mixing and recirculating using the Anchor Mixer set at 20±5 RPM.

A capsule liquid filler (Shionogi brand) was prepared to fill size 00 gelatin capsules and was maintained at a temperature of 50±5° C. for a minimum time period of about 15 minutes prior to filling the hopper with the Kettle bulk solution. Capsules were filled until the bulk solution was exhausted, then cooled for a minimum time period of about 15 minutes and stored appropriately. The banding solution was prepared and placed in a sealed container in a calibrated oven set at a temperature of 55±5° C. for a minimum of 8 hours. The banding solution was placed in a capsule sealing machine (Shionogi brand) and maintained at a temperature of 45±10° C. The capsules were sealed at a seal roller speed of 125±75 RPM then stored appropriately.

### Example 8

### Preclinical In Vivo Oral Bioavailability Pharmacokinetic Rat Study

The exposure of encapsulated SDI Formulation Samples 28, 29, 30, 32 and encapsulated Lipid Formulation Sample 1 after oral administration to rats was evaluated. As shown in the preceding examples, the SDI Formulations and Lipid Formulation, based on the materials used in each formulation, is expected to differ in their dissolution properties. The oral bioavailability of the SDI Samples relative to each other and relative to the Lipid Formulation Sample were determined in this study.

As shown in Example 5, the SDI Sample Formulation capsules contained 52.2% w/w Compound 1. As shown in Example 7, the Lipid Formulation capsules contained 20% w/w Compound 1. The two SDI Samples 29 and 31 each contained PVP-K30 and two SDI Samples 30 and 32 each contained HPMC E5, as shown in Example 5, Table 25. Each SDI Sample was blended with either the surfactant Pol 407 (SDI Samples 31 and 32) or with MCC-102 (SDI Samples 29 and 30). CCS was added as a disintegrant in all formulations.

The capsules for each formulation were administered to 5 groups of male Sprague Dawley Crl:SD rats (Charles River, Kingston, N.Y.) (n=4 per group). The animals were provided normal chow and water ad libitum.

The doses administered correlated to about 350 mg in a 70 kg human, a clinically relevant dose. The dose amount (mg) administered was constant, but since the animals varied in weight, the dosage (mg/kg) varied across groups.

For example, the SDI Formulation capsules were dosed at 1.5 mg/animal (calculated at about 5 mg/kg for an average 300 g animal). The individual doses were then averaged across the group. The dose of the Lipid Formulation capsules was dosed based upon the weight of the individual animal to provide a delivered dose of 5 mg/kg.

At specified time points (0.5 hour, 1 hour, 3 hours, 6 hours, 9 hours, and 24 hours post-dose) plasma samples were obtained. Plasma concentration at specified times and the calculated pharmacokinetic parameters were compared among groups by analysis of variance (ANOVA) using SigmaStat 3.0. Noncompartmental pharmacokinetic parameters were determined using WinNonLin 5.2 (Pharsight Corporation, Carey N.C.) for each individual rat and then averaged across each dosing group.

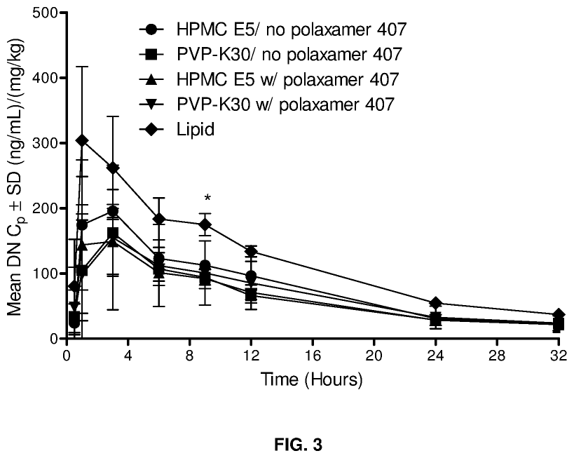

FIG. 3 shows the dose normalized plasma concentration for each formulation tested (final Group 1, 3, and 4 n=4 and final Group 2 and 5 n=3) as a function of time. The ratio of the plasma concentration relative to the dose at each time point was calculated for each animal and then averaged across the group.

The * indicates a p-value <0.05 (ANOVA, multiple comparisons vs. lipid-vehicle control) for the 9 hour sample results. The term “Cₚ” represents “Plasma Concentration,” the term “DN” represents “Dose-Normalized” and the term “SD” represents “Standard Deviation.”

### Results and Discussion

The time at which the maximum concentration was reached (Tₘₐₓ) was about 3 hours after dosing with the SDI Formulation Samples and about 1 hour after dosing with the Lipid Formulation. The Cₘₐₓ was highest in rats dosed with the Lipid Formulation.

Bioavailability tended to be higher in the HPMC E5 SDI Formulations, compared to the PVP K30 SDI Formulations even though a statistically significant p-value was not obtained. The groups dosed with the HPMC SDI (Groups 2 and 4), compared to those dosed with the PVP K30 SDI (Groups 1 and 3), showed a slightly higher Cₘₐₓ and area under the curve (AUC). The addition of a surfactant (Pol 407) in the external phase did not appear to improve SDI Formulation exposure.

Although the exposure of Compound 1 was higher when administered in the Lipid Formulation Sample 1 (for example a Lipid Formulation comprising 50% w/v Gelucire® 44/14, 50% w/v Solutol® HS and 2.668% w/v Compound 1) than when administered in the SDI Formulations, the bioequivalence of any of the SDI Formulations at 52.2% dose loading, compared to the Lipid Formulation at 20% dose loading, demonstrate that the SDI formulations significantly improve solubility and consequent bioavailability of a higher dose loaded formulation compared to the variable solubility and precipitation when the SDI is solubilized in Lipid-based Formulation Samples 2 (100% load; amorphous form, no polymer), 3 (50% load; PVP K30 SDI), 4 (60% load; PVP K30 SDI), 5 (70% load; PVP K30 SDI), 6 (80% load; PVP K30 SDI), 7 (50% load; PVP K90 SDI), 8 (70% load; PVP K90 SDI), 9 (50% load; HPMC E5 SDI) and 10 (70% load; HPMC E5 SDI), at high dose loads (see Tables 7, 8 and 10 and associated discussion).

### Example 9

### Preclinical In Vivo Oral Bioavailability Pharmacokinetic Rat Study

The exposure of encapsulated SDI Formulation Samples 34, 35, 36, 37 and 39 and encapsulated Lipid Formulation Sample 38 after oral administration to rats was evaluated.

As shown in Table 50, the Compound 1a crystalline form was used to prepare the amorphous form with the polymer and materials listed. The SDI Formulation Samples were dry blended according to procedures described in the Examples herein. For example, POL 407 was blended with the SDI in the external phase to the granules. The purity of Compound 1a was 86.1%, resulting in a 1.3 mg dose administered to each animal compared to a target dose per animal of 1.5 mg. The term “N/A” represents “Not Applicable.”

TABLE 50
Study Formulations

| | Dose | | | | | |
|---|---|---|---|---|---|---|
| | Load | | | Pol 407 | MCC-102 | CCS |
| Group | (% w/w) | Polymer | % w/w | (% w/w) | (% w/w) | (% w/w) |
| 1 | 40 | PVP-K30 | 27 | 10 | 21.3 | 2.1 |
| 2 | 40 | HPMC E5 | 27 | N/A | 30.3 | 3.0 |
| 3 | 20 | PVP-K30 | 13 | 10 | 54.2 | 2.5 |
| 4 | 20 | HPMC E5 | 13 | N/A | 63.7 | 3.0 |
| 6 | 52.2 | HPMC E5 | 35 | N/A | 10.0 | 3.0 |

As shown in Table 51, the Lipid Formulation was prepared using the amorphous SDI at a dose load of 60% in a mixture with the materials listed. The term “N/A” represents “Not Applicable.”

TABLE 51
Group 5 Lipid Formulation Sample 1

| | Dose | |
|---|---|---|
| | Load | |
| Materials | (% w/w) | % w/w |
| Amorphous SDI | 2.67 | 2.86 |
| Gelucire ® 44/14 (Lauroyl Macrogol-32 Glycerides USP) | N/A | 49.87 |
| Solutol ® HS 15 (Macrogol-15-Hydroxystearate EP) | N/A | 47.46 |
| Total fill weight (rounded to whole mg) | | 100 |

The capsules for each formulation were administered to 6 groups of male Sprague Dawley Crl:SD rats (Charles River, Portage, Mich.) (n=4 per group). The animals were provided normal chow and water ad libitum.

For example, the SDI Formulation capsules were dosed at 1.5 mg/animal (calculated at about 6 mg/kg for an average 250 g animal). The individual doses were then averaged across the group. The dose of the Lipid Formulation capsules was dosed based upon the weight of the individual animal to provide a delivered dose of 5 mg/kg.

At specified time points (0.5 hour, 1 hour, 3 hours, 6 hours, 9 hours, 24 hours, 32 hours, 48 hours post-dose) plasma samples were obtained. Plasma concentration at specified times and the calculated pharmacokinetic parameters were compared among groups by analysis of variance (ANOVA) using SigmaStat 3.0. Noncompartmental pharmacokinetic parameters were determined using WinNonLin 5.2 (Pharsight Corporation, Carey N.C.) for each individual rat and then averaged across each dosing group.

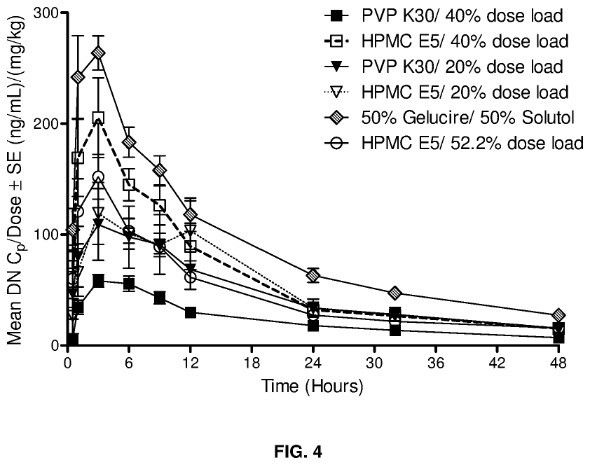

FIG. 4 shows the dose normalized plasma concentration for each formulation tested as a function of time. The ratio of the plasma concentration relative to the dose at each time point was calculated for each animal and then averaged across the group.

The term “Cₚ” represents “Plasma Concentration,” the term “DN” represents “Dose-Normalized” and the term “SD” represents “Standard Deviation.”

### Results and Discussion

The Tₘₐₓ tended to be earliest after dosing for the Lipid Formulation, followed by the HPMC E5 SDI Formulations at 40% and 52% dose loading. However, when comparing the Tₘₐₓ for all six groups by ANOVA, differences among the groups did not reach significance.

When comparing the dose-normalized maximal plasma concentration (Cₘₐₓ) among the six groups, Group 1 (PVP K30 SDI at 40% dose load) was significantly reduced when compared to that of Group 5 (Lipid Formulation); the Cₘₐₓ in the other groups were not significantly less that that measured in animals dosed with the Lipid Formulation.

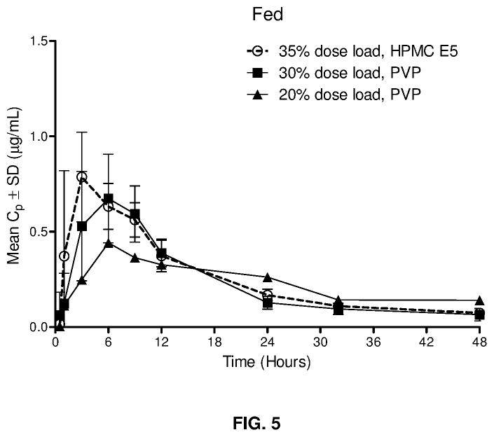

While the dose normalized AUC of all SDI Formulation groups was significantly less than that of the Lipid Formulation Group 5, as shown in FIG. 5, the AUC plasma concentration of Compound 1 was generally higher when administered in the HPMC SDI Formulations (20%, 40%, or 52.2% dose load) than in the PVP K30 SDI Formulations and statistically significant (ANOVA; All Pairwise Multiple Comparison Procedure; Holm-Sidak method).

For example, Group 2 (HPMC E5 SDI at 40% dose load) was significantly higher than of Group 1 (PVP K30 SDI at 40% dose load); Group 4 (HPMC E5 SDI at 20% dose load) was significantly higher than that of Group 1 (PVP K30 SDI at 40% dose load); Group 6 (HPMC E5 SDI at 52% dose load) was significantly higher than that of Group 1 (PVP K30 SDI at 40% dose load); and Group 3 (PVP K30 SDI at 20% dose load) was more than that of Group 1 (PVP K30 SDI at 40% dose load).

In another example, Group 1 and Group 2 (PVP K30 SDI at 40% dose load and HPMC E5 SDI at 40% dose load, respectively), when compared by a Student's t-test, the absolute and dose-normalized Cₘₐₓ values were higher for the HPMC E5 SDI Formulations. In general, the HPMC E5 SDI Formulation exposure was higher than the PVP K30 SDI Formulation exposure, with the HPMC E5 SDI Formulation at 40% dose loading having the highest exposure.

The half-life and mean residence time among the six groups showed no significant differences. Other comparisons did not reach significance.

### Example 10

### Preclinical In Vivo Oral Bioavailability Pharmacokinetic Food Effect Study

The food effect of encapsulated granule SDI Formulation Samples 40, 41, and 42 after oral administration to rats fasted from normal chow or rats fed high fat chow was evaluated.

The Compound 1a crystalline form was used to prepare the amorphous form with the polymer. The SDI Formulation Samples 40 and 41 were dry blended (both without surfactant) and granulated according to procedures described in the Examples herein and encapsulated. In particular, the SDI Formulation Blend Sample 42 granules (with surfactant) were prepared according to the procedure of Example 12.

As shown in Table 52, the capsules for each formulation were administered to 6 groups of male Sprague Dawley Crl:SD rats (Charles River, Portage, Mich.) (n=4 per group). The animals in respective groups were provided high fat chow (34.9% fat), normal chow (4.3% fat). Water was provided ad libitum.

The percent fat in the high fat diet is similar to those typically utilized for human clinical high fat diets (50-60% of calories from fat; see the FDA Guidance for Industry: Food-Effect Bioavailability and Fed Bioequivalence Studies, Food and Drug Administration, Rockville, Md.).

Of the six groups, Groups 1, 3 and 5 were fed normal chow for two days, fasted overnight prior to administration, then allowed to eat four hours after dosing. Groups 2, 4 and 6 were fed high fat chow for two days and allowed to eat ad libitum prior to administration.

TABLE 52
Study Formulations

| | Dose Load | | |
|---|---|---|---|
| Group | (% w/w) | Polymer | % w/w |
| 1 | 35 | HPMC E5 | 23 |
| 2 | 35 | HPMC E5 | 23 |
| 3 | 30 | PVP-K30 | 20 |
| 4 | 30 | PVP-K30 | 20 |
| 5 | 20 | PVP-K30 | 60 |
| 6 | 20 | PVP-K30 | 60 |

The capsules were dosed at 1.5 mg/animal (calculated at about 5 mg/kg for an average 300 g animal). The individual doses were then averaged across the group. The dose of the Lipid Formulation capsules was dosed based upon the weight of the individual animal to provide a delivered dose of 5 mg/kg.

At specified time points (0.5 hour, 1 hour, 3 hours, 6 hours, 9 hours, 24 hours, 32 hours, 48 hours post-dose) plasma samples were obtained. The significance of plasma concentration differences and the calculated pharmacokinetic parameters were compared among groups by analysis of variance (ANOVA) using SigmaStat 3.0. Pharmacokinetic parameters were determined using WinNonLin 5.2 (Pharsight Corporation, Carey N.C.) for each individual rat and then averaged across each dosing group. Plasma concentration at specified times and the calculated pharmacokinetic parameters were compared between the fed and fasted status by a Student's t-test using Excel.

FIG. 5 shows the dose normalized plasma concentration for each formulation tested as a function of time in fed animals. The ratio of the plasma concentration relative to the dose at each time point was calculated for each animal and then averaged across the group.

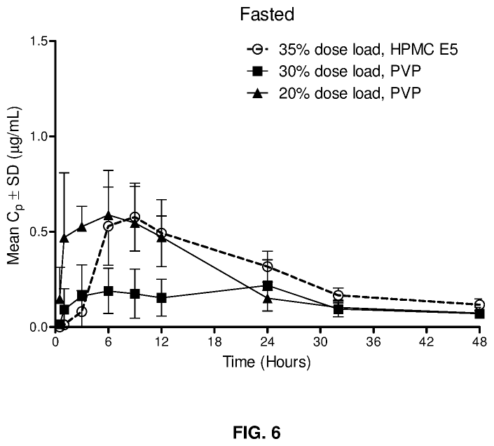

FIG. 6 shows the dose normalized plasma concentration for each formulation tested as a function of time in fasted animals. The ratio of the plasma concentration relative to the dose at each time point was calculated for each animal and then averaged across the group.

The term “Cₚ” represents “Plasma Concentration,” and the term “SD” represents “Standard Deviation.”

### Results and Discussion

The pharmacokinetic parameters for the SDI Formulations used in this study were not significantly different as defined by the AUC for each animal. Additionally, there were no significant differences in exposure between the fed groups.

For the encapsulated HPMC E5 SDI Formulation, there were no significant differences in exposure and food effect between the fed and fasted groups.

Comparing the exposure in fasted animals, the exposure of the encapsulated HPMC E5 SDI Formulation was higher than that of the PVP K30 SDI Formulation at 30% dose loading.

For the encapsulated PVP K30 SDI Formulation at both 20% and 30% dose loading, the exposure in fed animals was significantly higher than the exposure in fasted animals as measured by the dose normalized Cₘₐₓ and AUC.

Comparing the exposure of the PVP K30 SDI Formulation at 20% and 30% dose loading, the exposure of the 30% dose load Formulation was significantly lower than the exposure of the 20% dose load Formulation. However, the PVP K30 SDI Formulation at both dose loads showed a significant food effect as defined by Cₘₐₓ.

### Example 11

### Preclinical In Vivo Oral Bioavailability Pharmacokinetic Food Effect Study

As prepared in Table 53, the food effect of encapsulated SDI Formulations Sample A, Sample B, Sample C and Sample D (200 mg) was compared to encapsulated Lipid Formulation Sample E (60 mg) after oral administration to pentagastrin treated dogs. Samples A, B, C, D, and E correspond to Groups 1-5, respectively, in Table 53.

As shown in Table 53, the dose load represents 60% w/w SDI. The dose administered to each dog was based on an average animal weight of 12 kg (range 8 to 16 kg). The actual dose administered was calculated using the body weight of each individual animal.

TABLE 53
Study Formulations

| | | Dose | Dose | Dosage | |
|---|---|---|---|---|---|
| Group | Diet | (mg) | Load (%) | (mg/kg) | Polymer (%w/w) |
| 1 | Fed | 200 | 35%^{d} | 16.6 | HPMC E5 (23.3) |
| 2 | Fasted | 200 | 35%^{d} | 16.6 | HPMC E5 (23.3) |
| 3 | Fed | 200 | 30% | 16.6 | PVP K30 (20) |
| 4 | Fasted | 200 | 30% | 16.6 | PVP K30 (20) |
| 5 | Fasted | 60 | 2.86% | 5 | 50% Gelucire ® 44/14: 50% |
| | | | | | Solutol ® HS 15 |

As shown in Table 54, each material in each Lipid Formulation capsule is shown as Amount per capsule (Amt) (mg) and % w/w.

TABLE 54
Lipid Formulation

| Materials | Amt (mg) | % w/w |
|---|---|---|
| Amorphous SDI | 60 | 2.67 |
| Gelucire ® 44/14 (Lauroyl Macrogol-32 | 1122 | 49.87 |
| Glycerides USP) | | |
| Solutol ® HS 15 | 1068 | 47.46 |
| (Macrogol-15-Hydroxystearate EP) | | |
| Total fill weight (rounded to whole mg) | 2250 | 100 |

The capsules for each formulation were administered to 5 groups of non-naïve male Beagle Dogs (Stillmeadow Inc., Sugar Land, Tex.) (n=4 per group). Both fed and fasted (overnight) dogs were pretreated 40 minutes prior to administration with 6 mg/kg pentagastrin (i.m.). The fed dogs received high-fat canned feed 30 minutes prior to capsule administration. The fasted dogs were fed 4 hours after administration. All dogs were acclimated to the high fat food for at least 6 days prior to the study.

Because the pH of the stomach of fasted dogs is variable among individual dogs and can reach as high as pH 8.0 (Kararli, T T. Comparison of the gastrointestinal anatomy, physiology, and biochemistry of humans and commonly used laboratory animals. Biopharm. Drug. Disps. 1995, 16: 351-380; Akimoto M, Nagahata N, Furuya A, Fukushima K, Higuchi S, Suwa T. Gastric pH profiles of beagle dogs and their use as an alternative to human testing. Eur J Pharm Biopharm. 2000, 49:99-102), the study was done with pentagastrin-pretreated dogs. The pH in fasted human stomachs is approximately 2 (Kararli, 1995). Pentagastrin is an analog of the hormone gastrin and stimulates gastric acid secretion so that the gastric pH is more representative of a fasted human subject (Kararli, 1995; Akimoto et al., 2000). However, the use of pentagastrin standardizes the model, reduces any variability due to differences in the gastric pH of individual dogs, and makes the gastric pH more similar to that of humans. The dose of pentagastrin (6 μg/kg intramuscularly 40 minutes prior to oral dosing) was used based upon published studies (Kararli, 1995; Akimoto et al., 2000).

Previous published work has shown that using a high-fat diet in fed vs. fasted dog studies may be predictive of a human food effect (Lentz K A, Quitko M, Morgan D G, Grace J E Jr, Gleason C, Marathe P H. Development and validation of a preclinical food effect model. J Pharm Sci. 2007, 96:459-472; Homer L M, Clarke C R, Weingarten A J. Effect of dietary fat on oral bioavailability of tepoxalin in dogs. J Vet Pharmacol Ther. 2005, 28:287-291).

For example, the SDI Formulation capsules were dosed at 200 mg (calculated at about 6.7 to about 10 mg/kg for an average 10 to 15 kg animal).

At specified time points, (30 minutes, 1 hour, 2 hours, 4 hours, 6 hours, 8 hours, 12 hours, and 24 hours post-dose) plasma samples were obtained. Plasma concentration at specified times and the calculated pharmacokinetic parameters were compared among groups by analysis of variance (ANOVA) using SigmaStat 3.0. Noncompartmental pharmacokinetic parameters were determined using WinNonLin 5.2 (Pharsight Corporation, Carey N.C.) for each individual animal and then averaged across each dosing group.

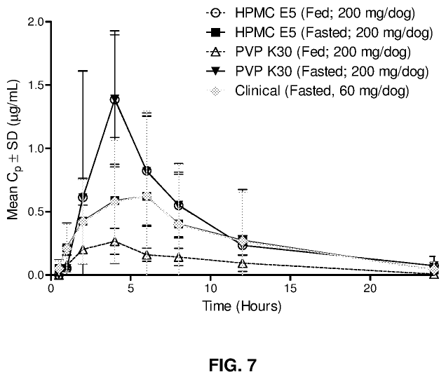

FIG. 7 shows the average plasma concentration for each formulation tested as a function of time. The ratio of the plasma concentration relative to the dose at each time point was calculated for each animal and then averaged across the group.

The term “Cₚ” represents “Plasma Concentration” and the term “SD” represents “Standard Deviation.”

### Results and Discussion

The exposure of Compound 1 was higher in the fasted dogs when administered any of the SDI Formulations using PVP K30 or HPMC E5 and the Lipid Formulation. The fed dogs using the PVP K30 SDI showed a markedly reduced exposure, thus demonstrating that a food effect exists when the PVP K30 SDI is used. In contrast, exposure of Compound 1 using the HPMC E5 SDI in either the fasted or fed state was surprisingly similar, thus demonstrating that the food effect is avoided when the HPMC E5 SDI is used.

### Example 12

### Clinical In Vivo Lipid Capsule Oral Bioavailability Food Effect Study

Compound 1 has been evaluated in a Phase 1, randomized, placebo-controlled, escalating, single-dose, safety, tolerability, PK, and food effect study in healthy adult volunteers. Compound 1 was provided in lipid-filled gelatin capsules. The primary objective of the study was to determine a dose range for Compound 1 that safely achieved pharmacologically active target plasma concentrations (as determined from xenograft studies) and that was appropriate for use in a subsequent multiple-dose study.

Subjects in the study were enrolled in 2 stages. In Stage 1, 40 subjects were accrued in 5 cohorts of 8 subjects with each cohort receiving a sequentially higher single dose of Compound 1 at dose levels of 0.03, 0.1, 0.3, 1, and 3 mg/kg. Within a cohort, 6 subjects (3 males and 3 females) received Compound 1 and 2 subjects (1 male and 1 female) received placebo. An additional 12 subjects (6 males and 6 females) were enrolled in Stage 2 to evaluate the effect of food on the PK of Compound 1.

During both Stages 1 and 2 of the study, data regarding adverse events, vital signs, blood counts, coagulation assessments, blood chemistry determinations, urinalyses, and ECGs were collected at baseline and repeatedly over 72 hours after administration of the study medication, and again at a follow-up visit 7 days after the last study treatment. In both Stages 1 and 2, blood samples for assessment of plasma Compound 1 concentrations were collected at multiple timepoints. Compound 1 concentrations were analyzed using LC-MS/MS, validated for human plasma. Blood for measurement of plasma DHODH levels are collected at multiple time points. Plasma DHODH concentrations are analyzed using a clinically validated ELISA (R&D Systems). Similarly, blood for measurement of plasma VEGF-A levels was collected at multiple time points. Plasma VEGF-A concentrations were analyzed using a clinically validated ELISA (R&D Systems).

As planned, 40 subjects (20 males and 20 females) completed their participation in Stage 1 of the study, and 12 subjects (6 males and 6 females) completed their participation in Stage 2 of the study. Subject ages ranged from 20 to 55 years (Stage 1) and 18 to 52 years (Stage 2). Their body weights ranged from 51 to 98 kg (Stage 1) and 59 to 85 kg (Stage 2).

Compound 1 was generally well tolerated and there were no serious drug-related adverse events. Among the 40 subjects dosed in Stage 1, the most frequent treatment-emergent adverse events were headache (9 episodes in 8 subjects, all receiving Compound 1) and nausea (5 episodes in 5 subjects, 4 receiving Compound 1 and 1 receiving placebo). Other types of adverse events occurred in fewer than 5 subjects (10%). During Stage 2, the most frequent adverse events were headaches (3 episodes in 3 subjects) and back pain (2 episodes in 2 subjects); other adverse events were noted only in single subjects. All adverse events were Grade 1 in severity, except 1 case of Grade 2 diarrhea in a subject receiving 1 mg/kg of Compound 1 in the fasted state in Stage 2. The incidence, relationship to study drug, and severity of adverse events were not clearly dose dependent, although the number of headaches may have increased slightly with dose. No deaths or serious adverse events occurred during the study. No subject prematurely terminated the study for safety reasons.

In both stages, there were no safety concerns based on subjects' physical examinations, vital sign measurements, or ECGs. No clinically significant changes in hematology, coagulation, or chemistry parameters were observed. Similarly, no clinically meaningful urinalysis abnormalities were seen.

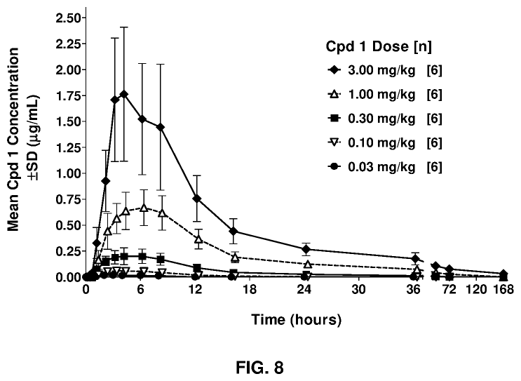

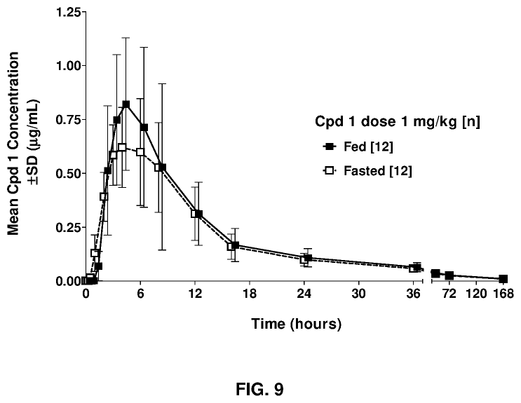

Mean plasma concentration-time profiles for Compound 1 during Stage 1 are shown in FIG. 8. Mean plasma concentration-time profiles for Compound 1 according to fed or fasted status of subjects are shown in FIG. 9. Compound 1 appeared in plasma after a lag time of about 30 minutes. At doses 0.30 mg/kg, Compound 1 concentrations persisted in plasma through 72 hours and, at the 3.0-mg/kg dose, low concentrations of Compound 1 were still evident at 168 hours after drug administration. The mean Cₘₐₓ was increased in subjects when they received the drug after a high-fat, high-calorie meal. With or without food, target plasma concentrations established in animal tumor models were safely achievable.

PK parameters for Compound 1 in plasma indicate a mean Tₘₐₓ in the range of 3 to 6 hours. During Stage 1, mean values for Cₘₐₓ and AUC rose steadily with dose. Increases in mean Cₘₐₓ values were generally dose proportional. Increases in mean AUC₀₋₂₄ values were somewhat greater than dose proportional through the 1.00-mg/kg dose level and then less than dose proportional in the transition from the 1.00-mg/kg to the 3.00-mg/kg dose levels. The terminal half-life (t_{1/2β}) was in the range of 28 to 56 hours.

The ingestion of a high-fat, high-calorie meal just prior to administration of 1 mg/kg of Compound 1 in Stage 2 increased the mean Cₘₐₓ by about 40% but did not materially change other PK parameters.

During Stage 1, Cₘₐₓ values were marginally higher (p=0.043, ANOVA) for females relative to males, but AUC₀₋₂₄ values were not significantly different. During Stage 2, Cₘₐₓ and AUC₀₋₂₄ values were higher for females than for males (p<0.01 for both comparisons, ANOVA). The relevance of these differences in this study is not clear given that similar sex-related differences were not observed in a subsequent Phase 1 multiple-dose study (data not shown).

DHODH is evaluated in the plasma samples from subjects enrolled in the 3-mg/kg group (in Stage 1). The mean changes from baseline in the Compound 1 group are similar to those in the placebo group over the course of the sampling period.

### Example 13

### PVP Tablet Preparation

The materials shown in Table 55 were used to prepare Blend Formulation Sample 42 for use in Formulation Sample 42a tablets containing 25 mg of Compound 1 (20% w/w dose loading), Formulation Sample 42b tablets containing 100mg of Compound 1 (20% w/w dose loading) and Formulation Sample 42c tablets containing 200 mg of Compound 1 (20% w/w dose loading).

TABLE 55
PVP Formulation Blend Sample 42

| Item | Material | w/w % |
|---|---|---|
| A* | Compound 1 SDI (Cpd 1:PVP 40:60) | 50.0 |
| B* | Microcrystalline Cellulose type 102 NF | 17.0 |
| C | Lactose Monohydrate 80 NF | 11.5 |
| D | Sodium starch glycolate NF | 2.5 |
| E | Magnesium Stearate NF | 0.5 |
| F** | Microcrystalline Cellulose type 102 NF | 7.0 |
| G** | Lactose Monohydrate 80 NF | 7.0 |
| H** | Sodium starch glycolate NF | 2.5 |
| I** | Poloxamer 188 Prilled NF | 1.0 |
| J** | Colloidal Silicon Dioxide NF | 0.5 |
| K** | Magnesium Stearate NF | 0.5 |
| Total | | 100 |

* At weighing step, adjust quantity as needed to maintain w/w %
**Adjust weighing quantity according to granule yield to maintain w/w %

Materials A-K were weighed and sieved in the following order: A, C, D, E and B, using a FitzMill equipped with a 30 mesh screen. The sieved materials were loaded into a PK 1ft³ V-blender and mixed for a time period of about 5 minutes at 25 RPM. The resulting dry blend was granulated using a roller-compactor TFC-Labo at a compaction pressure of 500±100 psi, a target roll speed of about 1.75 RPM (in a range of from about 1.25 RPM to about 2.00 RPM), a target screw speed of about 21 RPM (in a range of from about 16 RPM to about 25 RPM) and a target gap thickness of about 0.055 inches (in a range of from about 0.05 inches to about 0.07 inches). Uncompacted powder was collected then recirculated back into the roller compactor hopper. The internal-phase ribbons were collected then reduced to granules using a FitzMill equipped with a 30 mesh screen. The weight of materials F, G, H, I, J and K were adjusted according to the granule yield to maintain w/w % then sieved in the following order: G, H, I, J, K and F, using a FitzMill equipped with a 30 mesh screen. The sieved materials were loaded into a PK 1 ft³ V-blender and mixed for a time period of about 5 minutes at 25 RPM. The bulk granulation batch was added to the PK 1ft³ V-blender and mixed with the sieved materials for a time period of about 10 minutes at 25 RPM.

For Formulation Sample 42a 25 mg tablets, a Mini-Press II Tablet Press was prepared with a 6 mm round standard concave B-Tooling punch size. Tablets were compressed to obtain an average target weight for 10 tablets of 1250 mg (in a range of from about 1188 mg to about 1313 mg), a target individual tablet weight of 125 mg (in a range of from about 112.5 mg to about 137.5 mg), a target individual thickness of 4.5 mm (in a range of from about 3.5 mm to about 5.5 mm) and a target individual hardness of 5 kp (in a range of from about 3 kp to about 8 kp).

For Formulation Sample 42b 100 mg tablets, a Mini-Press II Tablet Press was prepared with a 11 mm round standard concave B-Tooling punch size. Tablets were compressed to obtain an average target weight for 10 tablets of 5000 mg (in a range of from about 4750 mg to about 5250 mg), a target individual tablet weight of 500 mg (in a range of from about 450 mg to about 550 mg), a target individual thickness of 5.7 mm (in a range of from about 4.7 mm to about 6.7 mm) and a target individual hardness of 7 kp (in a range of from about 5 kp to about 9 kp).

For Formulation Sample 42c 200 mg tablets, a Mini-Press II Tablet Press was prepared with a 18.97×9.91 mm oblong standard concave B-Tooling punch size. Tablets were compressed to obtain an average target weight for 10 tablets of 10000 mg (in a range of from about 9500 mg to about 10500 mg), a target individual tablet weight of 1000 mg (in a range of from about 900 mg to about 1100 mg), a target individual thickness of 7.6 mm (in a range of from about 6.6 mm to about 8.6 mm) and a target individual hardness of 13.5 kp (in a range of from about 11 kp to about 16 kp).

### Example 14

### Clinical In Vivo PVP Tablet Oral Bioavailability Food Effect Study

In various clinical trials, the Compound 1 safety profile using a Lipid Formulation has shown that the 1.5 mg/kg dose tested in various dose amounts (25 mg, 50 mg, 100 mg, 125 mg and 200 mg), and prepared as described above, has been acceptable at doses up to and including 1000 mg (the highest single dose tested). However, the Compound 1 dose loading that has been achievable in the Lipid Formulation capsule has limited the dose strength that can be chronically delivered at acceptable dosage form amounts, where each dose of the Lipid Formulation capsule requires the administration of at least six capsules per dose.

The PVP SDI Formulation tablets prepared in Example 13 were compared to the Lipid Formulation capsules prepared in Example 7 in a clinical BA/BE (bioequivalence/bioavailability) study that evaluated the effect of food on the bioavailability of the PVP SDI Formulation tablets.

Compound 1 was administered as a single-dose in Lipid Formulation capsules or as PVP SDI Formulation tablets. The primary objective of the study was to determine the comparative single-dose PK and safety profiles of Compound 1 administered in the 2 formulations. The study was also aimed to study of the food effect on the PK and safety profiles for the PVP SDI Formulation tablet.

Subjects in the study were enrolled in 3 stages. In Stage 1, 24 subjects were randomly accrued into 3 cohorts of 8 subjects receiving Compound 1 in both the Lipid Formulation capsule and the PVP SDI Formulation tablet at dose levels of 0.5 mg/kg, 1 mg/kg, and 2 mg/kg. In Stage 2, 24 subjects were accrued in 3 cohorts of 8 subjects with each cohort receiving a sequentially higher doses of Compound 1 in the PVP SDI Formulation tablet at dose levels of 400, 800, and 1600 mg. An additional 12 subjects (6 males and 6 females) were enrolled in Stage 3 to evaluate the effect of food on the PK of Compound 1 when administered at 400 mg and 1000 mg in the PVP SDI Formulation tablet.

During the study, data regarding adverse events, vital signs, blood counts, coagulation assessments, blood chemistry determinations, urinalyses, and ECGs were collected at baseline and repeatedly over 72 hours after administration of the study medication, and again at a follow-up visit 7 days after the last study treatment. Blood samples for assessment of plasma Compound 1 concentrations were collected at multiple timepoints. Compound 1 concentrations were analyzed using LC-MS/MS, validated for human plasma.

As planned, 24 subjects (12 males and 12 females) completed their participation in Stage 1 of the study, 24 subjects (12 males and 12 females) completed their participation in Stage 2 of the study, and 12 subjects (6 males and 6 females) completed their participation in Stage 3 of the study. Subject ages ranged from 22 to 54 years (Stage 1), 19 to 47 years (Stage 2), and 20 to 50 years (Stage 3).

Compound 1 was generally well tolerated. The overall safety profile is consistent with the observations in the previous Compound 1 clinical studies. In Stage 1, sporadic episodes of dry mouth, abdominal discomfort, headache, and diarrhea were observed; in Stage 2, sporadic episodes of nausea, anorexia, and abdominal distention were observed; in Stage 3, sporadic episodes of ocular discomfort, nasal congestion, and cough were observed. Events were mostly mild. No deaths or serious adverse events occurred during the study.

In all 3 stages, there were no safety concerns based on subjects' physical examinations, vital sign measurements, or ECGs. In general, no clinically significant changes in hematology, coagulation, chemistry, or urinalysis parameters were observed. In Stage 1, 1 male subject who received 2 mg/kg of Compound 1 in the Lipid Capsule Formulation in Week 1 was incidentally found to have a Grade 3 elevation of serum creatine kinase at the check-in for the Week 2 study period. The abnormal value was considered unlikely to be drug related in view of a history of strenuous activity that likely resulted in release of creatine kinase from muscle. However, as a precautionary measure, the subject was excluded from further participation in the study.

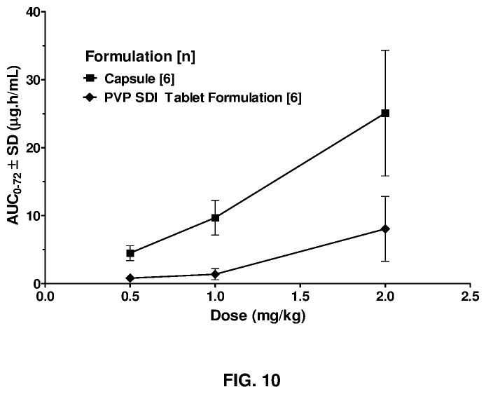

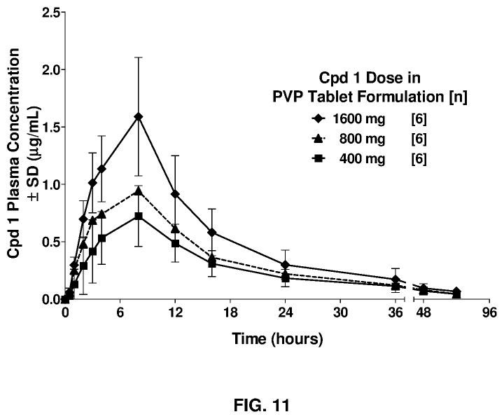

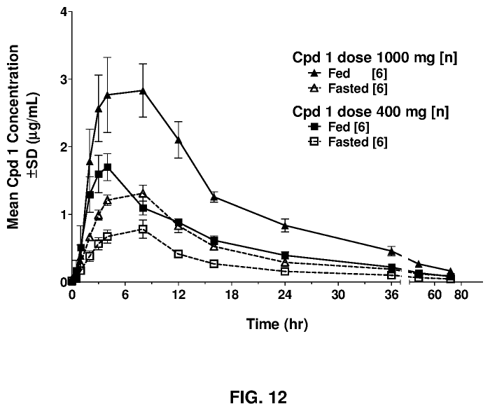

Mean plasma concentration-time profiles for Compound 1 during Stage 1 with the Lipid Capsule Formulation were similar to plasma-concentration profiles observed in previous Phase 1a studies. As shown in FIG. 10, across the 3 administered dose levels, the PVP SDI Tablet Formulation showed average Cₘₐₓ and AUC₀₋₂₄ values that were 19% and 18%, respectively, of those obtained with the Lipid Capsule Formulation. Mean plasma concentration-time profiles for Compound 1 at higher doses in the solid formulation are shown in FIG. 11. Compound 1 appeared in plasma after a lag time of about 30 minutes. Compound 1 concentrations persisted in plasma through 72 hours and were still evident at 168 hours after drug administration. The mean Cₘₐₓ was increased in subjects when they received the drug after a high-fat, high-calorie meal as shown in FIG. 12.

PK parameters for Compound 1 in plasma demonstrated a mean Tₘₐₓ in the range of 3 to 7 hours. During Stage 1, the relative bioavailability of Compound 1 in the PVP SDI Formulation tablet ranged between 14 to 28%, indicating a significant difference in the bioequivalence of Compound 1 between the PVP SDI Formulation tablet and Lipid Formulation capsules. During Stage 2, when only the PVP SDI Formulation tablet was administered, mean values for Cₘₐₓ and AUC rose with dose. However, the increases in mean Cₘₐₓ values were not dose proportional (p<0.05 for both comparisons, ANOVA). The half-life was in the range of 38 to 65 hours. As was also demonstrated, ingestion of a high-fat, high-calorie meal just prior to administration of 400 mg or 1000 mg of Compound 1 in Stage 3 increased the mean Cₘₐₓ and AUC by about 100%.

### Example 15

### HPMC 50mg Tablet Preparation

The materials shown in Table 56 were used to prepare Formulation Sample 43 tablets containing 50 mg of Compound 1 (33.33% w/w dose loading).

TABLE 56
50 mg Tablet Formulation Sample 43

| Item | Material | w/w % |
|---|---|---|
| A* | Compound 1 SDI (Cpd 1:HPMC E5 60:40) | 55.55 |
| B | Microcrystalline Cellulose, NF | 28.00 |
| | (Avicel PH-102) | |
| C* | Lactose Monohydrate, NF (FlowLac 100) | 10.95 |
| D | Croscarmellose Sodium,NF | 4.00 |
| E | Colloidal Silicon Dioxide, NF | 1.00 |
| F | Magnesium Stearate, NF | 0.40 |
| G** | Magnesium Stearate, NF | 0.10 |
| Total | | 100 |

* At weighing step, adjust quantity as needed to maintain w/w %)
**Adjust weighing quantity according to granule yield to maintain w/w %)

Materials A-G were weighed and sieved in the following order: A, B, C, D and E, using a FitzMill equipped with a 20 mesh screen at a speed of 70%. The sieved materials were loaded into a PK 1 ft³ V-blender and mixed for a time period of about 5 to about 10 minutes at 25 RPM. Material F was manually sieved through a 30 mesh screen then added to the PK 1ft³ V-blender and mixed for a time period of about 2 minutes at 25 RPM. The resulting dry blend was compacted to form ribbons using a roller-compactor TFC-Labo at a compaction pressure of 1000±100 psi, a target roll speed of 2.5 RPM (in a range of from 2.00 RPM to 3.00 RPM), a target screw speed of 37.5 RPM (in a range of from 30.0 RPM to 45.0 RPM) and a target ribbon thickness of 1.0 mm (in a range of from 0.8 mm to 1.3 mm). Uncompacted powder was collected then manually sieved through a 30 mesh screen and recirculated back into the roller compactor hopper. The ribbons were collected then reduced to granules using a FitzMill equipped with a 20 mesh screen at a speed of 70%. Material G was manually sieved through a 30 mesh screen and loaded into the PK 1ft³ V-blender with the bulk granulation batch. The materials were mixed for a time period of about 2 minutes at 25 RPM.

A Mini-Press II Tablet Press was prepared with a 9/32 inch round standard concave B-Tooling punch size. Tablets were compressed to obtain an average target weight for 10 tablets of 1500 mg (in a range of from about 1425 mg to about 1575 mg, or from about 1425 mg to about 1575 mg), a target individual tablet weight of 150 mg (in a range of from about 135 mg to about 165 mg, or from about 135 mg to about 165 mg), a target individual thickness of 3.8 mm (in a range of from about 3.4 mm to about 4.2 mm, or from about 3.4 mm to about 4.2 mm) and a target individual hardness of 8 kp (in a range of from about 4 kp to about 12 kp, or from about 4 kp to about 12 kp).

### Example 16

### HPMC 200 mg Tablet Preparation

The materials shown in Table 57 were used to prepare Formulation Sample 44 tablets containing 50 mg of Compound 1 (33.33% w/w dose loading).

TABLE 57
200 mg Tablet Formulation Sample 44

| Code | Material | w/w % |
|---|---|---|
| A* | Compound 1 SDI (Cpd 1:HPMC E5 60:40) | 55.55 |
| B | Microcrystalline Cellulose, NF (Avicel PH- | 28.00 |
| | 102) | |
| C* | Lactose Monohydrate, NF (FlowLac 100) | 10.95 |
| D | Croscarmellose Sodium,NF | 4.00 |
| E | Colloidal Silicon Dioxide, NF | 1.00 |
| F | Magnesium Stearate, NF | 0.40 |
| G** | Magnesium Stearate, NF | 0.10 |
| Total | | 100 |

*At weighing step, adjust quantity as needed to maintain w/w %)
**Adjust weighing quantity according to granule yield to maintain w/w %)

Materials A-G were weighed and sieved in the following order: B, C, D, E and F, using a FitzMill equipped with a 20 mesh screen at a speed of 70%. The sieved materials were loaded into a PK 1 ft³ V-blender and mixed for a time period of about 5 to about 10 minutes at 25 RPM. Material F was manually sieved through a 30 mesh screen, added to the PK 1ft³ V-blender and mixed for a time period of about 2 minutes at 25 RPM. The resulting dry blend was compacted to form ribbons using a roller-compactor TFC-Labo at a compaction pressure of 1000±100 psi, a target roll speed of 2.5 RPM (in a range of from 2.00 RPM to 3.00 RPM), a target screw speed of 37.5 RPM (in a range of from 30.0 RPM to 45.0 RPM) and a target ribbon thickness of 1.0 mm (in a range of from 0.8 mm to 1.3 mm). Uncompacted powder was collected then manually sieved through a 30 mesh screen and recirculated back into the roller compactor hopper. The ribbons were collected then reduced to granules using a FitzMill equipped with a 20 mesh screen at a speed of 70%. Material G was manually sieved through a 30 mesh screen and loaded into the PK 1ft³ V-blender with the bulk granulation batch. The materials were mixed for a time period of about 2 minutes at 25 RPM.

A Mini-Press II Tablet Press was prepared with a 15/32 inch round standard concave B-Tooling punch size. Tablets were compressed to obtain an average target weight for 10 tablets of 6000 mg (in a range of from about 5700 mg to about 6300 mg, or from about 5820 mg to about 6180 mg), a target individual tablet weight of 600 mg (in a range of from about 540 mg to about 660 mg, or from about 564 mg to about 636 mg), a target individual thickness of 5.8 mm (in a range of from about 5.4 mm to about 6.2 mm, or from about 5.5 mm to about 6.1 mm) and a target individual hardness of 14 kp (in a range of from about 9 kp to about 19 kp, or from about 10 kp to about 18 kp).

### Example 17

10 mg (Sample 45), 25 mg (Sample 46), and additional 50 mg tablets (Sample 47) were produced from 50 mg, 125 mg, or 250 mg per tablet, respectively, of a PVP blend formulation of the same composition described in Table 55 of Example 13, above, using a similar roller compaction procedure as described for the 50 and 200 mg tablets in Examples 15 and 16, above.

5 mg tablets (Sample 48) were produced by roller compaction of the 12.5% Compound 1 (40%) SDI formulation shown in Table 58, below, using a similar procedure to that described in Example 15 for the 50 mg tablets. 100 mg of the PVP Blend shown in Table 58 was used for each tablet.

TABLE 58
PVP Blend for 5 mg Roller Compacted Tablets

| Item | Material | w/w % |
|---|---|---|
| A | Compound 1 SDI (Cpd 1:PVP 40:60) | 12.5 |
| B | Microcrystalline Cellulose type 102 NF | 35.0 |
| C | Lactose Monohydrate 80 NF | 31.0 |
| D | Sodium starch glycolate NF | 2.5 |
| E | Magnesium Stearate NF | 0.5 |
| F | Microcrystalline Cellulose type 102 NF | 7.0 |
| G | Lactose Monohydrate 80 NF | 7.0 |
| H | Sodium starch glycolate NF | 2.5 |
| I | Poloxamer 188 Prilled NF | 1.0 |
| J | Colloidal Silicon Dioxide NF | 0.5 |
| K | Magnesium Stearate NF | 0.5 |
| Total | | 100 |

An additional batch of 5 mg tablets (Sample 49) was produced by direct compation of a PVP blend of the composition shown in Table 59, below. Items A-F of that formulation were sieved together using a 30-mesh sieve and mixed for 5 minutes using a V-blender. The final ingredient, magnesium stearate was sieved, added to the mix and blended for another 2 minutes. 100 mg of the PVP blend shown in Table 59 was used for each tablet.

TABLE 59
PVP Blend for 5 mg Direct Compacted Tablets

| Item | Material | w/w % |
|---|---|---|
| A | Compound 1 SDI (Cpd 1:PVP 40:60) | 12.5 |
| B | Microcrystalline Cellulose type 102 NF | 42.0 |
| C | Lactose Monohydrate 80 NF | 38.0 |
| D | Sodium starch glycolate NF | 5.0 |
| E | Poloxamer 188 Prilled NF | 1.0 |
| F | Colloidal Silicon Dioxide NF | 0.5 |
| G | Magnesium Stearate NF | 1.0 |
| Total | | 100 |

All of the tablets were compressed. The 5 and 25 mg tablets were compressed with 6 mm diameter round concave tooling, while the 10 and 50 mg tablets were compressed with 5 and 8 mm diameter round concave tooling, respectively.

More than 300 tablets were produced of each of Samples 45 to 49, above. Ten tablet were randomly chosen and evaluated for appearance, weight, thickness, hardness, friability, and time of disintegration. The tablets were found to be uniform in weight, thickness, and hardness, with no physical defects observed. The tablets containing 50% SDI (Samples 45-46) disintegrated between 9 to 18 minutes while those containing 12.5% SDI disintegrated within one minute.

Stablility studies were conducted on 10 mg and 50 mg tablets (Samples 45 and 47) above by storing the samples in bottles at either 15° C. at 60% relative humidity or at 40° C. at 75% relative humidity for 18 months. The samples were tested at 1, 3, 6, 9, 12, and 18 months. No increase in degradation products or other signs of deterioration were observed in any of the samples. The water content in all of the samples remained below 4% throughout the study. Dissolution profiles were also essentially unchanged.

The invention is not to be limited in scope by the specific aspects described herein. Indeed, various modifications of the invention in addition to those described will become apparent to those skilled in the art from the foregoing description and accompanying figures. Such modifications are intended to fall within the scope of the appended claims.

All references cited herein are incorporated herein by reference in their entirety and for all purposes to the same extent as if each individual publication or patent or patent application was specifically and individually indicated to be incorporated by reference in its entirety for all purposes.

1. A spray dried intermediate comprising, an amorphous form of a Compound having Formula (I): 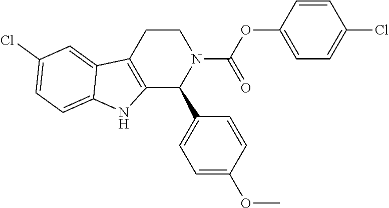 [SMILES: COc1ccc([C@H]2c3[nH]c4ccc(Cl)cc4c3CCN2C(=O)Oc2ccc(Cl)cc2)cc1]
and a polymer, wherein the polymer is a hydrophilic polymer.

2. The spray dried intermediate of claim 1, wherein the polymer is polyvinylpyrrolidone or hydroxypropyl lmethyl cellulose.

3. The spray dried intermediate of claim 2, wherein the polyvinylpyrrolidone is pylyvinylpyrrolidone K-30.

4. The spray dried intermediate of claim 2, wherein the hydroxypropyl methyl cellulose is hydroxypropyl methyl cellulose E5.

5. The spray dried intermediate of claim 1, wherein the Compound of Formula (I) is 40% by weight of the spray dried intermediate.

6. A method for preparing the spray dried intermediate of claim 1 comprising the steps of
co-dissolving the Compound of Formula (I) and the polymer in a solvent system comprising a solvent to form a liquid dispersion, then
removing the solvent by spray drying to provide the intermediate as a solid dispersion.

7. (canceled)

8. A pharmaceutical composition comprising the spray dried intermediate of claim 1 in intimate admixture with one or more pharmaceutically acceptable excipients to provide an oral dosage form.

9. The pharmaceutical composition of claim 8, wherein the Compound of Formula (I) is 20% by weight of the composition.

10. The pharmaceutical composition of claim 8, wherein the oral dosage form is a tablet.

11.-13. (canceled)

14. A method of treating a condition selected from the group consisting of a leukemia and an inflammatory disease in a subject in need thereof comprising, administering an effective amount of the pharmaceutical composition of claim 8 to the subject.

15. The method of claim 14, wherein the pharmaceutical composition is administered with food.

16. The method of claim 14, wherein the condition treated is leukemia selected from the group consisting of an acute or chronic leukemia.

17. The method of claim 14, wherein the condition treated is the inflammatory disease selected from the group consisting of rheumatoid arthritis and multiple sclerosis.

18. The pharmaceutical composition of claim 8, wherein the oral dosage form is a tablet or a capsule.

19. The method of claim 16, wherein the acute leukemia is selected from an acute lymphocytic leukemia; an acute myelocytic leukemia selected from a myeloblastic, promyelocytic, myelomonocytic, monocytic or erythroleukemia leukemia; or, myclodysplastic syndrome; and wherein the chronic leukemia is selected from a chronic myclocytic leukemia; a chronic granulocytic leukemia; a chronic lymphocytic leukemia; or, a hairy cell leukemia; or, polycythemia vera.

20. The method of claim 14, wherein the effective amount is administered to the subject in a weight based or fixed dose dosing regimen, wherein the dosing regimen maintains a target plasma concentration.
